(19) United States
(12) Patent Application Publication
(10) Pub. No.: US 2008/0194574 A1
(43) Pub. Date: Aug. 14, 2008

(54) Pyrazine Derivatives As Effective Compounds Against Infectious Diseases

(75) Inventors: Jan Eike Eikhoff, Munchen (DE); Mark Richard Ashton, Oxford (GB); Stephen Martin Courtney, Oxfordshire (GB); Christopher John Yarnold, Oxfordshire (GB); Maurizio Varrone, Oxfordshire (GB); Pui Leng Loke, Oxfordshire (GB); Thomas Herget, Darmstadt (DE); Wilfried Schwab, Neuried (DE); Doris Hafenbradl, Pullach (DE)

Correspondence Address: BROOKS KUSHMAN P.C., 1000 TOWN CENTER, TWENTY-SECOND FLOOR, SOUTHFIELD, MI, 48075, US

(73) Assignee: AXXIMA PHARMACEUTICALS AG, Munich (DE)

(21) Appl. No.: 10/596,508

(22) PCT Filed: Dec. 16, 2004
(86) PCT No.: PCT/EP2004/014371

§ 371 Date: Dec. 12, 2006

Related U.S. Application Data
(60) Provisional application No. 60/530,612, filed on Dec. 19, 2003.

(30) Foreign Application Priority Data
Dec. 16, 2003 (EP) 03029038.1

(51) Int. Cl. A61K 31/4985; C07D 487/04; C07D 241/10; A61K 31/4965; A61P 31/00; A61P 37/00; A61P 25/16; A61P 25/28

(52) U.S. Cl. 514/252.11; 544/350; 544/336; 514/252.1

(57) ABSTRACT

The present invention relates to pyrazine derivatives according to the general formula (I) and pharmaceutically acceptable salts thereof and their use as pharmaceutical compounds as well as the use of these compounds for the preparation of a pharmaceutical composition for the prophylaxis and/or treatment of infectious diseases, including opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke and especially for the treatment of herpesviral induced infections, including opportunistic infections as well as infections and diseases caused by human cytomegalovirus (HCMV).

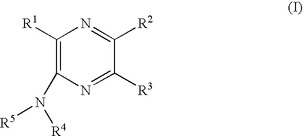

The present invention relates to pyrazine derivatives and pharmaceutically acceptable salts thereof and pharmaceutical compositions comprising at least one of these derivatives and/or pharmaceutically acceptable salts thereof, as well as the use of these derivatives especially for the prophylaxis and/or treatment of infectious diseases, including opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke.

### BACKGROUND OF THE INVENTION

The use of 2-amino-6-carba-disubstituted pyrazine compounds as protein kinase inhibitors, especially as inhibitors of JAK, is described in WO 02/060492. This patent application depicts the use of these compounds for example for the treatment of inflammatory or viral diseases such as Epstein Barr Virus, Hepatitis B, Hepatitits C or Varicella-Zoster Virus.

WO 02/24681 also describes the use of pyrazine derivatives as protein kinase inhibitors, especially of the vascular endothelial growth factor (VEGF) receptor tyrosine kinase. These pyrazine derivatives act as anti-tumor agents. Furthermore they are claimed for the treatment of angiogenesis, diabetic retinopathy, rheumatoid arthritis, endometriosis and psoriasis.

Furthermore, WO 02/40456 discloses piperazinylpyrazine compounds as agonists or antagonists of Serotonin 5HT-2 Receptor. These compounds are described for example for the treatment of obesity, epilepsy, sexual dysfunctions and urinary disorders.

It is object of the present invention to provide compounds and/or pharmaceutically acceptable salts thereof which can be used as pharmaceutically active agents, especially for prophylaxis and/or treatment of proliferative or infectious diseases, especially for the prophylaxis and/or treatment of herpes viral infections and/or associated diseases, including opportunistic infections, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke, methods to treat said diseases, as well as compositions comprising at least one of those compounds and/or pharmaceutically acceptable salts thereof as pharmaceutically active ingredients.

The object of the present invention is solved by the teaching of the independent claims. Further advantageous features, aspects and details of the invention are evident from the dependent claims, the description, and the examples of the present application.

The novel pyrazine derivatives according to the present invention are represented by the following general formula (I)

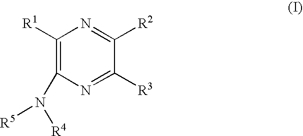

wherein
R¹ and R² are independently selected from the group comprising:
—H, —F, —Cl, —Br, —I, —NH₂, —OH, —SH, —CN, linear or branched, substituted or unsubstituted C₁-C₆ alkyl, linear or branched, substituted or unsubstituted C₂-C₆ alkenyl, linear or branched, substituted or unsubstituted C₂-C₆ alkinyl, substituted or unsubstituted C₃-C₈ cycloalkyl, linear or branched, substituted or unsubstituted C₁-C₆ alkoxy, linear or branched, substituted or unsubstituted C₁-C₆ haloalkyl or linear or branched, substituted or unsubstituted C₁-C₆ thioalkyl;
R³ is selected from substituted or unsubstituted C₃-C₈ cycloalkyl, substituted or unsubstituted aryl, substituted or unsubstituted heteroaryl, or substituted or unsubstituted heterocyclyl;
R⁴ is selected from —H or linear or branched C₁-C₆ alkyl;
R⁵ is selected from the group consisting of:
—H, substituted or unsubstituted, linear or branched C₁-C₆ alkyl, substituted or unsubstituted C₃-C₈ cycloalkyl, substituted or unsubstituted aryl, substituted or unsubstituted heteroaryl, substituted or unsubstituted heterocyclyl,
—(CH₂)ₘ—R⁶
   wherein m is selected to be an integer from 0 to 6 and
   R⁶ is selected from —H, substituted or unsubstituted C₃-C₈ cycloalkyl, substituted or unsubstituted aryl, substituted or unsubstituted heteroaryl, substituted or unsubstituted or heterocyclyl; and if m is selected to be an integer from 1 to 6, at least one, preferably one to two hydrogen atoms bonded to the —(CH₂)ₘ carbon chain are optionally substituted by —F, —Cl, —Br, —I, —OH, —NH₂, linear or branched C₁-C₆ alkyl or linear or branched C₁-C₆ alkoxy,
or —(CH₂)ₙ—N(R⁷)₂
   wherein n is an integer from 1 to 6 and R⁷ is selected from —H or linear or branched C₁-C₆ alkyl; if n is selected to be an integer from 1 to 6, at least one, preferably one to two hydrogen atoms bonded to the —(CH₂)ₙ carbon chain are optionally substituted by —F, —Cl, —Br, —I, —OH, —NH₂, linear or branched C₁-C₆ alkyl or linear or branched C₁-C₆ alkoxy
under the proviso, that if R₄ is —H, R₅ is different from —H;
or wherein
R⁴ and R⁵ together form a ring system represented by the formula (II)

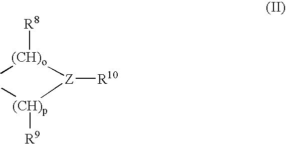

wherein o and p are independently selected to be an integer from 1 to 3,
Z is selected from CH or N,
each R⁸ and each R⁹ represent independently from each other —H, linear or branched C₁-C₆ alkyl or
—(CH₂)ᵤ—OH
   wherein u is selected to be an integer from 0 to 6 and if u is selected to be an integer from 1 to 6, at least one, preferably one to two hydrogen atoms bonded to the —(CH₂)ᵤ carbon chain are optionally substituted by —F, —Cl, —Br, —I, —OH, —NH₂, linear or branched C₁-C₆ alkyl or linear or branched C₁-C₆ alkoxy,
R¹⁰ is selected from the group comprising:
—H, linear or branched, substituted or unsubstituted C₁-C₆ alkyl, linear or branched, substituted or unsubstituted C₂-C₆ alkenyl, linear or branched, substituted or unsubstituted C₂-C₆ alkinyl, substituted or unsubstituted C₃-C₈ cycloalkyl, linear or branched, substituted or unsubstituted C₁-C₈ alkoxy, linear or branched, substituted or unsubstituted C₁-C₆ haloalkyl or linear or branched, substituted or unsubstituted C₁-C₆ thioalkyl, substituted or unsubstituted aryl, substituted or unsubstituted heteroaryl, or substituted or unsubstituted heterocyclyl,
—C(O)—R¹¹ or —(CH₂)_{q}—R¹¹
   wherein q is an integer from 0 to 6 and R₁₁ is selected from linear or branched, substituted or unsubstituted C₁-C₅ alkyl, linear or branched, substituted or unsubstituted C₂-C₆ alkenyl, linear or branched, substituted or unsubstituted C₁-C₆ alkinyl, substituted or unsubstituted C₃-C₈ cycloalkyl, linear or branched, substituted or unsubstituted C₁-C₆ alkoxy, linear or branched, substituted or unsubstituted C₁-C₆ haloalkyl or linear or branched, substituted or unsubstituted C₁-C₆ thioalkyl, substituted or unsubstituted aryl, substituted or unsubstituted heteroaryl, or substituted or unsubstituted heterocyclyl,
      and include stereoisomeric forms, prodrugs and pharmaceutically acceptable salts of these compounds.

As used herein, the term “C₁-C₈-alkyl” or “linear or branched C₁-C₆-alkyl” refers to —CH₃, —C₂H₅, —C₃H₇, —CH(CH₃)₂, —C₄H₉, —CH₂—CH(CH₃)₂, —CH(CH₃)—C₂H₅, —C(CH₃)₃, —CH(CH₃)C₃H₇, —CH₂—CH(CH₃)—C₂H₅, —CH(CH₃—CH(CH₃)₂, —C₅H₁₁, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)C₂H₅, —CH(CH₃)C₄H₉, —CH₂—CH(CH₃—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, and —CH(CH₃)—C(CH₃)₃.

Preferred are —CH₃, —C₂H₅, —C₃H₇, —CH(CH₃)₂, —C₄H₉, —CH₂—CH(CH₃)₂, —CH(CH₃)—C₂H₅, —C(CH₃)₃, and —C₅H₁. Especially preferred are —CH₃, —C₂H₅, —C₃H₇, and —CH(CH₃)₂.

As used herein, the term “C₂-C₆-alkenyl” or “linear or branched C₂-C₆-alkenyl” refers to —CH═CH₂, —CH₂—CH═CH₂, —CH═CH—CH₃, —C₂H₄—CH═CH₂, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —C₂H₄—C(CH₃)═CH₂, —CH₂—CH(CH₃)—CH═CH₂, —CH(CH₃)—CH₂—CH═CH₂, —CH₂—CH═C(CH₃)₂, —CH₂—C(CH₃)═CH—CH₃, —CH(CH₃—CH═CH—CH₃, —CH═CH—CH(CH₃)₂, —CH═C(CH₃)C₂H₅, —C(CH₃)═CH—C₂H₅, —C(CH₃)═C(CH₃)₂, —C(CH₃)₂—CH═CH₂, —CH(CH₃)C(CH₃)═CH₂, —C(CH₃)═CH—CH═CH₂, —CH═C(CH₃)—CH═CH₂, —CH═CH—C(CH₃)═CH₂, —C₄H₈CH═CH₂, —C₃H₆—CH═CH—CH₃, —C₂H₄—CH═CH—C₂H₅, —CH₂—CH═CH—C₃H₇, —CH═CH—C₄H₉, —C₃H₆—C(CH₃)═CH₂, —C₂H₄—CH(CH₃)CH═CH₂, —CH₂—CH(CH₃)—CH₂—CH═CH₂, —CH₂—CH═CH—CH₃, —CH(CH₃)C₂H₄—CH═CH₂, —C₂H₄—CH═C(CH₃)₂, —C₂H₄—C(CH₃)═CH—CH₃, —CH₂—CH(CH₃)—CH═CH—CH₃, —CH(CH₃)CH₂—CH═CH—CH₃, —CH₂—CH═CH—CH(CH₃)₂, —CH₂—CH═C(CH₃)C₂H₅, —CH₂—C(CH₃)═CH—C₂H₅, —CH(CH₃)—CH═CH—C₂H₅, —CH═CH—CH₂—CH(CH₃)₂, —CH═CH—CH(CH₃—C₂H₅, —CH═C(CH₃)C₃H₇, —C(CH₃)═CH—C₃H₇, —C(CH₃)═CH₂, —CH₂—CH(CH₃)C(CH₃)═CH₂, —CH(CH₃)CH₂—C(CH₃)═CH₂, —CH(CH₃)CH(CH₃)—CH═CH₂, —CH₂—C(CH₃)₂—CH═CH₂, —C(CH₃)₂—CH₂—CH═CH₂, —CH₂—C(CH₃)═C(CH₃)₂, —CH(CH₃)—CH═C(CH₃)₂, —C(CH₃)₂—CH═CH—CH₃, —CH(CH₃)—C(CH₃)═CH—CH₃, —CH═C(CH₃)—CH(CH₃)₂, —C(CH₃)═CH—CH(CH₃)₂, —C(CH₃)═C(CH₃)—C₂H₅, —C(CH₃)₂—C(CH₃)═CH₂, —CH(C₂H₅)—C(CH₃)═CH₂, —C(CH₃)(C₂H₅)—CH═CH₂, —CH(CH₃)—C(C₂H₅)═CH₂, —CH₂—C(C₂H₅)═CH—CH₃, —C[CH₂—CH(CH₃)₂]═CH₂, —C₂H₄—CH═CH—CH═CH₂, —CH₂—CH═CH—CH₂—CH═CH₂, —CH═CH—C₂H₄—CH═CH₂, —CH═CH—C(CH₃)₃, —CH₂—CH═CH—CH═CH—CH₃, —CH═CH—CH₂—CH═CH—CH₃, —C[CH(CH₃)(C₂H₅)]═CH₂, —CH═CH—CH═CH—C₂H₅, —CH₂—CH═CH—C(CH₃)═CH₂, —C(C₂H₅)═CH—C₂H₅, —CH₂—CH═C(CH₃)CH═CH₂, —CH₂—C(CH₃)═CH—CH═CH₂, —C(C₂H₅)═C(CH₃)₂, —CH(CH₃)—CH═CH—CH═CH₂, —CH═CH—CH₂—C(CH₃)═CH₂, —C(C₄H₉)═CH₂, —CH═CH—CH(CH₃)—CH═CH₂, —CH═C(CH₃)—CH₂—CH═CH₂, —C[C(CH₃)₃]═CH₂, —C(CH₃)═CH—CH₂—CH═CH₂, CH═CH—CH═C(CH₃)₂, —CH(C₂H₅)—CH═CH—CH₃, —CH═CH—C(CH₃)═CH—CH₃, —CH═C(CH₃)CH═CH—CH₃, —C(C₃H₇)═CH—CH₃, —C(CH₃)═CH—CH═CH—CH₃, —CH═C(CH₃)—C(CH₃)═CH₂, —CH₂—C(C₃H₇)═CH₂, —C(CH₃)═CH—C(CH₃)═CH₂, —C(CH₃)═C(CH₃)CH═CH₂, and —CH═CH—CH═CH—CH═CH₂.

Preferred are —CH═CH₂, —CH₂—CH═CH₂, —C(CH₃)═CH₂, —CH═CH—CH₃, —C₂H₄—CH═CH₂, —CH₂—CH═CH—CH₃. Especially preferred are —CH═CH₂, —CH₂—CH═CH₂, and —CH═CH—CH₃.

As used herein, the term “C₂-C₆-alkynyl” or “linear or branched C₂-C₆-alkynyl” refers to —C≡CH, —C≡C—CH₃, —CH₂—C≡CH, —C₂H₄—C≡CH, —CH₂—C≡C—CH₃, —C≡C—C₂H₅, —C₃H₆—C≡CH, —C₂H₄—C≡C—CH₃, —CH₂—C≡C—C₂H₅, —C≡C—C₃H₇, —CH(CH₃)—C≡CH, —CH₂—CH(CH₃)—C≡CH, —CH(CH₃)—CH₂—C≡CH, —CH(CH₃)—C≡C—CH₃, —C₄H₈—C≡CH, —C₃H₆—C≡C—CH₃, —C₂H₄—C≡C—C₂H₅, —CH₂—C≡C₃H₇, —C₂H₄—CH(CH₃)C≡CH, —CH₂—CH(CH₃)—CH₂—C≡CH, —CH(CH₃)—C₂H₄—C≡CH, —CH₂—CH(CH₃)—C≡C—CH₃, —CH(CH₃)—CH₂—C≡C—CH₃, —CH(CH₃)—C≡C—C₂H₅, —CH₂—C≡C—CH(CH₃)₂, —C═C—CH(CH₃)C₂H₅, —C≡C—CH₂—CH(CH₃)₂, —C≡C—C₄H₉, —C≡C—C(CH₃)₃, —CH(C₂H₅)—C≡C—CH₃, —C(CH₃)₂—C≡C—CH₃, —CH(C₂H₅)CH₂—C≡CH, —CH₂—CH(C₂H₅)—C≡CH, —C(CH₃)₂—CH₂—C≡CH, —CH₂—C(CH₃)₂—C≡CH, —CH(CH₃)CH(CH₃)C≡CH, —CH(C₃H₇)—C≡CH, —C(CH₃)(C₂H₅)—C≡CH, —C≡C—C≡CH, —CH₂—C≡C—C≡CH, —C≡C—C≡C—CH₃, —CH(C≡CH)₂, —C₂H₄—C≡C—C≡CH, —CH₂—C≡C—CH₂—C≡CH, —C≡C—C₂H₄—C≡CH, —CH₂—C≡C—C≡C—CH₃, —C≡C—CH₂—C≡C—CH₃, —C≡C—C≡C—C₂H₅, —C≡C—CH(CH₃)—C≡CH, —CH(CH₃)C≡C—C≡CH, —CH(C≡CH)—CH₂—C—CH, —C(C≡CH)₂—CH₃, —CH₂—CH(C≡CH)₂, —CH(C≡CH)—C≡C—CH₃.

Preferred are —C≡CH, —C≡C—CH₃.

The following groups are especially preferred:

—CH₃, —C₂H₅, —C₃H₇, —CH(CH₃)₂, —C₄H₉, —CH₂—CH(CH₃)₂, —CH(CH₃)C₂H₅, —C(CH₃)₃, —C₅H₁₁, —CH₂—C(CH₃)₃, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₂H₄—CH(CH₃)₂, —C₆H₁₃, —C₃H₆—CH(CH₃)₂, —C₂H₄—CH(CH₃)—C₂H₅, —CH(CH₃—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)C(CH₃)₃, —CH═CH₂, —C≡CH, —CH₂—CH═CH₂, —C(CH₃)═CH₂, —CH═CH—CH₃, —C≡C—CH₃, —CH₂—C—CH, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C_CH, —C₃H₆CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)═CH—CH═CH₂, —CH═C(CH₃)—CH═CH₂, —CH═CH—C(CH₃)═CH₂, —CH₂—CH═C(CH₃)₂, —C(CH₃)═C(CH₃)₂, —C₃H₆—C—CH, —C═C—C₃H₇, —C₂H₄—C═C—CH₃, —CH₂—C≡C—C₂H₅, —CH₂—C≡C—CH═CH₂, —CH₂—CH═CH—C≡CH, —CH₂—C═C—C≡CH, —C≡C—CH═CH—CH₃, —CH═CH—C≡C—CH₃, —C≡C—C≡C—CH₃, —C≡C—CH₂CH═CH₂, —CH═CH—CH₂—C≡CH, —C═C—CH₂—C≡CH, —C(CH₃)═CH—CH═CH₂, —CH═C(CH₃)CH═CH₂, —CH═CH—C(CH₃)═CH₂, —C(CH₃)═CH—C—CH, —CH═C(CH₃)—C≡CH, —C₄H₈—C≡CH, —C≡C—C(CH₃)═CH₂, —C₄H₈—CH═CH₂, —CH═CH—C₄H₉, —C₃H₆—C═C—CH₃, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C≡C—C₄H₉, —CH₂—C≡C—C₃H₇ or C₂H₄—C≡C—C₂H₅.

The term linear or branched C₁-C₄ alkyl is meant to include the respective subgroup out of the above groups.

The term linear or branched C₁-C₆ alkoxy represents a —O—(C₁-C₆ alkyl) group, wherein C₁-C₆ alkyl is meant to include the respective subgroup out of the above groups. The term linear or branched C₁-C₄ alkoxy is meant to include the respective subgroup out of the above groups.

The term linear or branched C₁-C₆ haloalkyl represents an C₁-C₆ alkyl group as defined above, wherein one to three hydrogen atoms bonded to the carbon chain are optionally substituted by a halogen atom such as —F, —Cl, —Br or —I.

The term linear or branched C₁-C₄ haloalkyl is meant to include the respective subgroup out the above groups.

The term linear or branched C₁-C₆ thioalkyl represents a —S—(C₁-C₆ alkyl) group, wherein C₁-C₆ alkyl is defined as above and the term linear or branched C₁-C₄ thioalkyl is meant to include the respective subgroup out the above groups.

As used herein, the term “C₁-C₈-cycloalkyl” refers to

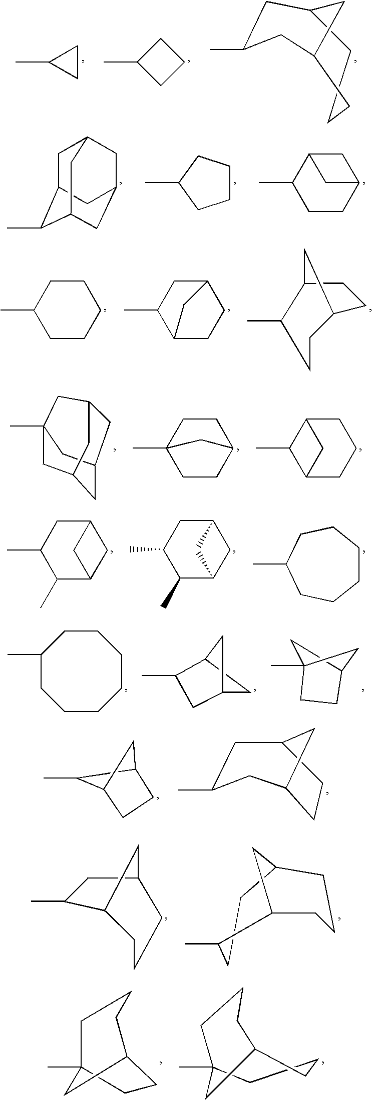

Preferred are the following cycloalkyls:

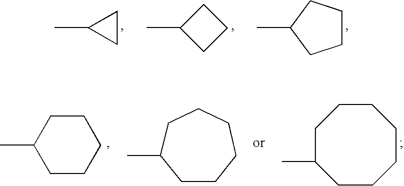

The functional groups and residues mentioned herein can be further substituted. Thus, the terms “substituted C₂-C₆ alkyl”, or “substituted C₂-C₆ alkenyl”, or “substituted C₂-C₆ alkinyl”, or “substituted C₃-C₈ cycloalkyl”, or “substituted C₁-C₆ alkoxy”, or “substituted C₁-C₆ haloalkyl”, or “substituted C₁-C₆ thioalkyl”, or “substituted aryl”, or “substituted heteroaryl”, or “substituted heterocyclyl” refers to “linear or branched C₂-C₆ alkyl”, or “linear or branched C₂-C₆ alkenyl”, or “linear or branched C₂-C₆ alkinyl”, or “C₃-C₈ cycloalkyl”, or “linear or branched C₁-C₆ alkoxy”, or “linear or branched C₁-C₆ haloalkyl” or “linear or branched C₁-C₆ thioalkyl”, or “aryl”, or “heteroaryl”, or “heterocyclyl” substituted with one, two, three, four, five or more, preferably with one or two substituents “Sub” independently selected from the following group:

—H, —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —NO₂, —F, —Cl, —Br, —I, —N₃, —CN, —OCN, —NCO, —SCN, —NCS, —CHO, —COCH₃, —COC₂H₅, —COC₃H₇, —CO-cyclo-C₃H₅, —COCH(CH₃)₂, —COC(CH₃)₃, —COOH, —COCN, —COOCH₃, —COOC₂H₅, —COOC₃H₇, —COO-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NH₂, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C(CH₃)₃, —SO₃H, —SO₃CH₃, —SO₃C₂H₅, —SO₃C₃H₇, —SO₃-cyclo-C₃H₅, —SO₃CH(CH₃)₂, —SO₃C(CH₃)₃, —OCF₃, —OC₂F₅, —O—COOCH₃, —O—COOC₂H₅, —O—COOC₃H₇, —O—COO-cyclo-C₃H₅, —O—COOCH(CH₃)₂, —O—COOC(CH₃)₃, —NH—CO—NH₂, —NH—CO—NHCH₃, —NH—CO—NHC₂H₅, —NH—CO—NHC₃H₇, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(cyclo-C₃H₅)₂, —NH—CO—N[CH(CH₃)₂]₂, —NH—CO—N[C(CH₃)₃]₂, —NH—CS—NH₂, —NH—CS—NHCH₃, —NH—CS—NHC₂H₅, —NH—CS—NHC₃H₇, —NH—CS—NH-cyclo-C₃H₅, —NH—CS—NH[CH(CH₃)₂], —NH—CS—NH[C(CH₃)₃], —NH—CS—N(CH₃)₂, —NH—CS—N(C₂H₅)₂, —NH—CS—N(C₃H₇)₂, —NH—CS—N(cyclo-C₃H₅)₂, —NH—CS—N[CH(CH₃)₂]₂, —NH—CS—N[C(CH₃)₃]₂, —NH—C(═NH)—NH₂, —NH—C(═NH)—NHCH₃, —NH—C(═NH)—NHC₂H₅, —NH—C(═NH)—NHC₃H₇, —NH—C(═NH)—NH-cyclo-C₃H₅, —NH—C(═NH)—NH[CH(CH₃)₂], —NH—C(═NH) NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NHC₃H₇, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(cyclo-C₃H₅)₂, —O—CO—N[CH(CH₃)₂]₂, —O—CO—N[C(CH₃)₃]₂, —O—CO—OCH₃, —O—CO—OC₂H₅, —O—CO—OC₃H₇, —O—CO—O-cyclo-C₃H₅, —O—CO—OCH(CH₃)₂, —O—CO—OC(CH₃)₃, —CH₂F —CHF₂, —CF₃, —CH₂Cl, —CHCl₂, —CCl₃, —CH₂Br —CHBr₂, —CBr₃, —CH₂I —CHI₂, —Cl₃, —CH₂—CH₂F —CH₂—CHF₂, —CH₂—CF₃, —CH₂—CH₂Cl, —CH₂—CHCl₂, —CH₂—CCl₃, —CH₂—CH₂Br CH₂—CHBr₂, —CH₂—CBr₃, —CH₂—CH₂₁—CH₂—CHI₂, —CH₂—C₁₃, —CH₃, —C₂H₅, —C₃H₇, -cyclo-C₃H₅, —CH(CH₃)₂, —C(CH₃)₃, —C₄H₉, —CH₂—CH(CH₃)₂, —CH(CH₃)—C₂H₅, —C(CH₃)₃, -Ph, —CH₂-Ph, —CPh₃, —CH═CH₂, —CH₂—CH═CH₂, —C(CH₃)═CH₂, —CH═CH—CH₃, —C₂H₄—CH═CH₂, —CH═C(CH₃)₂, —C≡CH, —C≡C—CH₃, —CH₂—C≡CH.

As used herein, the term “unsubstituted aryl” refers to phenyl, indenyl, indanyl, naphthyl, 1,2-dihydro-naphthyl, 2,3-dihydronaphthyl, 1,2,3,4-tetrahydronaphthyl (tetralinyl), fluorenyl, anthryl (anthracenyl), 9,10-dihydroanthryl, 1,2,3,4-tetrahydro-anthryl, 1,2,3,4,5,6,7,8-octahydro-anthryl, azulenyl, diphenylmethyl, benzyl, triphenylmethyl (trityl), styryl, naphthoquinonyl, acenaphthyl, anthraquinonyl, phenanthryl (phenanthrenyl).

As used herein, the term “unsubstituted heteroaryl” refers to heteroaromatic groups which have from 5 to 10 ring atoms, from 1 to 4 of which are selected from O, N and/or S. Preferred groups have 1 or 2 heteroatoms in a 5- or 6-membered aromatic ring. Mono and bicyclic ring systems are included. Typical heteroaryl groups include pyridyl, furyl, thienyl, pyrrolyl, oxazolyl, thiazolyl, imidazolyl, isoxazolyl, isothiazolyl, oxadiazolyl, pyridazinyl, pyrimidyl, pyrazinyl, 1,3,5-triazinyl, 1,2,3-triazolyl, 1,3,4-thiadiazolyl, indolizinyl, indolyl, isoindolyl, benzo[b]furyl, benzo[b]thienyl, indazolyl, benzimidazolyl, benzthiazolyl, purinyl, quinolizinyl, quinolyl, isoquinolyl, quinazolinyl, quinoxalinyl, 1,8-naphthyridinyl, tetrahydroquinolyl, benzooxazolyl, chrom-2-onyl, indazolyl, and the like.

As used herein, the term “unsubstituted C₁-C₆-heterocyclyl” or “unsubstituted C₁-C₆-heterocyclyl” refers to carbocycles having at least one heteroatom in the ring such as oxygen, nitrogen, or sulfur. Such heterocycles may be saturated or partially unsaturated but not aromatic. Examples for heterocyclic residues are 1,3-dioxolane, benzo[1,3]dioxolyl, pyrazolinyl, pyranyl, thiomorpholinyl, pyrazolidinyl, piperidyl, piperazinyl, 1,4-dioxanyl, imidazolinyl, pyrrolinyl, imidazolidinyl, morpholinyl, 1,4-dithianyl, pyrrolidinyl, oxozolinyl, oxazolidinyl, isoxazolinyl, isoxazolidinyl, thiazolinyl, thiazolidinyl, isothiazolinyl, isothiazolidinyl, dihydropyranl.

As used herein, the term “thioalkyl” refers to the residue —S—C₁-C₆-alkyl, wherein C₁-C₆-alkyl has the meanings as defined above. Preferably the following groups are concerned —S—CH₃, —S—C₂H₅, —S—C₃H₇, —S—CH(CH₃)₂, —S—C₄H₉, —S—CH₂—CH(CH₃)₂, —S—CH(CH₃)—C₂H₅, —S—C(CH₃)₃, and —S—C₅H₁₁. Most preferred are —S—CH₃, —S—C₂H₅, —S—C₃H₇, —S—CH(CH₃)₂, and —S—C(CH₃)₃.

As used herein, the term “alkyloxy” or “alkoxy” refers to the residue —O—C₁-C₆-alkyl, wherein C₁-C₆-alkyl has the meanings as defined above. Preferably the following groups are concerned —O—CH₃, —O—C₂H₅, —O—C₃H₇, —O—CH(CH₃)₂, —O—C₄H₉, —O—CH₂—CH(CH₃)₂, —O—CH(CH₃—C₂H₅, —O—C(CH₃)₃, and —O—C₅H₁₁. Most preferred are —O—CH₃, —O—C₂H₅, —O—C₃H₇, —O—CH(CH₃)₂, and —O—C(CH₃)₃.

As used herein, the term “acyl” or “C₁-C₆-alkanoyl” respectively refers to groups which are linked through a carbonyl (—C(═O)—) moiety and are represented by the general formula —CO—Ar or —CO—C₁-C₆-alkyl, wherein Ar refers to phenyl, substituted phenyl and heteroaryl and C₁-C₆-alkyl has the meanings as defined above. Preferred are —CO-Ph, —CO—CH₃, —CO—C₂H₅, —CO—C₃H₇, —CO—CH(CH₃)₂, —CO—C₄H₉, —CO—CH₂—CH(CH₃)₂, —CO—CH(CH₃)—C₂H₅, —CO—C(CH₃)₃, and —CO—C₅H₁₁. Most preferred are —CO—CH₃, —CO—C₂H₅, —CO—C₃H₇, —CO—CH(CH₃)₂, and —CO—C(CH₃)₃.

The term aryl denotes a mono- or bicyclic 6 to 10 membered ring system, preferably phenyl or napthyl.

The term “heteroaryl” is preferably meant to include a 5 to 10 membered mono- or bicyclic ringsystem, containing one to three heteroatoms independently selected from oxygen, sulfur or nitrogen and is preferably selected from the group consisting of:

thiophenyl, furanyl, pyrrolyl, imidazolyl, pyrazolyl, oxazolyl, isoxazolyl, thiazolyl, isothiazolyl, pyridinyl, pyrimidinyl, pyrazinyl, pyradizinyl, indolyl, benzo[b]thiophenyl, benzofuranyl, quinolinyl or isoquinolinyl.

The term heterocyclyl denotes a partially or fully saturated heteroaryl, consisting of a 5 to 10 membered mono or bicyclic ringsystem containing one to three heteroatoms independently selected from oxygen, sulfur or nitrogen and is preferably selected from the group comprising:

Pyrrolidinyl, tetrahydrothiophenyl, tetrahydrofuranyl, piperazinyl or morpholinyl.

The linear or branched C₁-C₆ alkyl, linear or branched C₂-C₆ alkenyl or linear or branched C₂-C₆ alkinyl, C₃-C₈ cycloalkyl, aryl, heteroaryl or heterocycyl can be either substituted or unsubstituted (i.e. non-substituted). Preferably, these groups, especially C₃-C₈ cycloalkyl, aryl, heteroaryl or heterocycyl, are optionally partially or fully substituted with members of the group comprising:

—F, —Cl, —Br, —I, —OH, —CN, —NO₂, linear or branched, substituted or unsubstituted C₁-C₆ alkyl, linear or branched, substituted or unsubstituted C₂-C₆ alkenyl, linear or branched, substituted or unsubstituted C₂-C₆ alkinyl, linear or branched, substituted or unsubstituted C₁-C₆ alkoxy, —O-phenyl, phenyl, linear or branched, substituted or unsubstituted C₁-C₆ haloalkyl, linear or branched, substituted or unsubstituted C₁-C₆ thioalkyl,
—(CH₂)ᵣ—X
wherein r is an integer from 0 to 6 and X is selected from

### —OH or —N(R¹²)₂

wherein each R₁₂ represents independently from each other —H or linear or branched C₁-C₆ alkyl,
   —-C(O)—R¹³ group
wherein R¹³ is selected from —H, X, or linear or branched C₁-C₆ alkyl,
   —NH—C(O)—R¹⁴ group
wherein R¹⁴ is selected from —H or linear or branched C₁-C₆ alkyl or
   —C(O)—NH—(CH₂)ₛ—X group,
wherein s is selected to be an integer from 0 to 6,
   or any other group as indicated in the claims of the present application.

In a preferred embodiment of the present invention R¹ in the compounds according to the general formula (I) is selected from —H, linear or branched, substituted or unsubstituted C₁-C₆ alkyl or —NH₂, preferably from —H, linear or branched, substituted or unsubstituted C₁-C₄ alkyl or —NH₂, more preferably from —H, —CH₃ or —NH₂ and most preferably from —H.

In a further preferred embodiment of the present invention R² in the compounds according to the general formula (I) is selected from —H, linear or branched, substituted or unsubstituted C₁-C₆ alkyl or —NH₂, preferably from —H, linear or branched, substituted or unsubstituted C₁-C₄ alkyl or —NH₂, more preferably from —H or —CH₃, and most preferably from —H.

In a further preferred embodiment of the present invention R³ in the compound according to the general formula (I) is selected from the group consisting of: substituted or unsubstituted aryl, substituted or unsubstituted heteroaryl, or substituted or unsubstituted heterocyclyl, and preferably is substituted or unsubstituted aryl, or substituted or unsubstituted heteroaryl.

In yet another preferred embodiment of the present invention R⁴ in the compound according the general formula (I) is selected from

—H or linear or branched C₁-C₄ alkyl, preferably —H or —CH₃, and is most preferably —H.
and R⁵ in the compound according to the general formula (I) is selected from linear or branched C₁-C₆ alkyl, preferably from linear or branched C₁-C₄ alkyl, more preferably from —C₂H₅ or —C₃H₇,
—(CH₂)ₘ—R⁶
wherein m is selected to be an integer from 0 to 4, preferably from 0 to 2, and wherein R⁶ is selected from the group comprising:
—H, substituted or unsubstituted aryl, preferably substituted or unsubstituted phenyl,
heteroaryl, selected from the group comprising:
pyridinyl, pyrrolyl, furanyl, thiophenyl, benzo[b]thiophenyl, benzofuranyl or indolyl, preferably 4-pyridinyl, 2-furanyl, 2-thiophenyl or 3-indolyl,
or
—(CH₂)ₙ—N(R⁷)₂,
   wherein n is selected to be an integer from 1 to 4, preferably from 1 to 2 and each R⁷ is independently selected from —H or linear or branched C₁-C₄ alkyl, and preferably is —H or —CH₃.
and R³ is selected from the group consisting of:
   substituted or unsubstituted aryl or substituted or unsubstituted heteroaryl, preferably aryl or bicyclic heteroaryl, more preferably phenyl, naphthyl, benzofuranyl or indolyl, and is most preferably phenyl.

In yet another preferred embodiment of the present invention R³ and R⁶ in the compound according to the general formula (I) are selected to be phenyl, each independently of the other partially or fully substituted with members selected from the group consisting of:

—F, —Cl, —Br, —I, —OH, linear or branched C₁-C₄ alkoxy, linear or branched C₁-C₄ alkyl, linear or branched C₁-C₄ haloalkyl, —CN,
—C(O)—NH—(CH₂)ₛ—N(R¹²)₂ group, wherein s is an integer from 0 to 4, preferably an integer from 0 to 3, and is most preferably 2 and wherein each R¹² represents independently from each other —H or linear or branched C₁-C₄ alkyl, and preferably is —H or —CH₃ or
—C(O)—R¹³ group
wherein R¹³ is selected to be —N(R¹²)₂
   wherein each R¹² represents independently from each other —H or linear or branched C₁-C₄ alkyl, and preferably is —H.

In yet another preferred embodiment of the present invention R³ and R⁶ in the compound according to the general formula (I) are preferably substituted with members selected from the group comprising:

—F, —Cl, —CN, —OH, —OCH₃, —CF₃, —CH₃ or —C(O)NH₂, and wherein each phenyl is preferably mono-, di- or trisubstituted, more preferably mono- or disubstituted.

In yet another preferred embodiment of the present invention R⁵ in the compound according to the general formula (I) is selected to be —(CH₂)ₙ—N(R⁷)₂, wherein n is selected to be an integer from 1 to 4, preferably from 1 to 3, and is most preferably 2, and wherein each R⁷ is independently selected from —H, linear or branched C₁-C₄ alkyl, preferably from —CH₃ and R³ in the compound according to the general formula (I) is selected from aryl or heteroaryl, preferably from aryl, and is most preferably naphthyl.

In another preferred embodiment of the present invention R⁴ and R⁵ in the compound according to the general formula (I) form a ring system represented by the formula (II)

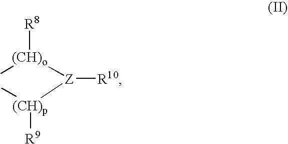

wherein o and p are independently selected to be 1 or 2, preferably o is selected to be 1 or 2 and p is selected to be 2, and most preferably o and p are both selected to be 2,
each R⁸ and each R⁹ is independently selected from the group comprising:
—(CH₂)ᵤ—OH, wherein u is an integer from 0 to 4, preferably from 0 to 2,
—H or linear or branched C₁-C₄ alkyl, preferably —H or —CH₃,
Z is N or CH,
R¹⁰ is selected from the consisting of:
—H, substituted or unsubstituted aryl, substituted or unsubstituted heteroaryl, substituted or unsubstituted heterocyclyl,
—(CH₂)_{q}—R¹¹ or —C(O)—R¹¹
   wherein q is selected to be an integer from 0 to 6,
   R¹¹ is selected to be a heteroaryl or heterocyclyl, preferably a 5 membered monocyclic heteroaryl or a 5 membered monocyclic heterocyclyl, and is most preferably thiophenyl, furanyl, pyrroyl or pyrrolidinyl, pyrrolidinyl.

In yet another preferred embodiment of the present invention, Z in the ring system according to formula (II) is selected to be CH.

In yet another preferred embodiment of the present invention o in the ring system according to the formula (II) is selected to be 1 and p is selected to be 2, R⁸ is —H, each R⁹ represents independently from each other —H or —CH₂—OH, R³ is a heteroaryl, preferably a bicyclic heteroaryl, and is most preferably benzofuranyl, and R¹⁰ is —H.

In yet another preferred embodiment of the present invention o and p in the ring system according to the general formula (II) are selected to be 2, each R⁸ and each R⁹ are selected to be —H, R³ is selected to be substituted or unsubstituted aryl or substituted or unsubstituted heteroaryl, and R¹⁰ is selected to be substituted or unsubstituted aryl, preferably substituted or unsubstituted phenyl.

In yet another preferred embodiment of the present invention R³ in the compound according to the general formula (I) is selected from the group comprising: Furanyl, thiophenyl, pyrrolidinyl, preferably 3-furanyl or 3-thiophenyl, benzo[b]-thiophenyl, benzofuranyl, indolyl, preferably 3-benzo[b]thiophenyl, naphthyl or phenyl, each of these moieties being optionally fully or partially substituted, preferably mono- or disubstituted, with members of the group consisting of:
—F, —Cl, —Br, —I, preferably —F or —Cl,
linear or branched C₁-C₄ alkoxy, preferably —OCH₃,
linear or branched C₁-C₄ thioalkyl, preferably —SCH₃,
—(CH₂)ᵣ—N(R¹²)₂,
   wherein r is an integer from 0 to 4, preferably from 0 to 2, and is most preferably 0 and wherein each R¹² represents independently from each other —H or linear or branched C₁-C₄ alkyl, preferably —CH₃,
or
—C(O)—R¹³
wherein R¹³ is selected to be linear or branched C₁-C₄ alkyl, preferably —CH₃,
   wherein, within this embodiment it is especially preferred that the group R³ in the compound according to general formula (I) is phenyl, optionally partially or fully, preferably mono- or disubstituted, with members of the above listed group.

In a further preferred embodiment of the present invention Z in the ring system according formula (II) in the compound according to the general formula (I) is selected to be N and o and p are independently selected to be an integer from 1 to 3, preferably o and p are selected to be 2, each R⁸ and each R⁹ are independently selected from —H or —CH₃, R³ is selected from aryl or heteroaryl, and R¹⁰ is selected from the group comprising:

aryl, heteroaryl or —C(O)—R¹¹ or —(CH₂)_{q}—R¹¹, wherein R¹¹ is selected from heteroaryl or heterocyclyl and q is an integer from 0 to 4, preferably from 0 to 2.

In yet another preferred embodiment of the present invention each R⁸ and one R⁹ in the ring system according to formula (II) in the compound according to general formula (I) represent —H, and one R⁹ represents —CH₃.

In yet another preferred embodiment of the present invention R¹⁰ in the ring system according to formula (II) in the compound according to general formula (I) is phenyl, optionally fully or partially substituted, preferably monosubstituted, with linear or branched C₁-C₄ alkyl, preferably —CH₃,

and wherein R³ is selected from the group comprising:
substituted or unsubstituted naphthyl, bicyclic heteroaryl, preferably benzo[b]thiophenyl or phenyl, optionally fully or partially substituted, preferably mono- or disubstituted, with members of the group consisting of:
linear or branched C₁-C₄ alkyl, preferably —CH₃, linear or branched C₁-C₄ thioalkyl, preferably —S—CH₃ or —CO(NR¹²)₂, wherein each R¹² represents independently from each other —H or linear or branched C₁-C₄ alkyl, preferably —H, and wherein within this embodiment it is especially preferred that R³ is phenyl, optionally substituted in the above outlined manner.

In another preferred embodiment of the present invention each R⁸ and R⁹ in the ring system according to formula (II) in the compound according to general formula (I) represents —H.

In yet another preferred embodiment of the present invention R¹⁰ in the ring system according to formula (II) in the compound according to general formula (I) is selected from the group consisting of:

phenyl, pyridinyl, pyrimidinyl, pyrazinyl, benzo[b]thiophenyl, benzofuranyl, indolyl, —C(O)—R¹¹, wherein R¹¹ is a monocyclic heteroaryl, preferably furanyl, or (CH₂)_{q}—R¹¹, wherein q is selected to be 2 and R¹¹ is a monocyclic heterocyclyl, preferably pyrrolidinyl.

In yet another preferred embodiment of the present invention R¹⁰ in the ring system according to formula (II) in the compound according to the general formula (I) is selected from the group comprising:

Pyridinyl, pyrazinyl, indolyl or phenyl.

In yet another preferred embodiment of the present invention R¹⁰ in the ring system according to formula (II) in the compound according to the general formula (I) is pyridinyl, preferably 2-pyridinyl or

4-pyridinyl, more preferably 4-pyridinyl or phenyl, which is optionally fully or partially substituted, preferably mono- or disubstituted, with members of the group consisting of:
—F, —Cl, —Br, or —I, preferably —F or —Cl,
linear or branched C₁-C₄ alkoxy, preferably —OCH₃,
linear or branched C₁-C₄ alkyl, preferably —CH₃,
linear or branched C₁-C₄ haloalkyl, preferably —CF₃
—CN or —(CO)—R¹³, wherein R¹³ represents linear or branched C₁-C₄ alkyl, preferably —CH₃.

In yet another preferred embodiment of the present invention R³ in the compound according to the general formula (I) is selected from the group consisting of:

phenyl, naphthyl,
pyrrolyl, thiophenyl, furanyl, preferably 2-thiophenyl, 3-thiophenyl, 2-furanyl or 3-furanyl,
pyridinyl, preferably 3-pyridinyl,
benzo[b]thiophenyl, benzofuranyl or indolyl, preferably benzo[b]thiophenyl or benzofuranyl and wherein all of these group members may be substituted or unsubstituted.

In yet another preferred embodiment of the present invention R³ in the compound according to the general formula (I) is naphthyl, optionally partially or fully substituted with C₁-C₄ alkoxy, preferably with —OCH₃, thiophenyl, optionally partially or fully substituted with —(CO)—R¹³, wherein R¹³ represents linear or branched C₁-C₄ alkyl, preferably —CH₃ and naphthyl or thiophenyl are preferably monosubstituted.

In yet another preferred embodiment of the present invention R³ in the compound according to the general formula (I) is phenyl, optionally partially or fully substituted with members of the group comprising:

—F, —Cl, —Br, —I, —CN, linear or branched C₁-C₄ alkoxy, —OPh, linear or branched C₁-C₄ alkyl, phenyl, linear or branched C₁-C₄ haloalkyl, linear or branched C₁-C₄ thioalkyl,
—(CH₂)ᵣ—X,
wherein X is selected to be —OH or —(NR¹²)₂
   wherein r is an integer from 0 to 2, preferably r is 0 or 1 and each R¹² represents independently from each other —H or linear or branched C₁-C₄ alkyl, preferably —H or —CH₃,

### —(CO)—R¹³

wherein R¹³ represents linear or branched C₁-C₄ alkyl, preferably —CH₃ or —(NR¹²)₂, wherein each R¹² represents independently from each other —H or linear or branched C₁-C₄ alkyl, preferably —H,

### —NH—(CO)—R¹⁴

wherein R¹⁴ is selected from —H or linear or branched C₁-C₄ alkyl, preferably —CH₃,
   or —C(O)—NH—(CH₂)ₛ—OH, wherein s is selected to be an integer from 0 to 4, and is preferably 2.

In yet another preferred embodiment of the present invention R³ in the compound according to the general formula (I) is phenyl, substituted with members selected from the group comprising:

—F, —Cl, —Br, preferably —F or —Cl,
—O—CH₃, —O—C₂H₅, —SCH₃, —CH₃, —CH(CH₃)₂, —C(CH₃)₃, —CH₂OH, —N(CH₃)₂, —CF₃ or —C(O)NH₂ and wherein the phenyl is preferably mono-, di- or trisubstituted, more preferably mono- or disubstituted.

In a further preferred embodiment of the present invention R¹ and R² in the compound according to the general formula (I) are —H.

The following subformula (IIIA)-(IIIG) of formula (I) are especially preferred:

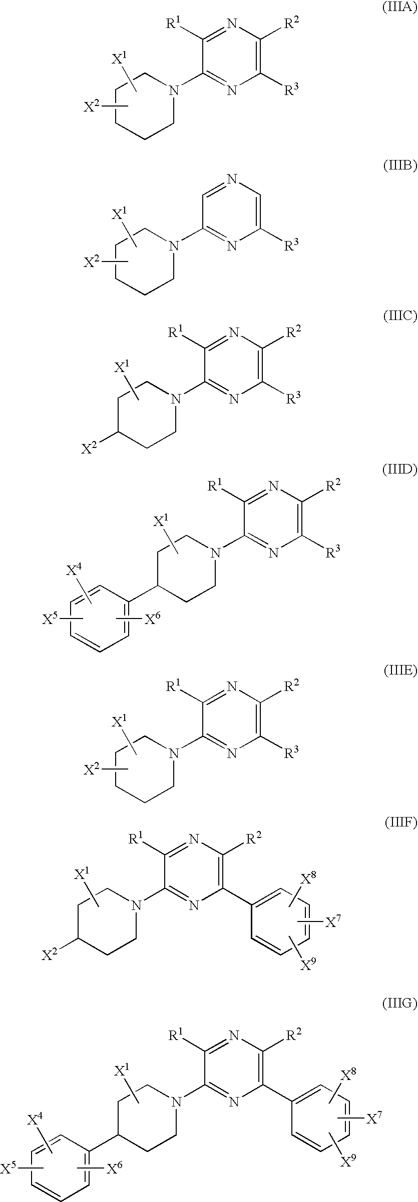

wherein
R¹, R², and R³ have the meaning as defined above and
X¹, and X² are independently of each other linear or branched, substituted or unsubstituted C₂-C₆ alkyl, linear or branched, substituted or unsubstituted C₂-C₆ alkenyl, linear or branched, substituted or unsubstituted C₂-C₆ alkinyl, substituted or unsubstituted C₃-C₈ cycloalkyl, linear or branched, substituted or unsubstituted C₁-C₆ alkoxy, linear or branched, substituted or unsubstituted C₁-C₆ haloalkyl, linear or branched, substituted or unsubstituted C₁-C₆ thioalkyl, substituted or unsubstituted aryl, substituted or unsubstituted heteroaryl, substituted or unsubstituted heterocyclyl and preferably substituted aryl, substituted heteroaryl, substituted heterocyclyl, more preferably substituted aryl, and most preferably substituted phenyl. Preferably the substituted phenyl bears one or two substitutents and also preferably in para position. The substitutents can independently of each be selected from the group defined as “Sub” as outlined above; and
X⁴, X⁵, X⁶, X⁷, X⁸, and X⁹ represent independently of each other a substituent defined as “Sub” as described above. Further especially preferred are compounds wherein R³ and X² are independently of each other selected from substituted or unsubstituted aryl, substituted or unsubstituted heteroaryl, substituted or unsubstituted heterocyclyl and preferably from substituted or unsubstituted aryl and substituted or unsubstituted heteroaryl.

Further especially preferred subformula of formula (I) are (IVA)-(IVG):

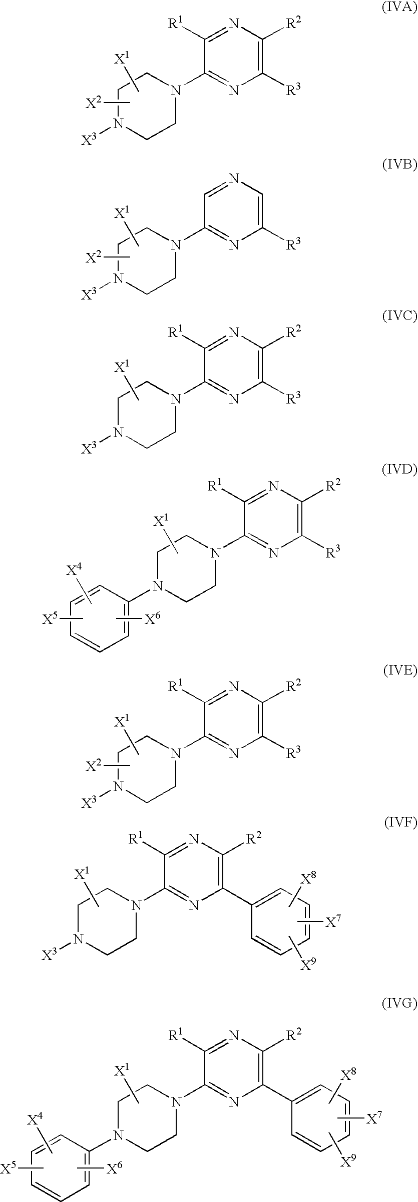

wherein
R¹, R², and R³ have the meaning as defined above and
X¹, X², and X³ are independently of each other linear or branched, substituted or unsubstituted C₂-C₆ alkyl, linear or branched, substituted or unsubstituted C₂-C₆ alkenyl, linear or branched, substituted or unsubstituted C₂-C₆ alkinyl, substituted or unsubstituted C₃-C₈ cycloalkyl, linear or branched, substituted or unsubstituted C₁-C₆ alkoxy, linear or branched, substituted or unsubstituted C₁-C₆ haloalkyl, linear or branched, substituted or unsubstituted C₁-C₆ thioalkyl, substituted or unsubstituted aryl, substituted or unsubstituted heteroaryl, substituted or unsubstituted heterocyclyl and preferably substituted aryl, substituted heteroaryl, substituted heterocyclyl, more preferably substituted aryl, and most preferably substituted phenyl. Preferably the substituted phenyl bears one or two substitutents and also preferably in para position. The substitutents can independently of each be selected from the group defined as “Sub” as outlined above; and
X⁴, X⁵, X⁶, X⁷, X⁸, and X⁹ represent independently of each other a substituent defined as “Sub” as described above. Further especially preferred are compounds wherein R³ and X³ are independently of each other selected from substituted or unsubstituted aryl, substituted or unsubstituted heteroaryl, substituted or unsubstituted heterocyclyl and preferably from substituted or unsubstituted aryl and substituted or unsubstituted heteroaryl.

Further preferred subformula of formula (I) can be described by the general formula (IIIH), (IVH), (VA), (VB), (VC), and (VI):

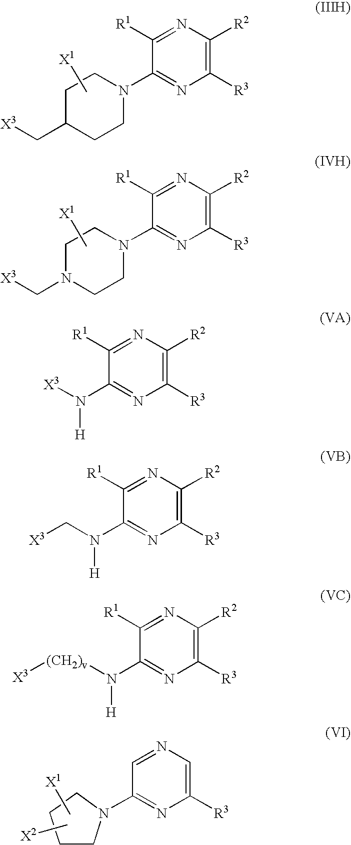

wherein
R¹, R², and R³ have the meaning as defined above and v is an integer between 2 and 10,
X¹, X², and X³ are independently of each other linear or branched, substituted or unsubstituted C₂-C₆ alkyl, linear or branched, substituted or unsubstituted C₂-C₆ alkenyl, linear or branched, substituted or unsubstituted C₂-C₆ alkinyl, substituted or unsubstituted C₃-C₈ cycloalkyl, linear or branched, substituted or unsubstituted C₁-C₆ alkoxy, linear or branched, substituted or unsubstituted C₁-C₆ haloalkyl, linear or branched, substituted or unsubstituted C₁-C₆ thioalkyl, substituted or unsubstituted aryl, substituted or unsubstituted heteroaryl, substituted or unsubstituted heterocyclyl and preferably substituted aryl, substituted heteroaryl, substituted heterocyclyl, more preferably substituted aryl, and most preferably substituted phenyl. Preferably the substituted phenyl bears one or two substitutents and also preferably in para position. The substitutents can independently of each be selected from the group defined as “Sub” as outlined above.

Preferably R³ and X³ are independently of each other selected from substituted or unsubstituted aryl, substituted or unsubstituted heteroaryl, substituted or unsubstituted heterocyclyl and more preferably from substituted or unsubstituted aryl and substituted or unsubstituted heteroaryl. Preferably X¹ and X² represent independently of each other a substituent defined as “Sub” as described above. X² is preferred in position 2 of the pyrrolidine moiety. R¹ and R² are preferably hydrogen.

In yet another preferred embodiment of the present invention the compound according to the general formula (I) is selected from the group of compounds depicted in Table 1.

Additionally, this table shows that the inventive compounds of the general formula (I) are potent inhibitors of UL 97 kinase activity. Furthermore, it was shown, that compounds according to the general formula (I) inhibit efficiently human Cytomegalovirus replication by inhibition of UL 97 in intact cells (FIG. 2).

The compounds listed in Table 1 belonging to the activity class A, do have an half maximal inhibition value (IC₅₀) of 10-100 μM and an inhibition value of more than 15% at 10 μM or an inhibition value of more than 50% at 100 μM was obtained.

The compounds belonging to the activity class B show an IC₅₀ of 1-10 μM and inhibition value of more than 50% at 10 μM. With compounds belonging to the activity class C an IC₅₀ of less than 1 μM was obtained.

TABLE I
Claimed compounds according to the present invention

| | | | LC ret. | | Activity |
|---|---|---|---|---|---|
| Comp. | | | time | MS | Class |
| No. | MW | IUPAC name | [min] | [M + H]+ | [A, B, C] |
| 1 | 426.52 | 4-(3,4-Dimethoxy-phenyl)-6′-naphthalen-2-yl- | 9.77* | 427.2 | C |
| | | 3,4,5,6-tetrahydro-2H-[1,2′]bipyrazinyl | | | |
| 2 | 351.84 | 6′-(3-Chloro-phenyl)-4-pyridin-4-yl-3,4,5,6- | | | C |
| | | tetrahydro-2H-[1,2′]bipyrazinyl | | | |
| 3 | 359.48 | 6′-(4-Isopropyl-phenyl)-4-pyridin-4-yl-3,4,5,6- | | | C |
| | | tetrahydro-2H-[1,2′]bipyrazinyl | | | |
| 4 | 333.40 | 3-(4-Pyridin-4-yl-3,4,5,6-tetrahydro-2H- | 8.45* | 334.2 | B |
| | | [1,2′]bipyrazinyl-6′-yl)-phenol | | | |
| 5 | 418.54 | 4-(3,4-Dimethoxy-phenyl)-6′-(4-isopropyl- | 9.93* | 419.2 | B |
| | | phenyl)-3,4,5,6-tetrahydro-2H-[1,2′]-bipyrazinyl | | | |
| 6 | 406.49 | {4-[4-(3,4-Dimethoxy-phenyl)-3,4,5,6-tetrahydro- | 1.12 | 407 | B |
| | | 2H-[1,2′]bipyrazinyl-6′-yl]-phenyl}-methanol | | | |
| 7 | 355.37 | [6-(3-Fluoro-phenyl)-pyrazin-2-yl]-(3,4,5- | 9.00* | 356.1 | B |
| | | trimethoxy-phenyl)-amine | | | |
| 8 | 335.39 | 6′-(3-Fluoro-phenyl)-4-pyridin-4-yl-3,4,5,6- | 9.43* | 336.1 | A |
| | | tetrahydro-2H-[1,2′]bipyrazinyl | | | |
| 9 | 326.40 | (6-Naphthalen-2-yl-pyrazin-2-yl)-(2-pyridin-4-yl- | 8.87* | 327.2 | C |
| | | ethyl)-amine | | | |
| 10 | 367.46 | 6′-Naphthalen-2-yl-4-pyridin-2-yl-3,4,5,6- | 9.82* | 368.2 | C |
| | | tetrahydro-2H-[1,2′]bipyrazinyl | | | |
| 11 | 315.74 | 4-[6-(3-Chloro-4-fluoro-phenylamino)-pyrazin-2- | 9.52* | 316.1 | C |
| | | yl]-phenol | | | |
| 12 | 347.42 | 6′-(4-Methoxy-phenyl)-4-pyridin-4-yl-3,4,5,6- | 9.27* | 348.2 | C |
| | | tetrahydro-2H-[1,2′]bipyrazinyl | | | |
| 13 | 306.41 | N,N,N′-Trimethyl-N′-(6-naphthalen-2-yl-pyrazin- | | | C |
| | | 2-yl)-ethane-1,2-diamine | | | |
| 14 | 351.84 | 6′-(4-Chloro-phenyl)-4-pyridin-2-yl-3,4,5,6- | 9.60* | 352.1 | A |
| | | tetrahydro-2H-[1,2′]bipyrazinyl | | | |
| 15 | 360.42 | 4-(4-Pyridin-2-yl-3,4,5,6-tetrahydro-2H- | 0.94 | 361 | C |
| | | [1,2′]bipyrazinyl-6′-yl)-benzamide | | | |
| 16 | 360.47 | Dimethyl-[3-(4-pyridin-4-yl-3,4,5,6-tetrahydro-2H- | | | A |
| | | [1,2′]bipyrazinyl-6′-yl)-phenyl]-amine | | | |
| 17 | 387.53 | 6′-Naphthalen-2-yl-4-(2-pyrrolidin-1-yl-ethyl)- | | | C |
| | | 3,4,5,6-tetrahydro-2H-[1,2′]bipyrazinyl | | | |
| 18 | 306.37 | [6-(4-Methoxy-phenyl)-pyrazin-2-yl]-(2-pyridin-3- | 7.9* | 307.2 | C |
| | | yl-ethyl)-amine | | | |
| 19 | 353.38 | 6′-(2,4-Difluoro-phenyl)-4-pyridin-2-yl-3,4,5,6- | 9.27* | 354.2 | A |
| | | tetrahydro-2H-[1,2′]bipyrazinyl | | | |
| 20 | 419.49 | 4-[4-(3,4-Dimethoxy-phenyl)-3,4,5,6- | 0.94 | 420 | C |
| | | tetrahydro-2H-[1,2′]bipyrazinyl-6′- | | | |
| | | yl]-benzamide | | | |
| 21 | 360.42 | 4-(4-Pyridin-4-yl-3,4,5,6-tetrahydro-2H- | 0.79 | 361 | C |
| | | [1,2′]bipyrazinyl-6′-yl)-benzamide | | | |
| 22 | 420.91 | 3-[6-(3-Chloro-4-cyano-phenylamino)-pyrazin-2- | | | C |
| | | yl]-N-(2-dimethylamino-ethyl)-benzamide | | | |
| 23 | 291.31 | (6-Benzofuran-2-yl-pyrazin-2-yl)-furan-2- | | | A |
| | | ylmethyl-amine | | | |
| 24 | 295.34 | [1-(6-Benzofuran-2-yl-pyrazin-2-yl)-pyrrolidin-2- | | | B |
| | | yl]-methanol | | | |
| 25 | 256.31 | 4-(6-Propylamino-pyrazin-2-yl)-benzamide | | | C |
| 26 | 285.74 | [6-(4-Chloro-phenyl)-pyrazin-2-yl]-furan-2- | 9.2* | 286.1 | C |
| | | ylmethyl-amine | | | |
| 27 | 295.77 | Benzyl-[6-(4-chloro-phenyl)-pyrazin-2-yl]-amine | 9.63* | 296.1 | A |
| 28 | 304.35 | 4-(6-Benzylamino-pyrazin-2-yl)-benzamide | 7.4* | 305.1 | C |
| 29 | 331.30 | 4-[6-(3-Trifluoromethyl-phenylamino)-pyrazin-2- | 9.47* | 332.1 | C |
| | | yl]-phenol | | | |
| 30 | 283.35 | 4-{6-[(Thiophen-2-ylmethyl)-amino]-pyrazin-2-yl}- | 8.25* | 284.1 | C |
| | | phenol | | | |
| 31 | 323.42 | 4-Pyridin-2-yl-6′-thiophen-2-yl-3,4,5,6-tetrahydro- | 8.97* | 324.1 | A |
| | | 2H-[1,2′]bipyrazinyl | | | |
| 32 | 385.40 | 4-Pyridin-4-yl-6′-(3-trifluoromethyl-phenyl)- | 9.93* | 386.1 | C |
| | | 3,4,5,6-tetrahydro-2H-[1,2′]bipyrazinyl | | | |
| 33 | 369.83 | 6′-(3-Chloro-4-fluoro-phenyl)-4-pyridin-4-yl- | 10.3* | 370.1 | C |
| | | 3,4,5,6-tetrahydro-2H-[1,2′]bipyrazinyl | | | |
| 34 | 377.41 | 4-[4-(Furan-2-carbonyl)-3,4,5,6-tetrahydro-2H- | 6.62* | 378.1 | C |
| | | [1,2′]bipyrazinyl-6′-yl]-benzamide | | | |
| 35 | 357.42 | 4-{6-[2-(1H-Indol-3-yl)-ethylamino]-pyrazin-2-yl}- | 1.14 | 358 | C |
| | | benzamide | | | |
| 36 | 412.50 | N-{3-[4-(1H-Indol-4-yl)-3,4,5,6-tetrahydro-2H- | | | A |
| | | [1,2′]bipyrazinyl-6′-yl]-phenyl}-acetamide | | | |
| 37 | 445.53 | 4-(1H-Indol-4-yl)-6′-(3,4,5-trimethoxy-phenyl)- | 9.05* | 446.2 | A |
| | | 3,4,5,6-tetrahydro-2H-[1,2′]bipyrazinyl | | | |
| 38 | 385.47 | 4-(1H-Indol-4-yl)-6′-(4-methoxy-phenyl)-3,4,5,6- | 9.42* | 386.2 | B |
| | | tetrahydro-2H-[1,2′]bipyrazinyl | | | |
| 39 | 404.48 | N-(2-Hydroxy-ethyl)-3-(4-pyridin-4-yl-3,4,5,6- | 7.47* | 405.2 | A |
| | | tetrahydro-2H-[1,2′]bipyrazinyl-6′-yl)-benzamide | | | |
| 40 | 242.28 | 4-(6-Ethylamino-pyrazin-2-yl)-benzamide | 6.53* | 243.1 | B |
| 41 | 412.44 | 6′-(2,4-Difluoro-phenyl)-4-(3,4-dimethoxy- | 9.23* | 413.1 | A |
| | | phenyl)-3,4,5,6-tetrahydro-2H-[1,2′]bipyrazinyl | | | |
| 42 | 375.86 | 2-[6′-(4-Chloro-phenyl)-2,3,5,6-tetrahydro- | | | B |
| | | [1,2′]bipyrazinyl-4-yl]-benzonitrile | | | |
| 43 | 426.52 | 4-(3,4-Dimethoxy-phenyl)-6′-naphthalen-1-yl- | | | A |
| | | 3,4,5,6-tetrahydro-2H-[1,2′]bipyrazinyl | | | |
| 44 | 382.49 | 4-(3,4-Dimethoxy-phenyl)-6′-thiophen-2-yl- | | | B |
| | | 3,4,5,6-tetrahydro-2H-[1,2′]bipyrazinyl | | | |
| 45 | 420.52 | 4-(3,4-Dimethoxy-phenyl)-6′-(4-ethoxy-phenyl)- | 1.37 | 421 | B |
| | | 3,4,5,6-tetrahydro-2H-[1,2′]bipyrazinyl | | | |
| 46 | 353.38 | 6′-(2,4-Difluoro-phenyl)-4-pyridin-4-yl-3,4,5,6- | 9.52* | 354.2 | B |
| | | tetrahydro-2H-[1,2′]bipyrazinyl | | | |
| 47 | 351.84 | 6′-(4-Chloro-phenyl)-4-pyridin-4-yl-3,4,5,6- | | | B |
| | | tetrahydro-2H-[1,2′]bipyrazinyl | | | |
| 48 | 287.27 | [6-(2,4-Difluoro-phenyl)-pyrazin-2-yl]-furan-2- | | | B |
| | | ylmethyl-amine | | | |
| 49 | 317.40 | 6′-Phenyl-4-pyridin-2-yl-3,4,5,6-tetrahydro-2H- | | | B |
| | | [1,2′]bipyrazinyl | | | |
| 50 | 345.45 | 6′-(3,4-Dimethyl-phenyl)-4-pyridin-2-yl-3,4,5,6- | | | B |
| | | tetrahydro-2H-[1,2′]bipyrazinyl | | | |
| 51 | 279.34 | [6-(3,4-Dimethyl-phenyl)-pyrazin-2-yl]-furan-2- | | | A |
| | | ylmethyl-amine | | | |
| 52 | 347.42 | [3-(4-Pyridin-4-yl-3,4,5,6-tetrahydro-2H- | 8.07* | 348.2 | A |
| | | [1,2′]bipyrazinyl-6′-yl)-phenyl]-methanol | | | |
| 53 | 351.84 | 6′-(3-Chloro-phenyl)-4-pyridin-2-yl-3,4,5,6- | 9.60* | 352.1 | B |
| | | tetrahydro-2H-[1,2′]bipyrazinyl | | | |
| 54 | 318.38 | 4-Pyridin-4-yl-6′-pyridin-3-yl-3,4,5,6-tetrahydro- | | | A |
| | | 2H-[1,2′]bipyrazinyl | | | |
| 55 | 389.50 | 6′-(5-Isopropyl-2-methoxy-phenyl)-4-pyridin-4-yl- | | | A |
| | | 3,4,5,6-tetrahydro-2H-[1,2′]bipyrazinyl | | | |
| 56 | 335.39 | 6′-(3-Fluoro-phenyl)-4-pyridin-2-yl-3,4,5,6- | 9.12* | 336.1 | A |
| | | tetrahydro-2H-[1,2′]bipyrazinyl | | | |
| 57 | 333.40 | 4-(4-Pyridin-4-yl-3,4,5,6-tetrahydro-2H- | 8.38* | 334.2 | A |
| | | [1,2′]bipyrazinyl-6′-yl)-phenol | | | |
| 58 | 364.41 | Furan-2-yl-[6′-(3-hydroxymethyl-phenyl)-2,3,5,6- | 7.08* | 365.1 | A |
| | | tetrahydro-[1,2′]bipyrazinyl-4-yl]-methanone | | | |
| 59 | 363.42 | [6′-(4-Aminomethyl-phenyl)-2,3,5,6-tetrahydro- | 7.58* | 364.2 | A |
| | | [1,2′]bipyrazinyl-4-yl]-furan-2-yl-methanone | | | |
| 60 | 405.50 | 4-[4-(3,4-Dimethoxy-phenyl)-3,4,5,6-tetrahydro- | 8.65* | 406.2 | A |
| | | 2H-[1,2′]bipyrazinyl-6′-yl]-benzylamine | | | |
| 61 | 385.47 | {4-[4-(1H-Indol-4-yl)-3,4,5,6-tetrahydro-2H- | 8.42* | 386.2 | A |
| | | [1,2′]bipyrazinyl-6′-yl]-phenyl}-methanol | | | |
| 62 | 393.85 | 2-[6′-(3-Chloro-4-fluoro-phenyl)-2,3,5,6- | | | B |
| | | tetrahydro-[1,2′]bipyrazinyl-4-yl]-benzonitrile | | | |
| 63 | 367.50 | {3-[4-(2-Pyrrolidin-1-yl-ethyl)-3,4,5,6-tetrahydro- | | | A |
| | | 2H-[1,2′]bipyrazinyl-6′-yl]-phenyl}-methanol | | | |
| 64 | 356.43 | 4-(1H-Indol-4-yl)-6′-pyridin-3-yl-3,4,5,6- | | | B |
| | | tetrahydro-2H-[1,2′]bipyrazinyl | | | |
| 65 | 365.46 | 1-[5-(4-Pyridin-4-yl-3,4,5,6-tetrahydro-2H- | 1.06 | 366 | C |
| | | [1,2′]bipyrazinyl-6′-yl)-thiophen-2-yl]-ethanone | | | |
| 66 | 363.49 | 6′-(4-Methylsulfanyl-phenyl)-4-pyridin-4-yl- | 1.16 | 364 | C |
| | | 3,4,5,6-tetrahydro-2H-[1,2′]bipyrazinyl | | | |
| 67 | 317.40 | 6′-Phenyl-4-pyridin-4-yl-3,4,5,6-tetrahydro-2H- | 1.06 | 318 | B |
| | | [1,2′]bipyrazinyl | | | |
| 68 | 359.43 | 4-(4-Phenyl-3,4,5,6-tetrahydro-2H- | 1.12 | 360 | C |
| | | [1,2′]bipyrazinyl-6′-yl)-benzamide | | | |
| 69 | 373.48 | 6′-Benzo[b]thiophen-2-yl-4-pyridin-4-yl-3,4,5,6- | 1.28 | 374 | C |
| | | tetrahydro-2H-[1,2′]bipyrazinyl | | | |
| 70 | 360.42 | 2-(4-Pyridin-4-yl-3,4,5,6-tetrahydro-2H- | 0.82 | 361 | A |
| | | [1,2′]bipyrazinyl-6′-yl)-benzamide | | | |
| 71 | 360.42 | 3-(4-Pyridin-4-yl-3,4,5,6-tetrahydro-2H- | 0.79 | 361 | B |
| | | [1,2′]bipyrazinyl-6′-yl)-benzamide | | | |
| 72 | 367.46 | 6′-Naphthalen-2-yl-4-pyridin-4-yl-3,4,5,6- | 1.24 | 368 | C |
| | | tetrahydro-2H-[1,2′]bipyrazinyl | | | |
| 73 | 382.47 | 6′-Naphthalen-2-yl-4-pyridin-4-yl-3,4,5,6- | 1.73 | 383 | C |
| | | tetrahydro-2H-[1,2′]bipyrazinyl-3′-ylamine | | | |
| 74 | 375.44 | 4-(3′-Amino-4-pyridin-4-yl-3,4,5,6-tetrahydro-2H- | 0.75 | 376 | B |
| | | [1,2′]bipyrazinyl-6′-yl)-benzamide | | | |
| 75 | 378.50 | 6′-(4-Methylsulfanyl-phenyl)-4-pyridin-4-yl- | 1.02 | 379 | C |
| | | 3,4,5,6-tetrahydro-2H-[1,2′]bipyrazinyl-3′-ylamine | | | |
| 76 | 335.39 | 6′-(4-Fluoro-phenyl)-4-pyridin-4-yl-3,4,5,6- | 1.55 | 336 | B |
| | | tetrahydro-2H-[1,2′]bipyrazinyl | | | |
| 77 | 377.45 | 6′-(3,4-Dimethoxy-phenyl)-4-pyridin-4-yl-3,4,5,6- | 1.45 | 378 | C |
| | | tetrahydro-2H-[1,2′]bipyrazinyl | | | |
| 78 | 347.42 | 6′-(3-Methoxy-phenyl)-4-pyridin-4-yl-3,4,5,6- | 1.49 | 348 | B |
| | | tetrahydro-2H-[1,2′]bipyrazinyl | | | |
| 79 | 323.42 | 4-Pyridin-4-yl-6′-thiophen-3-yl-3,4,5,6-tetrahydro- | 1.45 | 324 | B |
| | | 2H-[1,2′]bipyrazinyl | | | |
| 80 | 356.88 | 4-(3-Chloro-phenyl)-6′-thiophen-3-yl-3,4,5,6- | 1.79 | 357 | A |
| | | tetrahydro-2H-[1,2′]bipyrazinyl | | | |
| 81 | 400.91 | 4-(3-Chloro-phenyl)-6′-naphthalen-2-yl-3,4,5,6- | 1.99 | 401 | A |
| | | tetrahydro-2H-[1,2′]bipyrazinyl | | | |
| 82 | 364.88 | 4-(3-Chloro-phenyl)-6′-o-tolyl-3,4,5,6-tetrahydro- | 1.80 | 365 | A |
| | | 2H-[1,2′]bipyrazinyl | | | |
| 83 | 380.88 | {4-[4-(3-Chloro-phenyl)-3,4,5,6-tetrahydro-2H- | 1.50 | 381 | B |
| | | [1,2′]bipyrazinyl-6′-yl]-phenyl}-methanol | | | |
| 84 | 340.82 | 4-(3-Chloro-phenyl)-6′-furan-3-yl-3,4,5,6- | 1.65 | 341 | B |
| | | tetrahydro-2H-[1,2′]bipyrazinyl | | | |
| 85 | 352.46 | 4-(2-Methoxy-phenyl)-6′-thiophen-3-yl-3,4,5,6- | 1.30 | 353 | A |
| | | tetrahydro-2H-[1,2′]bipyrazinyl | | | |
| 86 | 396.50 | 4-(2-Methoxy-phenyl)-6′-naphthalen-2-yl-3,4,5,6- | 1.48 | 397 | B |
| | | tetrahydro-2H-[1,2′]bipyrazinyl | | | |
| 87 | 360.46 | 4-(2-Methoxy-phenyl)-6′-o-tolyl-3,4,5,6- | 1.38 | 361 | A |
| | | tetrahydro-2H-[1,2′]bipyrazinyl | | | |
| 88 | 402.54 | 6′-(4-tert-Butyl-phenyl)-4-(2-methoxy-phenyl)- | 1.60 | 403 | A |
| | | 3,4,5,6-tetrahydro-2H-[1,2′]bipyrazinyl | | | |
| 89 | 393.88 | 4-[4-(3-Chloro-phenyl)-3,4,5,6-tetrahydro-2H- | 1.35 | 394 | B |
| | | [1,2′]bipyrazinyl-6′-yl]-benzamide | | | |
| 90 | 336.40 | 6′-Furan-3-yl-4-(2-methoxy-phenyl)-3,4,5,6- | 1.25 | 337 | B |
| | | tetrahydro-2H-[1,2′]bipyrazinyl | | | |
| 91 | 410.91 | 6′-(5-Chloro-2-methoxy-phenyl)-4-(2-methoxy- | 1.46 | 411 | A |
| | | phenyl)-3,4,5,6-tetrahydro-2H-[1,2′]bipyrazinyl | | | |
| 92 | 390.49 | 6′-(4-Ethoxy-phenyl)-4-(2-methoxy-phenyl)- | 1.44 | 391 | A |
| | | 3,4,5,6-tetrahydro-2H-[1,2′]bipyrazinyl | | | |
| 93 | 366.47 | 6′-Naphthalen-2-yl-4-phenyl-3,4,5,6-tetrahydro- | 1.66 | 367 | B |
| | | 2H-[1,2′]bipyrazinyl | | | |
| 94 | 330.44 | 4-Phenyl-6′-o-tolyl-3,4,5,6-tetrahydro-2H- | 1.54 | 331 | A |
| | | [1,2′]bipyrazinyl | | | |
| 95 | 372.52 | 6′-(4-tert-Butyl-phenyl)-4-phenyl-3,4,5,6- | 1.79 | 373 | A |
| | | tetrahydro-2H-[1,2′]bipyrazinyl | | | |
| 96 | 410.50 | 6′-(2-Fluoro-biphenyl-4-yl)-4-phenyl-3,4,5,6- | 1.79 | 411 | A |
| | | tetrahydro-2H-[1,2′]bipyrazinyl | | | |
| 97 | 350.85 | 4-(3-Chloro-phenyl)-6′-phenyl-3,4,5,6-tetrahydro- | 1.81 | 351 | B |
| | | 2H-[1,2′]bipyrazinyl | | | |
| 98 | 380.88 | 4-(3-Chloro-phenyl)-6′-(3-methoxy-phenyl)- | 1.79 | 381 | B |
| | | 3,4,5,6-tetrahydro-2H-[1,2′]bipyrazinyl | | | |
| 99 | 368.84 | 4-(3-Chloro-phenyl)-6′-(4-fluoro-phenyl)-3,4,5,6- | 1.84 | 369 | B |
| | | tetrahydro-2H-[1,2′]bipyrazinyl | | | |
| 100 | 410.91 | 4-(3-Chloro-phenyl)-6′-(3,4-dimethoxy-phenyl)- | 1.64 | 411 | B |
| | | 3,4,5,6-tetrahydro-2H-[1,2′]bipyrazinyl | | | |
| 101 | 392.89 | 1-{3-[4-(3-Chloro-phenyl)-3,4,5,6-tetrahydro-2H- | 1.74 | 393 | B |
| | | [1,2′]bipyrazinyl-6′-yl]-phenyl}-ethanone | | | |
| 102 | 396.95 | 4-(3-Chloro-phenyl)-6′-(4-methylsulfanyl-phenyl)- | 1.89 | 397 | B |
| | | 3,4,5,6-tetrahydro-2H-[1,2′]bipyrazinyl | | | |
| 103 | 392.89 | 1-{4-[4-(3-Chloro-phenyl)-3,4,5,6-tetrahydro-2H- | 1.74 | 393 | B |
| | | [1,2′]bipyrazinyl-6′-yl]-phenyl}-ethanone | | | |
| 104 | 389.46 | 4-[4-(2-Methoxy-phenyl)-3,4,5,6-tetrahydro-2H- | 1.11 | 390 | C |
| | | [1,2′]bipyrazinyl-6′-yl]-benzamide | | | |
| 105 | 376.46 | 4-(2-Methoxy-phenyl)-6′-(3-methoxy-phenyl)- | 1.35 | 377 | B |
| | | 3,4,5,6-tetrahydro-2H-[1,2′]bipyrazinyl | | | |
| 106 | 364.43 | 6′-(4-Fluoro-phenyl)-4-(2-methoxy-phenyl)- | 1.37 | 365 | B |
| | | 3,4,5,6-tetrahydro-2H-[1,2′]bipyrazinyl | | | |
| 107 | 406.49 | 6′-(3,4-Dimethoxy-phenyl)-4-(2-methoxy-phenyl)- | 1.25 | 407 | B |
| | | 3,4,5,6-tetrahydro-2H-[1,2′]bipyrazinyl | | | |
| 108 | 392.53 | 4-(2-Methoxy-phenyl)-6′-(4-methylsulfanyl- | 1.42 | 393 | B |
| | | phenyl)-3,4,5,6-tetrahydro-2H-[1,2′]bipyrazinyl | | | |
| 109 | 316.41 | 4,6′-Diphenyl-3,4,5,6-tetrahydro-2H- | 1.45 | 317 | B |
| | | [1,2′]bipyrazinyl | | | |
| 110 | 346.44 | 6′-(3-Methoxy-phenyl)-4-phenyl-3,4,5,6- | 1.47 | 347 | B |
| | | tetrahydro-2H-[1,2′]bipyrazinyl | | | |
| 111 | 334.40 | 6′-(4-Fluoro-phenyl)-4-phenyl-3,4,5,6-tetrahydro- | 1.47 | 335 | B |
| | | 2H-[1,2′]bipyrazinyl | | | |
| 112 | 358.45 | 1-[3-(4-Phenyl-3,4,5,6-tetrahydro-2H- | 1.38 | 359 | B |
| | | [1,2′]bipyrazinyl-6′-yl)-phenyl]-ethanone | | | |
| 113 | 364.43 | 4-(2-Fluoro-phenyl)-6′-(3-methoxy-phenyl)- | 1.70 | 365 | B |
| | | 3,4,5,6-tetrahydro-2H-[1,2′]bipyrazinyl | | | |
| 114 | 352.39 | 4-(2-Fluoro-phenyl)-6′-(4-fluoro-phenyl)-3,4,5,6- | 1.73 | 353 | B |
| | | tetrahydro-2H-[1,2′]bipyrazinyl | | | |
| 115 | 376.44 | 1-{3-[4-(2-Fluoro-phenyl)-3,4,5,6-tetrahydro-2H- | 1.63 | 377 | B |
| | | [1,2′]bipyrazinyl-6′-yl]-phenyl}-ethanone | | | |
| 116 | 380.49 | 4-(2-Fluoro-phenyl)-6′-(4-methylsulfanyl-phenyl)- | 1.78 | 381 | B |
| | | 3,4,5,6-tetrahydro-2H-[1,2′]bipyrazinyl | | | |
| 117 | 346.44 | [4-(4-Phenyl-3,4,5,6-tetrahydro-2H- | 1.20 | 347 | B |
| | | [1,2′]bipyrazinyl-6′-yl)-phenyl]-methanol | | | |
| 118 | 306.37 | 6′-Furan-3-yl-4-phenyl-3,4,5,6-tetrahydro-2H- | 1.33 | 307 | B |
| | | [1,2′]bipyrazinyl | | | |
| 119 | 380.88 | 6′-(5-Chloro-2-methoxy-phenyl)-4-phenyl-3,4,5,6- | 1.61 | 381 | A |
| | | tetrahydro-2H-[1,2′]bipyrazinyl | | | |
| 120 | 362.50 | 6′-(4-Methylsulfanyl-phenyl)-4-phenyl-3,4,5,6- | 1.53 | 363 | B |
| | | tetrahydro-2H-[1,2′]bipyrazinyl | | | |
| 121 | 360.46 | 6′-(4-Ethoxy-phenyl)-4-phenyl-3,4,5,6-tetrahydro- | 1.56 | 361 | B |
| | | 2H-[1,2′]bipyrazinyl | | | |
| 122 | 340.43 | 4-(2-Fluoro-phenyl)-6′-thiophen-3-yl-3,4,5,6- | 1.69 | 341 | B |
| | | tetrahydro-2H-[1,2′]bipyrazinyl | | | |
| 123 | 384.46 | 4-(2-Fluoro-phenyl)-6′-naphthalen-2-yl-3,4,5,6- | 1,.91 | 385 | B |
| | | tetrahydro-2H-[1,2′]bipyrazinyl | | | |
| 124 | 348.43 | 4-(2-Fluoro-phenyl)-6′-o-tolyl-3,4,5,6-tetrahydro- | 1.79 | 349 | A |
| | | 2H-[1,2′]bipyrazinyl | | | |
| 125 | 364.43 | {4-[4-(2-Fluoro-phenyl)-3,4,5,6-tetrahydro-2H- | 1.42 | 365 | B |
| | | [1,2′]bipyrazinyl-6′-yl]-phenyl}-methanol | | | |
| 126 | 324.36 | 4-(2-Fluoro-phenyl)-6′-furan-3-yl-3,4,5,6- | 1.59 | 325 | B |
| | | tetrahydro-2H-[1,2′]bipyrazinyl | | | |
| 127 | 378.45 | 6′-(4-Ethoxy-phenyl)-4-(2-fluoro-phenyl)-3,4,5,6- | 1.74 | 379 | A |
| | | tetrahydro-2H-[1,2′]bipyrazinyl | | | |
| 128 | 428.49 | 6′-(2-Fluoro-biphenyl-4-yl)-4-(2-fluoro-phenyl)- | 1.96 | 429 | A |
| | | 3,4,5,6-tetrahydro-2H-[1,2′]bipyrazinyl | | | |
| 129 | 394.91 | 4-(3-Chloro-phenyl)-6′-(4-ethoxy-phenyl)-3,4,5,6- | 1.84 | 395 | B |
| | | tetrahydro-2H-[1,2′]bipyrazinyl | | | |
| 130 | 376.44 | 1-{4-[4-(2-Fluoro-phenyl)-3,4,5,6-tetrahydro-2H- | 1.64 | 377 | B |
| | | [1,2′]bipyrazinyl-6′-yl]-phenyl}-ethanone | | | |
| 131 | 342.41 | 4-(4-Pyridin-4-yl-3,4,5,6-tetrahydro-2H- | 1.21 | 343 | B |
| | | [1,2′]bipyrazinyl-6′-yl)-benzonitrile | | | |
| 132 | 359.43 | 1-[3-(4-Pyridin-4-yl-3,4,5,6-tetrahydro-2H- | 1.17 | 360 | B |
| | | [1,2′]bipyrazinyl-6′-yl)-phenyl]-ethanone | | | |
| 133 | 359.43 | 1-[4-(4-Pyridin-4-yl-3,4,5,6-tetrahydro-2H- | 1.16 | 360 | B |
| | | [1,2′]bipyrazinyl-6′-yl)-phenyl]-ethanone | | | |
| 134 | 406.49 | 4-(3,4-Dimethoxy-phenyl)-6′-(3-methoxy-phenyl)- | 1.30 | 407 | B |
| | | 3,4,5,6-tetrahydro-2H-[1,2′]bipyrazinyl | | | |
| 135 | 330.44 | 6′-Phenyl-4-p-tolyl-3,4,5,6-tetrahydro-2H- | 1.44 | 331 | A |
| | | [1,2′]bipyrazinyl | | | |
| 136 | 348.43 | 6′-(4-Fluoro-phenyl)-4-p-tolyl-3,4,5,6-tetrahydro- | 1.45 | 349 | B |
| | | 2H-[1,2′]bipyrazinyl | | | |
| 137 | 376.53 | 6′-(4-Methylsulfanyl-phenyl)-4-p-tolyl-3,4,5,6- | 1.52 | 377 | B |
| | | tetrahydro-2H-[1,2′]bipyrazinyl | | | |
| 138 | 352.39 | 4,6′-Bis-(4-fluoro-phenyl)-3,4,5,6-tetrahydro-2H- | 1.59 | 353 | A |
| | | [1,2′]bipyrazinyl | | | |
| 139 | 394.45 | 6′-(3,4-Dimethoxy-phenyl)-4-(4-fluoro-phenyl)- | 1.42 | 395 | B |
| | | 3,4,5,6-tetrahydro-2H-[1,2′]bipyrazinyl | | | |
| 140 | 380.49 | 4-(4-Fluoro-phenyl)-6′-(4-methylsulfanyl-phenyl)- | 1.66 | 381 | B |
| | | 3,4,5,6-tetrahydro-2H-[1,2′]bipyrazinyl | | | |
| 141 | 394.43 | 1-{2-[4-(2,4-Difluoro-phenyl)-3,4,5,6-tetrahydro- | 1.64 | 395 | B |
| | | 2H-[1,2′]bipyrazinyl-6′-yl]-phenyl}-ethanone | | | |
| 142 | 366.42 | 4-(2,4-Difluoro-phenyl)-6′-p-tolyl-3,4,5,6- | 1.78 | 367 | A |
| | | tetrahydro-2H-[1,2′]bipyrazinyl | | | |
| 143 | 402.45 | 4-(2,4-Dinfluoro-phenyl)-6′-naphthalen-1-yl- | 1.91 | 403 | A |
| | | 3,4,5,6-tetrahydro-2H-[1,2′]bipyrazinyl | | | |
| 144 | 412.44 | 4-(2,4-Difluoro-phenyl)-6′-(2,5-dimethoxy- | 1.67 | 413 | A |
| | | phenyl)-3,4,5,6-tetrahydro-2H-[1,2′]bipyrazinyl | | | |
| 145 | 404.83 | 6′-(3-Chloro-4-fluoro-phenyl)-4-(2,4-difluoro- | 1.94 | 405 | A |
| | | phenyl)-3,4,5,6-tetrahydro-2H-[1,2′]bipyrazinyl | | | |
| 146 | 400.48 | 1-{2-[4-(4-Acetyl-phenyl)-3,4,5,6-tetrahydro-2H- | 1.42 | 401 | A |
| | | [1,2′]bipyrazinyl-6′-yl]-phenyl}-ethanone | | | |
| 147 | 372.47 | 1-[4-(6′-m-Tolyl-2,3,5,6-tetrahydro- | 1.60 | 373 | A |
| | | [1,2′]bipyrazinyl-4-yl)-phenyl]-ethanone | | | |
| 148 | 372.47 | 1-[4-(6′-p-Tolyl-2,3,5,6-tetrahydro- | 1.61 | 373 | B |
| | | [1,2′]bipyrazinyl-4-yl)-phenyl]-ethanone | | | |
| 149 | 408.51 | 1-[4-(6′-Naphthalen-1-yl-2,3,5,6-tetrahydro- | 1.69 | 409 | A |
| | | [1,2′]bipyrazinyl-4-yl)-phenyl]-ethanone | | | |
| 150 | 386.50 | 1-{4-[6′-(2,3-Dimethyl-phenyl)-2,3,5,6-tetrahydro- | 1.62 | 387 | A |
| | | [1,2′]bipyrazinyl-4-yl]-phenyl}-ethanone | | | |
| 151 | 410.88 | 1-{4-[6′-(3-Chloro-4-fluoro-phenyl)-2,3,5,6- | 1.74 | 411 | A |
| | | tetrahydro-[1,2′]bipyrazinyl-4-yl]-phenyl}- | | | |
| | | ethanone | | | |
| 152 | 414.53 | 1-[4-(6′-Benzo[b]thiophen-3-yl-2,3,5,6-tetrahydro- | 1.67 | 415 | B |
| | | [1,2′]bipyrazinyl-4-yl)-phenyl]-ethanone | | | |
| 153 | 426.45 | 1-{2-[4-(3-Trifluoromethyl-phenyl)-3,4,5,6- | 1.74 | 427 | A |
| | | tetrahydro-2H-[1,2′]bipyrazinyl-6′-yl]-phenyl}- | | | |
| | | ethanone | | | |
| 154 | 398.43 | 6′-m-Tolyl-4-(3-trifluoromethyl-phenyl)-3,4,5,6- | 1.87 | 399 | A |
| | | tetrahydro-2H-[1,2′]bipyrazinyl | | | |
| 155 | 398.43 | 6′-p-Tolyl-4-(3-trifluoromethyl-phenyl)-3,4,5,6- | 1.91 | 399 | A |
| | | tetrahydro-2H-[1,2′]bipyrazinyl | | | |
| 156 | 412.46 | 6′-(2,3-Dimethyl-phenyl)-4-(3-trifluoromethyl- | 1.88 | 413 | A |
| | | phenyl)-3,4,5,6-tetrahydro-2H-[1,2′]bipyrazinyl | | | |
| 157 | 436.84 | 6′-(3-Chloro-4-fluoro-phenyl)-4-(3-trifluoromethyl- | 1.99 | 437 | A |
| | | phenyl)-3,4,5,6-tetrahydro-2H-[1,2′]bipyrazinyl | | | |
| 158 | 440.49 | 6′-Benzo[b]thiophen-3-yl-4-(3-trifluoromethyl- | 1.95 | 441 | A |
| | | phenyl)-3,4,5,6-tetrahydro-2H-[1,2′]bipyrazinyl | | | |
| 159 | 404.54 | 1-{4-[6′-(4-Methylsulfanyl-phenyl)-2,3,5,6- | 1.64 | 405 | C |
| | | tetrahydro-[1,2′]bipyrazinyl-4-yl]-phenyl}- | | | |
| | | ethanone | | | |
| 160 | 440.49 | 6′-Benzo[b]thiophen-2-yl-4-(3-trifluoromethyl- | 2.09 | 441 | B |
| | | phenyl)-3,4,5,6-tetrahydro-2H-[1,2′]bipyrazinyl | | | |
| 161 | 444.46 | 6′-(3,4-Dimethoxy-phenyl)-4-(3-trifluoromethyl- | 1.71 | 445 | A |
| | | phenyl)-3,4,5,6-tetrahydro-2H-[1,2′]bipyrazinyl | | | |
| 162 | 426.45 | 1-{3-[4-(3-Trifluoromethyl-phenyl)-3,4,5,6- | 1.79 | 427 | A |
| | | tetrahydro-2H-[1,2′]bipyrazinyl-6′-yl]-phenyl}- | | | |
| | | ethanone | | | |
| 163 | 409.49 | 6′-(4-Phenoxy-phenyl)-4-pyridin-4-yl-3,4,5,6- | 1.41 | 410 | B |
| | | tetrahydro-2H-[1,2′]bipyrazinyl | | | |
| 164 | 453.39 | 6′-(3,5-Bis-trifluoromethyl-phenyl)-4-pyridin-4-yl- | 1.51 | 454 | A |
| | | 3,4,5,6-tetrahydro-2H-[1,2′]bipyrazinyl | | | |
| 165 | 345.45 | 6′-(3,5-Dimethyl-phenyl)-4-pyridin-4-yl-3,4,5,6- | 1.32 | 346 | B |
| | | tetrahydro-2H-[1,2′]bipyrazinyl | | | |
| 166 | 363.49 | 6′-(2-Methylsulfanyl-phenyl)-4-pyridin-4-yl- | 1.24 | 364 | A |
| | | 3,4,5,6-tetrahydro-2H-[1,2′]bipyrazinyl | | | |
| 167 | 342.41 | 3-(4-Pyridin-4-yl-3,4,5,6-tetrahydro-2H- | 1.19 | 343 | B |
| | | [1,2′]bipyrazinyl-6′-yl)-benzonitrile | | | |
| 168 | 468.56 | 4-(3,4-Dimethoxy-phenyl)-6′-(4-phenoxy-phenyl)- | 1.54 | 469 | A |
| | | 3,4,5-6-tetrahydro-2H-[1,2′]bipyrazinyl | | | |
| 169 | 456.55 | 4-(3,4-Dimethoxy-phenyl)-6′-(6-methoxy- | 1.44 | 457 | B |
| | | naphthalen-2-yl)-3,4,5,6-tetrahydro-2H- | | | |
| | | [1,2′]bipyrazinyl | | | |
| 170 | 404.52 | 4-(3,4-Dimethoxy-phenyl)-6′-(3,5-dimethyl- | 1.42 | 405 | A |
| | | phenyl)-3,4,5,6-tetrahydro-2H-[1,2′]bipyrazinyl | | | |
| 171 | 436.52 | 4-(3,4-Dimethoxy-phenyl)-6′-(2,3-dimethoxy- | 1.29 | 437 | A |
| | | phenyl)-3,4,5,6-tetrahydro-2H-[1,2′]bipyrazinyl | | | |
| 172 | 444.46 | 4-(3,4-Dimethoxy-phenyl)-6′-(4-trifluoromethyl- | 1.45 | 445 | B |
| | | phenyl)-3,4,5,6-tetrahydro-2H-[1,2′]bipyrazinyl | | | |
| 173 | 401.47 | 3-[4-(3,4-Dimethoxy-phenyl)-3,4,5,6-tetrahydro- | 1.27 | 402 | A |
| | | 2H-[1,2′]bipyrazinyl-6′-yl]-benzonitrile | | | |
| 174 | 468.56 | 4-(3,4-Dimethoxy-phenyl)-6′-(2-phenoxy-phenyl)- | 1.47 | 469 | A |
| | | 3,4,5,6-tetrahydro-2H-[1,2′]bipyrazinyl | | | |
| 175 | 374.49 | 6′-(2-Ethoxy-phenyl)-4-p-tolyl-3,4,5,6-tetrahydro- | 1.47 | 375 | A |
| | | 2H-[1,2′]bipyrazinyl | | | |
| 176 | 373.51 | Dimethyl-[4-(4-p-tolyl-3,4,5,6-tetrahydro-2H- | 1.25 | 374 | A |
| | | [1,2′]bipyrazinyl-6′-yl)-phenyl]-amine | | | |
| 177 | 358.49 | 6′-(3,5-Dimethyl-phenyl)-4-p-tolyl-3,4,5,6- | 1.55 | 359 | A |
| | | tetrahydro-2H-[1,2′]bipyrazinyl | | | |
| 178 | 398.43 | 4-p-Tolyl-6′-(4-trifluoromethyl-phenyl)-3,4,5,6- | 1.60 | 399 | B |
| | | tetrahydro-2H-[1,2′]bipyrazinyl | | | |
| 179 | 362.45 | 6′-(3,5-Dimethyl-phenyl)-4-(4-fluoro-phenyl)- | 1.65 | 363 | A |
| | | 3,4,5,6-tetrahydro-2H-[1,2′]bipyrazinyl | | | |
| 180 | 402.40 | 4-(4-Fluoro-phenyl)-6′-(4-trifluoromethyl-phenyl)- | 1.70 | 403 | B |
| | | 3,4,5,6-tetrahydro-2H-[1,2′]bipyrazinyl | | | |
| 181 | 352.39 | 4-(4-Fluoro-phenyl)-6′-(3-fluoro-phenyl)-3,4,5,6- | 1.61 | 353 | A |
| | | tetrahydro-2H-[1,2′]bipyrazinyl | | | |
| 182 | 393.92 | {4-[4-(3-Chloro-phenyl)-3,4,5,6-tetrahydro-2H- | 1.59 | 394 | A |
| | | [1,2′]bipyrazinyl-6′-yl]-phenyl}-dimethyl-amine | | | |
| 183 | 410.91 | 4-(3-Chloro-phenyl)-6′-(2,3-dimethoxy-phenyl)- | 1.76 | 411 | A |
| | | 3,4,5,6-tetrahydro-2H-[1,2′]bipyrazinyl | | | |
| 184 | 418.85 | 4-(3-Chloro-phenyl)-6′-(4-trifluoromethyl-phenyl)- | 1.99 | 419 | A |
| | | 3,4,5,6-tetrahydro-2H-[1,2′]bipyrazinyl | | | |
| 185 | 442.95 | 4-(3-Chloro-phenyl)-6′-(2-phenoxy-phenyl)- | 1.99 | 443 | A |
| | | 3,4,5,6-tetrahydro-2H-[1,2′]bipyrazinyl | | | |
| 186 | 389.50 | {4-[4-(2-Methoxy-phenyl)-3,4,5,6-tetrahydro-2H- | 1.19 | 390 | A |
| | | [1,2′]bipyrazinyl-6′-yl]-phenyl}-dimethyl-amine | | | |
| 187 | 426.52 | 6′-(6-Methoxy-naphthalen-2-yl)-4-(2-methoxy- | 1.49 | 427 | A |
| | | phenyl)-3,4,5,6-tetrahydro-2H-[1,2′]bipyrazinyl | | | |
| 188 | 414.43 | 4-(2-Methoxy-phenyl)-6′-(4-trifluoromethyl- | 1.50 | 415 | A |
| | | phenyl)-3,4,5,6-tetrahydro-2H-[1,2′]bipyrazinyl | | | |
| 189 | 408.51 | 6′-(4-Phenoxy-phenyl)-4-phenyl-3,4,5,6- | 1.70 | 409 | A |
| | | tetrahydro-2H-[1,2′]bipyrazinyl | | | |
| 190 | 359.48 | Dimethyl-[4-(4-phenyl-3,4,5,6-tetrahydro-2H- | 1.25 | 360 | B |
| | | [1,2′]bipyrazinyl-6′-yl)-phenyl]-amine | | | |
| 191 | 452.41 | 6′-(3,5-Bis-trifluoromethyl-phenyl)-4-phenyl- | 1.80 | 453 | A |
| | | 3,4,5,6-tetrahydro-2H-[1,2′]bipyrazinyl | | | |
| 192 | 344.46 | 6′-(3,5-Dimethyl-phenyl)-4-phenyl-3,4,5,6- | 1.60 | 345 | A |
| | | tetrahydro-2H-[1,2′]bipyrazinyl | | | |
| 193 | 384.41 | 4-Phenyl-6′-(4-trifluoromethyl-phenyl)-3,4,5,6- | 1.65 | 385 | B |
| | | tetrahydro-2H-[1,2′]bipyrazinyl | | | |
| 194 | 334.40 | 6′-(3-Fluoro-phenyl)-4-phenyl-3,4,5,6-tetrahydro- | 1.50 | 335 | A |
| | | 2H-[1,2′]bipyrazinyl | | | |
| 195 | 377.47 | {4-[4-(2-Fluoro-phenyl)-3,4,5,6-tetrahydro-2H- | 1.45 | 378 | B |
| | | [1,2′]bipyrazinyl-6′-yl]-phenyl}-dimethyl-amine | | | |
| 196 | 470.40 | 6′-(3,5-Bis-trifluoromethyl-phenyl)-4-(2-fluoro- | 2.03 | 471 | A |
| | | phenyl)-3,4,5,6-tetrahydro-2H-[1,2′]bipyrazinyl | | | |
| 197 | 402.40 | 4-(2-Fluoro-phenyl)-6′-(4-trifluoromethyl-phenyl)- | 1.90 | 403 | B |
| | | 3,4,5,6-tetrahydro-2H-[1,2′]bipyrazinyl | | | |
| 198 | 352.39 | 4-(2-Fluoro-phenyl)-6′-(3-fluoro-phenyl)-3,4,5,6- | 1.78 | 353 | A |
| | | tetrahydro-2H-[1,2′]bipyrazinyl | | | |
| 199 | 402.45 | 4-(2,4-Difluoro-phenyl)-6′-naphthalen-2-yl- | 1.88 | 403 | B |
| | | 3,4,5,6-tetrahydro-2H-[1,2′]bipyrazinyl | | | |
| 200 | 382.42 | {4-[4-(2,4-Difluoro-phenyl)-3,4,5,6-tetrahydro-2H- | 1.49 | 383 | A |
| | | [1,2′]bipyrazinyl-6′-yl]-phenyl}-methanol | | | |
| 201 | 342.35 | 4-(2,4-Difluoro-phenyl)-6′-furan-3-yl-3,4,5,6- | 1.64 | 343 | B |
| | | tetrahydro-2H-[1,2′]bipyrazinyl | | | |
| 202 | 382.42 | {2-[4-(2,4-Difluoro-phenyl)-3,4,5,6-tetrahydro-2H- | 1.54 | 383 | A |
| | | [1,2′]bipyrazinyl-6′-yl]-phenyl}-methanol | | | |
| 203 | 396.44 | 4-(2,4-Difluoro-phenyl)-6′-(4-ethoxy-phenyl)- | 1.79 | 397 | A |
| | | 3,4,5,6-tetrahydro-2H-[1,2′]bipyrazinyl | | | |
| 204 | 414.56 | 1-{4-[6′-(4-tert-Butyl-phenyl)-2,3,5,6-tetrahydro- | 1.84 | 415 | A |
| | | [1,2′]bipyrazinyl-4-yl]-phenyl}-ethanone | | | |
| 205 | 348.41 | 1-[4-(6′-Furan-3-yl-2,3,5,6-tetrahydro- | 1.42 | 349 | B |
| | | [1,2′]bipyrazinyl-4-yl)-phenyl]-ethanone | | | |
| 206 | 406.46 | 1-{4-[6′-(3-Fluoro-4-methoxy-phenyl)-2,3,5,6- | 1.52 | 407 | B |
| | | tetrahydro-[1,2′]bipyrazinyl-4-yl]-phenyl}- | | | |
| | | ethanone | | | |
| 207 | 402.50 | 1-{4-[6′-(4-Ethoxy-phenyl)-2,3,5,6-tetrahydro- | 1.57 | 403 | A |
| | | [1,2′]bipyrazinyl-4-yl]-phenyl}-ethanone | | | |
| 208 | 395.46 | {4-[4-(2,4-Difluoro-phenyl)-3,4,5,6-tetrahydro-2H- | 1.52 | 396 | B |
| | | [1,2′]bipyrazinyl-6′-yl]-phenyl}-dimethyl-amine | | | |
| 209 | 380.44 | 4-(2,4-Difluoro-phenyl)-6′-(3,5-dimethyl-phenyl)- | 1.89 | 381 | A |
| | | 3,4,5,6-tetrahydro-2H-[1,2′]bipyrazinyl | | | |
| 210 | 420.39 | 4-(2,4-Difluoro-phenyl)-6′-(4-trifluoromethyl- | 1.90 | 421 | B |
| | | phenyl)-3,4,5,6-tetrahydro-2H-[1,2′]bipyrazinyl | | | |
| 211 | 370.38 | 4-(2,4-Difluoro-phenyl)-6′-(3-fluoro-phenyl)- | 1.80 | 371 | A |
| | | 3,4,5,6-tetrahydro-2H-[1,2′]bipyrazinyl | | | |
| 212 | 402.50 | 1-{4-[6′-(2-Ethoxy-phenyl)-2,3,5,6-tetrahydro- | 1.55 | 403 | A |
| | | [1,2′]bipyrazinyl-4-yl]-phenyl}-ethanone | | | |
| 213 | 438.53 | 1-{4-[6′-(6-Methoxy-naphthalen-2-yl)-2,3,5,6- | 1.65 | 439 | B |
| | | tetrahydro-[1,2′]bipyrazinyl-4-yl]-phenyl}- | | | |
| | | ethanone | | | |
| 214 | 376.44 | 1-{4-[6′-(3-Fluoro-phenyl)-2,3,5,6-tetrahydro- | 1.58 | 377 | A |
| | | [1,2′]bipyrazinyl-4-yl]-phenyl}-ethanone | | | |
| 215 | 450.55 | 1-{4-[6′-(2-Phenoxy-phenyl)-2,3,5,6-tetrahydro- | 1.73 | 451 | A |
| | | [1,2′]bipyrazinyl-4-yl]-phenyl}-ethanone | | | |
| 216 | 427.48 | Dimethyl-{4-[4-(3-trifluoromethyl-phenyl)-3,4,5,6- | 1.58 | 428 | A |
| | | tetrahydro-2H-[1,2′]bipyrazinyl-6′-yl]-phenyl}- | | | |
| | | amine | | | |
| 217 | 476.51 | 6′-(2-Phenoxy-phenyl)-4-(3-trifluoromethyl- | 1.95 | 477 | A |
| | | phenyl)-3,4,5,6-tetrahydro-2H-[1,2′]bipyrazinyl | | | |
| 218 | 359.43 | 1-[2-(4-Pyridin-4-yl-3,4,5,6-tetrahydro-2H- | 1.14 | 360 | A |
| | | [1,2′]bipyrazinyl-6′-yl)-phenyl]-ethanone | | | |
| 219 | 331.42 | 4-Pyridin-4-yl-6′-m-tolyl-3,4,5,6-tetrahydro-2H- | 1.26 | 332 | B |
| | | [1,2′]bipyrazinyl | | | |
| 220 | 331.42 | 4-Pyridin-4-yl-6′-p-tolyl-3,4,5,6-tetrahydro-2H- | 1.24 | 332 | B |
| | | [1,2′]bipyrazinyl | | | |
| 221 | 367.46 | 6′-Naphthalen-1-yl-4-pyridin-4-yl-3,4,5,6- | 1.31 | 368 | B |
| | | tetrahydro-2H-[1,2′]bipyrazinyl | | | |
| 222 | 373.48 | 6′-Benzo[b]thiophen-3-yl-4-pyridin-4-yl-3,4,5,6- | 1.31 | 374 | B |
| | | tetrahydro-2H-[1,2′]bipyrazinyl | | | |
| 223 | 390.49 | 4-(3,4-Dimethoxy-phenyl)-6′-m-tolyl-3,4,5,6- | 1.37 | 391 | B |
| | | tetrahydro-2H-[1,2′]bipyrazinyl | | | |
| 224 | 390.49 | 4-(3,4-Dimethoxy-phenyl)-6′-p-tolyl-3,4,5,6- | 1.37 | 391 | B |
| | | tetrahydro-2H-[1,2′]bipyrazinyl | | | |
| 225 | 436.52 | 4-(3,4-Dimethoxy-phenyl)-6′-(2,5-dimethoxy- | 1.29 | 437 | A |
| | | phenyl)-3,4,5,6-tetrahydro-2H-[1,2′]bipyrazinyl | | | |
| 226 | 404.52 | 4-(3,4-Dimethoxy-phenyl)-6′-(2,3-dimethyl- | 1.39 | 405 | A |
| | | phenyl)-3,4,5,6-tetrahydro-2H-[1,2′]bipyrazinyl | | | |
| 227 | 432.55 | 6′-Benzo[b]thiophen-3-yl-4-(3,4-dimethoxy- | 1.42 | 433 | B |
| | | phenyl)-3,4,5,6-tetrahydro-2H-[1,2′]bipyrazinyl | | | |
| 228 | 344.46 | 6′-m-Tolyl-4-p-tolyl-3,4,5,6-tetrahydro-2H- | 1.52 | 345 | A |
| | | [1,2′]bipyrazinyl | | | |
| 229 | 344.46 | 4,6′-Di-p-tolyl-3,4,5,6-tetrahydro-2H- | 1.50 | 345 | A |
| | | [1,2′]bipyrazinyl | | | |
| 230 | 380.50 | 6′-Naphthalen-1-yl-4-p-tolyl-3,4,5,6-tetrahydro- | 1.52 | 381 | A |
| | | 2H-[1,2′]bipyrazinyl | | | |
| 231 | 386.52 | 6′-Benzo[b]thiophen-3-yl-4-p-tolyl-3,4,5,6- | 1.57 | 387 | A |
| | | tetrahydro-2H-[1,2′]bipyrazinyl | | | |
| 232 | 348.43 | 4-(4-Fluoro-phenyl)-6′-m-tolyl-3,4,5,6-tetrahydro- | 1.60 | 349 | A |
| | | 2H-[1,2′]bipyrazinyl | | | |
| 233 | 348.43 | 4-(4-Fluoro-phenyl)-6′-p-tolyl-3,4,5,6-tetrahydro- | 1.58 | 349 | B |
| | | 2H-[1,2′]bipyrazinyl | | | |
| 234 | 394.45 | 6′-(2,5-Dimethoxy-phenyl)-4-(4-fluoro-phenyl)- | 1.45 | 395 | A |
| | | 3,4,5,6-tetrahydro-2H-[1,2′]bipyrazinyl | | | |
| 235 | 390.49 | 6′-Benzo[b]thiophen-3-yl-4-(4-fluoro-phenyl)- | 1.68 | 391 | B |
| | | 3,4,5,6-tetrahydro-2H-[1,2′]bipyrazinyl | | | |
| 236 | 392.89 | 1-{2-[4-(3-Chloro-phenyl)-3,4,5,6-tetrahydro-2H- | 1.65 | 393 | A |
| | | [1,2′]bipyrazinyl-6′-yl]-phenyl}-ethanone | | | |
| 237 | 364.88 | 4-(3-Chloro-phenyl)-6′-p-tolyl-3,4,5,6-tetrahydro- | 1.85 | 365 | A |
| | | 2H-[1,2′]bipyrazinyl | | | |
| 238 | 403.29 | 6′-(3-Chloro-4-fluoro-phenyl)-4-(3-chloro-phenyl)- | 2.01 | 403 | A |
| | | 3,4,5,6-tetrahydro-2H-[1,2′]bipyrazinyl | | | |
| 239 | 406.94 | 6′-Benzo[b]thiophen-3-yl-4-(3-chloro-phenyl)- | 1.99 | 407 | A |
| | | 3,4,5,6-tetrahydro-2H-[1,2′]bipyrazinyl | | | |
| 240 | 388.47 | 1-{2-[4-(2-Methoxy-phenyl)-3,4,5,6-tetrahydro- | 1.26 | 389 | A |
| | | 2H-[1,2′]bipyrazinyl-6′-yl]-phenyl}-ethanone | | | |
| 241 | 360.46 | 4-(2-Methoxy-phenyl)-6′-p-tolyl-3,4,5,6- | 1.44 | 361 | A |
| | | tetrahydro-2H-[1,2′]bipyrazinyl | | | |
| 242 | 398.87 | 6′-(3-Chloro-4-fluoro-phenyl)-4-(2-methoxy- | 1.49 | 399 | A |
| | | phenyl)-3,4,5,6-tetrahydro-2H-[1,2′]bipyrazinyl | | | |
| 243 | 402.52 | 6′-Benzo[b]thiophen-3-yl-4-(2-methoxy-phenyl)- | 1.47 | 403 | A |
| | | 3,4,5,6-tetrahydro-2H-[1,2′]bipyrazinyl | | | |
| 244 | 330.44 | 4-Phenyl-6′-m-tolyl-3,4,5,6-tetrahydro-2H- | 1.54 | 331 | A |
| | | [1,2′]bipyrazinyl | | | |
| 245 | 330.44 | 4-Phenyl-6′-p-tolyl-3,4,5,6-tetrahydro-2H- | 1.54 | 331 | B |
| | | [1,2′]bipyrazinyl | | | |
| 246 | 366.47 | 6′-Naphthalen-1-yl-4-phenyl-3,4,5,6-tetrahydro- | 1.59 | 367 | A |
| | | 2H-[1,2′]bipyrazinyl | | | |
| 247 | 372.50 | 6′-Benzo[b]thiophen-3-yl-4-phenyl-3,4,5,6- | 1.60 | 373 | B |
| | | tetrahydro-2H-[1,2′]bipyrazinyl | | | |
| 248 | 348.43 | 4-(2-Fluoro-phenyl)-6′-p-tolyl-3,4,5,6-tetrahydro- | 1.77 | 349 | A |
| | | 2H-[1,2′]bipyrazinyl | | | |
| 249 | 384.46 | 4-(2-Fluoro-phenyl)-6′-naphthalen-1-yl-3,4,5,6- | 1.82 | 385 | A |
| | | tetrahydro-2H-[1,2′]bipyrazinyl | | | |
| 250 | 362.45 | 6′-(2,3-Dimethyl-phenyl)-4-(2-fluoro-phenyl)- | 1.77 | 363 | A |
| | | 3,4,5,6-tetrahydro-2H-[1,2′]bipyrazinyl | | | |
| 251 | 390.49 | 6′-Benzo[b]thiophen-3-yl-4-(2-fluoro-phenyl)- | 1.85 | 391 | A |
| | | 3,4,5,6-tetrahydro-2H-[1,2′]bipyrazinyl | | | |
| 252 | 434.47 | 6′-Naphthalen-2-yl-4-(3-trifluoromethyl-phenyl)- | 1.95 | 435 | A |
| | | 3,4,5,6-tetrahydro-2H-[1,2′]bipyrazinyl | | | |
| 253 | 440.52 | 6′-(4-tert-Butyl-phenyl)-4-(3-trifluoromethyl- | 2.03 | 441 | A |
| | | phenyl)-3,4,5,6-tetrahydro-2H-[1,2′]bipyrazinyl | | | |
| 254 | 414.43 | {4-[4-(3-Trifluoromethyl-phenyl)-3,4,5,6- | 1.53 | 415 | A |
| | | tetrahydro-2H-[1,2′]bipyrazinyl-6′-yl]-phenyl}- | | | |
| | | methanol | | | |
| 255 | 374.37 | 6′-Furan-3-yl-4-(3-trifluoromethyl-phenyl)-3,4,5,6- | 1.70 | 375 | A |
| | | tetrahydro-2H-[1,2′]bipyrazinyl | | | |
| 256 | 331.42 | 4-Pyridin-4-yl-6′-o-tolyl-3,4,5,6-tetrahydro-2H- | 1.23 | 332 | A |
| | | [1,2′]bipyrazinyl | | | |
| 257 | 373.51 | 6′-(4-tert-Butyl-phenyl)-4-pyridin-4-yl-3,4,5,6- | 1.40 | 374 | B |
| | | tetrahydro-2H-[1,2′]bipyrazinyl | | | |
| 258 | 347.42 | [4-(4-Pyridin-4-yl-3,4,5,6-tetrahydro-2H- | 1.04 | 348 | B |
| | | [1,2′]bipyrazinyl-6′-yl)-phenyl]-methanol | | | |
| 259 | 381.87 | 6′-(5-Chloro-2-methoxy-phenyl)-4-pyridin-4-yl- | 1.29 | 382 | A |
| | | 3,4,5,6-tetrahydro-2H-[1,2′]bipyrazinyl | | | |
| 260 | 361.45 | 6′-(4-Ethoxy-phenyl)-4-pyridin-4-yl-3,4,5,6- | 1.24 | 362 | B |
| | | tetrahydro-2H-[1,2′]bipyrazinyl | | | |
| 261 | 382.49 | 4-(3,4-Dimethoxy-phenyl)-6′-thiophen-3-yl- | 1.29 | 383 | B |
| | | 3,4,5,6-tetrahydro-2H-[1,2′]bipyrazinyl | | | |
| 262 | 390.49 | 4-(3,4-Dimethoxy-phenyl)-6′-o-tolyl-3,4,5,6- | 1.36 | 391 | A |
| | | tetrahydro-2H-[1,2′]bipyrazinyl | | | |
| 263 | 432.57 | 6′-(4-tert-Butyl-phenyl)-4-(3,4-dimethoxy-phenyl)- | 1.54 | 433 | A |
| | | 3,4,5,6-tetrahydro-2H-[1,2′]bipyrazinyl | | | |
| 264 | 406.49 | {2-[4-(3,4-Dimethoxy-phenyl)-3,4,5,6-tetrahydro- | 1.17 | 407 | A |
| | | 2H-[1,2′]bipyrazinyl-6′-yl]-phenyl}-methanol | | | |
| 265 | 378.45 | 6′-(3-Fluoro-4-methoxy-phenyl)-4-p-tolyl-3,4,5,6- | 1.44 | 379 | A |
| | | tetrahydro-2H-[1,2′]bipyrazinyl | | | |
| 266 | 374.49 | 6′-(4-Ethoxy-phenyl)-4-p-tolyl-3,4,5,6-tetrahydro- | 1.50 | 375 | A |
| | | 2H-[1,2′]bipyrazinyl | | | |
| 267 | 384.46 | 4-(4-Fluoro-phenyl)-6′-naphthalen-2-yl-3,4,5,6- | 1.70 | 385 | C |
| | | tetrahydro-2H-[1,2′]bipyrazinyl | | | |
| 268 | 428.49 | 6′-(2-Fluoro-biphenyl-4-yl)-4-(4-fluoro-phenyl)- | 1.82 | 429 | A |
| | | 3,4,5,6-tetrahydro-2H-[1,2′]bipyrazinyl | | | |
| 269 | 364.43 | {4-[4-(4-Fluoro-phenyl)-3,4,5,6-tetrahydro-2H- | 1.27 | 365 | B |
| | | [1,2′]bipyrazinyl-6′-yl]-phenyl}-methanol | | | |
| 270 | 324.36 | 4-(4-Fluoro-phenyl)-6′-furan-3-yl-3,4,5,6- | 1.40 | 325 | A |
| | | tetrahydro-2H-[1,2′]bipyrazinyl | | | |
| 271 | 378.45 | 6′-(4-Ethoxy-phenyl)-4-(4-fluoro-phenyl)-3,4,5,6- | 1.55 | 379 | A |
| | | tetrahydro-2H-[1,2′]bipyrazinyl | | | |
| 272 | 408.48 | 6′-Benzo[b]thiophen-2-yl-4-(2,4-difluoro-phenyl)- | 1.98 | 409 | B |
| | | 3,4,5,6-tetrahydro-2H-[1,2′]bipyrazinyl | | | |
| 273 | 394.43 | 1-{3-[4-(2,4-Difluoro-phenyl)-3,4,5,6-tetrahydro- | 1.65 | 395 | A |
| | | 2H-[1,2′]bipyrazinyl-6′-yl]-phenyl}-ethanone | | | |
| 274 | 398.48 | 4-(2,4-Difluoro-phenyl)-6′-(4-methylsulfanyl- | 1.80 | 399 | B |
| | | phenyl)-3,4,5,6-tetrahydro-2H-[1,2′]bipyrazinyl | | | |
| 275 | 394.43 | 1-{4-[4-(2,4-Difluoro-phenyl)-3,4,5,6-tetrahydro- | 1.68 | 395 | B |
| | | 2H-[1,2′]bipyrazinyl-6′-yl]-phenyl}-ethanone | | | |
| 276 | 376.44 | 1-{4-[6′-(4-Fluoro-phenyl)-2,3,5,6-tetrahydro- | 1.55 | 377 | B |
| | | [1,2′]bipyrazinyl-4-yl]-phenyl}-ethanone | | | |
| 277 | 305.38 | 2-Furan-3-yl-6-(4-phenyl-piperidin-1-yl)-pyrazine | 1.68 | 306 | A |
| 278 | 363.44 | 2-(3-Fluoro-4-methoxy-phenyl)-6-(4-phenyl- | 1.82 | 364 | A |
| | | piperidin-1-yl)-pyrazine | | | |
| 279 | 400.55 | 6′-Benzo[b]thiophen-3-yl-3-methyl-4-m-tolyl- | 1.55 | 401 | A |
| | | 3,4,5,6-tetrahydro-2H-[1,2′]bipyrazinyl | | | |
| 280 | 358.49 | Dimethyl-{4-[6-(4-phenyl-piperidin-1-yl)-pyrazin- | 1.60 | 359 | B |
| | | 2-yl]-phenyl}-amine | | | |
| 281 | 396.90 | 6′-(3-Chloro-4-fluoro-phenyl)-4-(2,4-dimethyl- | 1.96 | 397 | A |
| | | phenyl)-3,4,5,6-tetrahydro-2H-[1,2′]bipyrazinyl | | | |
| 282 | 386.46 | 6′-Benzofuran-2-yl-4-(4-methoxy-phenyl)-3,4,5,6- | 1.53 | 387 | B |
| | | tetrahydro-2H-[1,2′]bipyrazinyl | | | |
| 283 | 402.52 | 6′-Benzo[b]thiophen-3-yl-4-(4-methoxy-phenyl)- | 1.47 | 403 | B |
| | | 3,4,5,6-tetrahydro-2H-[1,2′]bipyrazinyl | | | |
| 284 | 360.46 | 4-(3-Methoxy-phenyl)-6′-p-tolyl-3,4,5,6- | 1.61 | 361 | B |
| | | tetrahydro-2H-[1,2′]bipyrazinyl | | | |
| 285 | 386.46 | 6′-Benzofuran-2-yl-4-(3-methoxy-phenyl)-3,4,5,6- | 1.78 | 387 | B |
| | | tetrahydro-2H-[1,2′]bipyrazinyl | | | |
| 286 | 402.52 | 6′-Benzo[b]thiophen-3-yl-4-(3-methoxy-phenyl)- | 1.75 | 403 | B |
| | | 3,4,5,6-tetrahydro-2H-[1,2′]bipyrazinyl | | | |
| 287 | 435.36 | 4-(3,4-Dichloro-phenyl)-6′-naphthalen-1-yl- | 2.12 | 435 | A |
| | | 3,4,5,6-tetrahydro-2H-[1,2′]bipyrazinyl | | | |
| 288 | 404.52 | 6′-(3,4-Dimethoxy-phenyl)-4-(2,4-dimethyl- | 1.63 | 405 | A |
| | | phenyl)-3,4,5,6-tetrahydro-2H[1,2′]bipyrazinyl | | | |
| 289 | 392.53 | 1-{5-[4-(2,4-Dimethyl-phenyl)-3,4,5,6-tetrahydro- | 1.71 | 393 | A |
| | | 2H-[1,2′]bipyrazinyl-6′-yl]-thiophen-2-yl}- | | | |
| | | ethanone | | | |
| 290 | 389.46 | 4-[4-(4-Methoxy-phenyl)-3,4,5,6-tetrahydro-2H- | 1.12 | 390 | C |
| | | [1,2′]bipyrazinyl-6′-yl]-benzamide | | | |
| 291 | 346.44 | 4-(4-Methoxy-phenyl)-6′-phenyl-3,4,5,6- | 1.34 | 347 | A |
| | | tetrahydro-2H-[1,2′]bipyrazinyl | | | |
| 292 | 402.52 | 6′-Benzo[b]thiophen-2-yl-4-(4-methoxy-phenyl)- | 1.58 | 403 | B |
| | | 3,4,5,6-tetrahydro-2H-[1,2′]bipyrazinyl | | | |
| 293 | 371.45 | 4-[4-(4-Methoxy-phenyl)-3,4,5,6-tetrahydro-2H- | 1.35 | 372 | B |
| | | [1,2′]bipyrazinyl-6′-yl]-benzonitrile | | | |
| 294 | 376.46 | 4-(4-Methoxy-phenyl)-6′-(3-methoxy-phenyl)- | 1.37 | 377 | A |
| | | 3,4,5,6-tetrahydro-2H-[1,2′]bipyrazinyl | | | |
| 295 | 400.55 | 6′-Benzo[b]thiophen-2-yl-4-(2,3-dimethyl-phenyl)- | 2.17 | 401 | A |
| | | 3,4,5,6-tetrahydro-2H-[1,2′]bipyrazinyl | | | |
| 296 | 392.53 | 1-{5-[4-(2,3-Dimethyl-phenyl)-3,4,5,6-tetrahydro- | 1.80 | 393 | A |
| | | 2H-[1,2′]bipyrazinyl-6′-yl]-thiophen-2-yl}- | | | |
| | | ethanone | | | |
| 297 | 386.50 | 1-{3-[4-(2,3-Dimethyl-phenyl)-3,4,5,6-tetrahydro- | 1.78 | 387 | A |
| | | 2H-[1,2′]bipyrazinyl-6′-yl]-phenyl}-ethanone | | | |
| 298 | 386.50 | 1-{4-[4-(2,3-Dimethyl-phenyl)-3,4,5,6-tetrahydro- | 1.80 | 387 | A |
| | | 2H-[1,2′]bipyrazinyl-6′-yl]-phenyl}-ethanone | | | |
| 299 | 387.49 | 4-(3-Methyl-4-m-tolyl-3,4,5,6-tetrahydro-2H- | 1.15 | 388 | B |
| | | [1,2′]bipyrazinyl-6′-yl)-benzamide | | | |
| 300 | 400.55 | 6′-Benzo[b]thiophen-2-yl-3-methyl-4-m-tolyl- | 1.59 | 401 | A |
| | | 3,4,5,6-tetrahydro-2H-[1,2′]bipyrazinyl | | | |
| 301 | 390.55 | 3-Methyl-6′-(4-methylsulfanyl-phenyl)- | 1.47 | 391 | A |
| | | 4-m-tolyl-3,4,5,6-tetrahydro-2H- | | | |
| | | [1,2′]bipyrazinyl | | | |
| 302 | 380.88 | 4-(4-Chloro-phenyl)-6′-(3-methoxy-phenyl)- | 1.81 | 381 | B |
| | | 3,4,5,6-tetrahydro-2H-[1,2′]bipyrazinyl | | | |
| 303 | 342.41 | 4-(4-Pyridin-2-yl-3,4,5,6-tetrahydro-2H- | 1.22 | 343 | B |
| | | [1,2′]bipyrazinyl-6′-yl)-benzonitrile | | | |
| 304 | 347.42 | 6′-(3-Methoxy-phenyl)-4-pyridin-2-yl-3,4,5,6- | 1.22 | 348 | B |
| | | tetrahydro-2H-[1,2′]bipyrazinyl | | | |
| 305 | 335.39 | 6′-(4-Fluoro-phenyl)-4-pyridin-2-yl-3,4,5,6- | 1.22 | 336 | B |
| | | tetrahydro-2H-[1,2′]bipyrazinyl | | | |
| 306 | 377.45 | 6′-(3,4-Dimethoxy-phenyl)-4-pyridin-2-yl-3,4,5,6- | 1.11 | 378 | B |
| | | tetrahydro-2H-[1,2′]bipyrazinyl | | | |
| 307 | 359.43 | 1-[3-(4-Pyridin-2-yl-3,4,5,6-tetrahydro-2H- | 1.17 | 360 | B |
| | | [1,2′]bipyrazinyl-6′-yl)-phenyl]-ethanone | | | |
| 308 | 359.43 | 1-[4-(4-Pyridin-2-yl-3,4,5,6-tetrahydro-2H- | 1.14 | 360 | B |
| | | [1,2′]bipyrazinyl-6′-yl)-phenyl]-ethanone | | | |
| 309 | 402.40 | 6′-(4-Fluoro-phenyl)-4-(4-trifluoromethyl-phenyl)- | 1.88 | 403 | A |
| | | 3,4,5,6-tetrahydro-2H-[1,2′]bipyrazinyl | | | |
| 310 | 444.46 | 6′-(3,4-Dimethoxy-phenyl)-4-(4-trifluoromethyl- | 1.76 | 445 | A |
| | | phenyl)-3,4,5,6-tetrahydro-2H-[1,2′]bipyrazinyl | | | |
| 311 | 430.50 | 6′-(4-Methylsulfanyl-phenyl)-4-(4-trifluoromethyl- | 1.98 | 431 | B |
| | | phenyl)-3,4,5,6-tetrahydro-2H-[1,2′]bipyrazinyl | | | |
| 312 | 360.42 | 1-[3-(2′,3′,5′,6′-Tetrahydro-[2,1′;4′,2″]terpyrazin-6- | 1.38 | 361 | B |
| | | yl)-phenyl]-ethanone | | | |
| 313 | 397.28 | 6-(4-Bromo-phenyl)-2′,3′,5′,6′-tetrahydro- | 1.66 | 399 | C |
| | | [2,1′;4′,2″]terpyrazine | | | |
| 314 | 364.48 | 6-(4-Methylsulfanyl-phenyl)-2′,3′,5′,6′-tetrahydro- | 1.51 | 365 | B |
| | | [2,1′;4′,2″]terpyrazine | | | |
| 315 | 415.33 | {4-[4-(3,4-Dichloro-phenyl)-3,4,5,6-tetrahydro- | 1.67 | 415 | B |
| | | 2H-[1,2′]bipyrazinyl-6′-yl]-phenyl}-methanol | | | |
| 316 | 396.90 | 6′-(3-Chloro-4-fluoro-phenyl)-4-(3,4-dimethyl- | 1.65 | 397 | A |
| | | phenyl)-3,4,5,6-tetrahydro-2H-[1,2′]bipyrazinyl | | | |
| 317 | 400.55 | 6′-Benzo[b]thiophen-3-yl-4-(3,4-dimethyl-phenyl)- | 1.63 | 401 | A |
| | | 3,4,5,6-tetrahydro-2H-[1,2′]bipyrazinyl | | | |
| 318 | 365.48 | 2-Naphthalen-1-yl-6-(4-phenyl-piperidin-1-yl)- | 1.92 | 366 | A |
| | | pyrazine | | | |
| 319 | 371.51 | 2-Benzo[b]thiophen-3-yl-6-(4-phenyl-piperidin-1- | 1.96 | 372 | A |
| | | yl)-pyrazine | | | |
| 320 | 394.52 | 4-(2,4-Dimethyl-phenyl)-6′-naphthalen-2-yl- | 1.96 | 395 | A |
| | | 3,4,5,6-tetrahydro-2H-[1,2′]bipyrazinyl | | | |
| 321 | 374.49 | {4-[4-(2,4-Dimethyl-phenyl)-3,4,5,6-tetrahydro- | 1.45 | 375 | A |
| | | 2H-[1,2′]bipyrazinyl-6′-yl]-phenyl}-methanol | | | |
| 322 | 396.50 | 4-(3-Methoxy-phenyl)-6′-naphthalen-2-yl-3,4,5,6- | 1.73 | 397 | B |
| | | tetrahydro-2H-[1,2′]bipyrazinyl | | | |
| 323 | 376.46 | {4-[4-(3-Methoxy-phenyl)-3,4,5,6-tetrahydro-2H- | 1.27 | 377 | B |
| | | [1,2′]bipyrazinyl-6′-yl]-phenyl}-methanol | | | |
| 324 | 336.40 | 6′-Furan-3-yl-4-(3-methoxy-phenyl)-3,4,5,6- | 1.46 | 337 | A |
| | | tetrahydro-2H-[1,2′]bipyrazinyl | | | |
| 325 | 364.88 | 4-(4-Chloro-phenyl)-6′-m-tolyl-3,4,5,6-tetrahydro- | 1.92 | 365 | A |
| | | 2H-[1,2′]bipyrazinyl | | | |
| 326 | 406.94 | 6′-Benzo[b]thiophen-3-yl-4-(4-chloro-phenyl)- | 1.99 | 407 | B |
| | | 3,4,5,6-tetrahydro-2H-[1,2′]bipyrazinyl | | | |
| 327 | 331.42 | 4-Pyridin-2-yl-6′-m-tolyl-3,4,5,6-tetrahydro-2H- | 1.30 | 332 | A |
| | | [1,2′]bipyrazinyl | | | |
| 328 | 331.42 | 4-Pyridin-2-yl-6′-p-tolyl-3,4,5,6-tetrahydro-2H- | 1.28 | 332 | B |
| | | [1,2′]bipyrazinyl | | | |
| 329 | 332.41 | 6-m-Tolyl-2′,3′,5′,6′-tetrahydro- | 1.44 | 333 | B |
| | | [2,1′;4′,2″]terpyrazine | | | |
| 330 | 332.41 | 6-p-Tolyl-2′,3′,5′,6′-tetrahydro- | 1.44 | 333 | B |
| | | [2,1′;4′,2″]terpyrazine | | | |
| 331 | 368.44 | 6-Naphthalen-1-yl-2′,3′,5′,6′-tetrahydro- | 1.50 | 369 | A |
| | | [2,1′;4′,2″]terpyrazine | | | |
| 332 | 374.47 | 6-Benzo[b]thiophen-3-yl-2′,3′,5′,6′-tetrahydro- | 1.48 | 375 | C |
| | | [2,1′;4′,2″]terpyrazine | | | |
| 333 | 374.49 | 6-(4-tert-Butyl-phenyl)-2′,3′,5′,6′-tetrahydro- | 1.64 | 375 | A |
| | | [2,1′;4′,2″]terpyrazine | | | |
| 334 | 412.47 | 6-(2-Fluoro-biphenyl-4-yl)-2′,3′,5′,6′-tetrahydro- | 1.68 | 413 | A |
| | | [2,1′;4′,2″]terpyrazine | | | |
| 335 | 394.52 | 4-(3,4-Dimethyl-phenyl)-6′-naphthalen-2-yl- | 1.66 | 395 | B |
| | | 3,4,5,6-tetrahydro-2H-[1,2′]bipyrazinyl | | | |
| 336 | 321.45 | 2-(4-Phenyl-piperidin-1-yl)-6-thiophen-3-yl- | 1.78 | 322 | B |
| | | pyrazine | | | |
| 337 | 365.48 | 2-Naphthalen-2-yl-6-(4-phenyl-piperidin-1-yl)- | 2.02 | 366 | B |
| | | pyrazine | | | |
| 338 | 372.52 | 4-(2,3-Dimethyl-phenyl)-6′-(3,5-dimethyl-phenyl)- | 2.04 | 373 | A |
| | | 3,4,5,6-tetrahydro-2H-[1,2′]bipyrazinyl | | | |
| 339 | 372.52 | 6′-(3,5-Dimethyl-phenyl)-3-methyl-4-m-tolyl- | 1.55 | 373 | A |
| | | 3,4,5,6-tetrahydro-2H-[1,2′]bipyrazinyl | | | |
| 340 | 393.92 | {4-[4-(4-Chloro-phenyl)-3,4,5,6-tetrahydro-2H- | 1.60 | 394 | A |
| | | [1,2′]bipyrazinyl-6′-yl]-phenyl}-dimethyl-amine | | | |
| 341 | 409.49 | 6′-(4-Phenoxy-phenyl)-4-pyridin-2-yl-3,4,5,6- | 1.47 | 410 | A |
| | | tetrahydro-2H-[1,2′]bipyrazinyl | | | |
| 342 | 363.49 | 6′-(2-Methylsulfanyl-phenyl)-4-pyridin-2-yl- | 1.29 | 364 | A |
| | | 3,4,5,6-tetrahydro-2H-[1,2′]bipyrazinyl | | | |
| 343 | 361.45 | Dimethyl-[4-(2′,3′,5′,6′-tetrahydro- | 1.15 | 362 | B |
| | | [2,1′;4′,2″]terpyrazin-6-yl)-phenyl]-amine | | | |
| 344 | 346.44 | 6-(3,5-Dimethyl-phenyl)-2′,3′,5′,6′-tetrahydro- | 1.49 | 347 | A |
| | | [2,1′;4′,2″]terpyrazine | | | |
| 345 | 386.38 | 6-(4-Trifluoromethyl-phenyl)-2′,3′,5′,6′-tetrahydro- | 1.58 | 387 | B |
| | | [2,1′;4′,2″]terpyrazine | | | |
| 346 | 334.42 | 4-(2,3-Dimethyl-phenyl)-6′-furan-3-yl-3,4,5,6- | 1.76 | 335 | A |
| | | tetrahydro-2H-[1,2′]bipyrazinyl | | | |
| 347 | 394.52 | 3-Methyl-6′-naphthalen-2-yl-4-m-tolyl-3,4,5,6- | 1.56 | 395 | A |
| | | tetrahydro-2H-[1,2′]bipyrazinyl | | | |
| 348 | 400.91 | 4-(4-Chloro-phenyl)-6′-naphthalen-2-yl-3,4,5,6- | 2.02 | 401 | B |
| | | tetrahydro-2H-[1,2′]bipyrazinyl | | | |
| 349 | 340.82 | 4-(4-Chloro-phenyl)-6′-furan-3-yl-3,4,5,6- | 1.70 | 341 | A |
| | | tetrahydro-2H-[1,2′]bipyrazinyl | | | |
| 350 | 323.42 | 4-Pyridin-2-yl-6′-thiophen-3-yl-3,4,5,6-tetrahydro- | 1.17 | 324 | B |
| | | 2H-[1,2′]bipyrazinyl | | | |
| 351 | 347.42 | [4-(4-Pyridin-2-yl-3,4,5,6-tetrahydro-2H- | 1.03 | 348 | B |
| | | [1,2′]bipyrazinyl-6′-yl)-phenyl]-methanol | | | |
| 352 | 434.47 | 6′-Naphthalen-2-yl-4-(4-trifluoromethyl-phenyl)- | 2.03 | 435 | B |
| | | 3,4,5,6-tetrahydro-2H-[1,2′]bipyrazinyl | | | |
| 353 | 374.37 | 6′-Furan-3-yl-4-(4-trifluoromethyl-phenyl)-3,4,5,6- | 1.77 | 375 | A |
| | | tetrahydro-2H-[1,2′]bipyrazinyl | | | |
| 354 | 412.46 | 4-(2,4-Dimethyl-phenyl)-6′-(4-trifluoromethyl- | 1.98 | 413 | A |
| | | phenyl)-3,4,5,6-tetrahydro-2H-[1,2′]bipyrazinyl | | | |
| 355 | 414.43 | 4-(4-Methoxy-phenyl)-6′-(4-trifluoromethyl- | 1.55 | 415 | B |
| | | phenyl)-3,4,5,6-tetrahydro-2H-[1,2′]bipyrazinyl | | | |
| 356 | 414.43 | 4-(3-Methoxy-phenyl)-6′-(4-trifluoromethyl- | 1.80 | 415 | B |
| | | phenyl)-3,4,5,6-tetrahydro-2H-[1,2′]bipyrazinyl | | | |
| 357 | 428.37 | {4-[4-(3,4-Dichloro-phenyl)-3,4,5,6-tetrahydro- | 1.74 | 428 | A |
| | | 2H-[1,2′]bipyrazinyl-6′-yl]-phenyl}-dimethyl-amine | | | |
| 358 | 413.35 | 4-(3,4-Dichloro-phenyl)-6′-(3,5-dimethyl-phenyl)- | 2.12 | 413 | A |
| | | 3,4,5,6-tetrahydro-2H-[1,2′]bipyrazinyl | | | |
| 359 | 390.55 | 4-(3,4-Dimethyl-phenyl)-6′-(4-methylsulfanyl- | 1.59 | 391 | B |
| | | phenyl)-3,4,5,6-tetrahydro-2H-[1,2′]bipyrazinyl | | | |
| 360 | 375.47 | 2-(3,4-Dimethoxy-phenyl)-6-(4-Phenyl-piperidin- | 1.62 | 376 | B |
| | | 1-yl)-pyrazine | | | |
| 361 | 361.51 | 2-(4-Methylsulfanyl-phenyl)-6-(4-phenyl- | 1.87 | 362 | B |
| | | piperidin-1-yl)-pyrazine | | | |
| 362 | 357.46 | 1-{4-[6-(4-Phenyl-piperidin-1-yl)-pyrazin-2-yl]- | 1.77 | 358 | B |
| | | phenyl}-ethanone | | | |
| 363 | 364.43 | 6′-(4-Fluoro-phenyl)-4-(4-methoxy-phenyl)- | 1.37 | 365 | B |
| | | 3,4,5,6-tetrahydro-2H-[1,2′]bipyrazinyl | | | |
| 364 | 389.46 | 4-[4-(3-Methoxy-phenyl)-3,4,5,6-tetrahydro-2H- | 1.24 | 390 | B |
| | | [1,2′]bipyrazinyl-6′-yl]-benzamide | | | |
| 365 | 346.44 | 4-(3-Methoxy-phenyl)-6′-phenyl-3,4,5,6- | 1.54 | 347 | A |
| | | tetrahydro-2H-[1,2′]bipyrazinyl | | | |
| 366 | 376.46 | 4,6′-Bis-(3-methoxy-phenyl)-3,4,5,6-tetrahydro- | 1.56 | 377 | B |
| | | 2H-[1,2′]bipyrazinyl | | | |
| 367 | 364.43 | 6′-(4-Fluoro-phenyl)-4-(3-methoxy-phenyl)- | 1.58 | 365 | B |
| | | 3,4,5,6-tetrahydro-2H-[1,2′]bipyrazinyl | | | |
| 368 | 388.47 | 1-{3-[4-(3-Methoxy-phenyl)-3,4,5,6-tetrahydro- | 1.53 | 389 | B |
| | | 2H-[1,2′]bipyrazinyl-6′-yl]-phenyl}-ethanone | | | |
| 369 | 392.53 | 4-(3-Methoxy-phenyl)-6′-(4-methylsulfanyl- | 1.70 | 393 | B |
| | | phenyl)-3,4,5,6-tetrahydro-2H-[1,2′]bipyrazinyl | | | |
| 370 | 403.29 | 4-(3,4-Dichloro-phenyl)-6′-(4-fluoro-phenyl) | 2.03 | 403 | A |
| | | 3,4,5,6-tetrahydro-2H-[1,2′]bipyrazinyl | | | |
| 371 | 445.35 | 4-(3,4-Dichloro-phenyl)-6′-(3,4-dimethoxy- | 1.83 | 445 | A |
| | | phenyl)-3,4,5,6-tetrahydro-2H-[1,2′]bipyrazinyl | | | |
| 372 | 431.39 | 4-(3,4-Dichloro-phenyl)-6′-(4-methylsulfanyl- | 2.08 | 431 | A |
| | | phenyl)-3,4,5,6-tetrahydro-2H-[1,2′]bipyrazinyl | | | |
| 373 | 401.54 | 6′-Benzo[b]thiophen-2-yl-3′,5′-dimethyl-4-pyridin- | 1.33 | 402 | A |
| | | 4-yl-3,4,5,6-tetrahydro-2H-[1,2′]bipyrazinyl | | | |

| | |
|---|---|
| 374 | [6-(3-Amino-phenyl)-pyrazin-2-yl]-(3,4,5-trimethoxy-phenyl)-amine |
| 375 | N-{3-[6-(3,4,5-Trimethoxy-phenylamino)-pyrazin-2-yl]-phenyl}-acetamide |
| 376 | [6-(4-Isopropyl-phenyl)-pyrazin-2-yl]-(3,4,5-trimethoxy-phenyl)-amine |
| 377 | [6-(4-Methoxy-phenyl)-pyrazin-2-yl]-(2,2,3,3-tetrafluoro-2,3-dihydro- |
| | benzo[1,4]dioxin-6-yl)-amine |
| 378 | 3-[6-(2,2,3,3-Tetrafluoro-2,3-dihydro-benzo[1,4]dioxin-6-ylamino)-pyrazin-2-yl]- |
| | phenol |
| 379 | [6-(3-Amino-phenyl)-pyrazin-2-yl]-(3-chloro-phenyl)-amine |
| 380 | [6-(3-Amino-phenyl)-pyrazin-2-yl]-(4-chloro-phenyl)-amine |
| 381 | 3-[6-(4-Chloro-phenylamino)-pyrazin-2-yl]-N-(2-dimethylamino-ethyl)-benzamide |
| 382 | (3-Chloro-4-fluoro-phenyl)-[6-(3,4,5-trimethoxy-phenyl)-pyrazin-2-yl]-amine |
| 383 | 3-[6-(3-Chloro-4-fluoro-phenylamino)-pyrazin-2-yl]-N-(2-dimethylamino-ethyl)- |
| | benzamide |
| 384 | [6-(4-Methoxy-phenyl)-pyrazin-2-yl]-(3-trifluoromethyl-phenyl)-amine |
| 385 | (3-Chloro-phenyl)-[6-(3-chloro-phenyl)-pyrazin-2-yl]-amine |
| 386 | 4-(6-Phenylamino-pyrazin-2-yl)-phenol |
| 387 | N-{3-[6-(4-Chloro-phenylamino)-pyrazin-2-yl]-phenyl}-acetamide |
| 388 | 3-[6-(4-Methoxy-phenyl)-pyrazin-2-ylamino]-benzonitrile |
| 389 | 3-[6-(4-Amino-phenyl)-pyrazin-2-ylamino]-benzonitrile |
| 390 | [6-(3-Dimethylamino-phenyl)-pyrazin-2-yl]-(3,4,5-trimethoxy-phenyl)-amine |
| 391 | 3-[6-(3-Dimethylamino-phenyl)-pyrazin-2-ylamino]-benzonitrile |
| 392 | [6-(4-Ethoxy-phenyl)-pyrazin-2-yl]-(3,4,5-trimethoxy-phenyl)-amine |
| 393 | [6-(4-Amino-phenyl)-pyrazin-2-yl]-(3-trifluoromethyl-phenyl)-amine |
| 394 | (4-Chloro-phenyl)-[6-(3-dimethylamino-phenyl)-pyrazin-2-yl]-amine |
| 395 | [6-(3-Dimethylamino-phenyl)-pyrazin-2-yl]-phenyl-amine |
| 396 | 4-[6-(3,4,5-Trimethoxy-phenylamino)-pyrazin-2-yl]-phenol |
| 397 | N-{4-[6-(4-Chloro-phenylamino)-pyrazin-2-yl]-phenyl}-acetamide |
| 398 | [6-(4-Amino-phenyl)-pyrazin-2-yl]-(3,4,5-trimethoxy-phenyl)-amine |
| 399 | N-{4-[6-(3-Chloro-4-fluoro-phenylamino)-pyrazin-2-yl]-phenyl}-acetamide |
| 400 | [3-(6-Phenylamino-pyrazin-2-yl)-phenyl]-methanol |
| 401 | N-[4-(6-Phenylamino-pyrazin-2-yl)-phenyl]-acetamide |
| 402 | (6-Naphthalen-2-yl-pyrazin-2-yl)-(3,4,5-trimethoxy-phenyl)-amine |
| 403 | N-{3-[6-(2,2,3,3-Tetrafluoro-2,3-dihydro-benzo[1,4]dioxin-6-ylamino)-pyrazin-2-yl]- |
| | phenyl}-acetamide |
| 404 | 3-[6-(3-Chloro-phenylamino)-pyrazin-2-yl]-N-(2-dimethylamino-ethyl)-benzamide |
| 405 | N-{3-[6-(3-Chloro-4-fluoro-phenylamino)-pyrazin-2-yl]-phenyl}-acetamide |
| 406 | 4-(6-Phenylamino-pyrazin-2-yl)-benzamide |
| 407 | [6-(2,4-Difluoro-phenyl)-pyrazin-2-yl]-(3,4,5-trimethoxy-phenyl)-amine |
| 408 | [6-(4-Amino-phenyl)-pyrazin-2-yl]-(3-chloro-phenyl)-amine |
| 409 | N-{5-[6-(3-Dimethylamino-phenyl)-pyrazin-2-ylamino]-2-methyl-phenyl}- |
| | methanesulfonamide |
| 410 | N-{5-[6-(2,4-Difluoro-phenyl)-pyrazin-2-ylamino]-2-methyl-phenyl}- |
| | methanesulfonamide |
| 411 | [6-(4-Ethoxy-phenyl)-pyrazin-2-yl]-(3-trifluoromethyl-phenyl)-amine |
| 412 | 4-[6-(3-Chloro-phenylamino)-pyrazin-2-yl]-benzamide |
| 413 | 4-[6-(4-Amino-phenyl)-pyrazin-2-ylamino]-benzonitrile |
| 414 | 4-[6-(3,4,5-Trimethoxy-phenylamino)-pyrazin-2-yl]-benzamide |
| 415 | N-{3-[6-(3-Chloro-phenylamino)-pyrazin-2-yl]-phenyl}-acetamide |
| 416 | N-[3-(6-Phenylamino-pyrazin-2-yl)-phenyl]-acetamide |
| 417 | 3-[6-(3-Chloro-4-fluoro-phenylamino)-pyrazin-2-yl]-phenol |
| 418 | N-{3-[6-(3-Trifluoromethyl-phenylamino)-pyrazin-2-yl]-phenyl}-acetamide |
| 419 | (4-Chloro-phenyl)-[6-(3,4,5-trimethoxy-phenyl)-pyrazin-2-yl]-amine |
| 420 | {2-[6-(4-Chloro-phenylamino)-pyrazin-2-yl]-phenyl}-methanol |
| 421 | 3-[6-(3-Amino-phenyl)-pyrazin-2-ylamino]-benzonitrile |
| 422 | 3-[6-(5-Isopropyl-2-methoxy-phenyl)-pyrazin-2-ylamino]-benzonitrile |
| 423 | 3-[6-(3,4,5-Trimethoxy-phenylamino)-pyrazin-2-yl]-phenol |
| 424 | N-[2-Methyl-5-(6-naphthalen-2-yl-pyrazin-2-ylamino)-phenyl]-methanesulfonamide |
| 425 | 5-(3-Amino-phenyl)-N*3*-(4-methoxy-phenyl)-pyrazine-2,3-diamine |
| 426 | N*3*-(1H-Indol-5-yl)-5-(4-morpholin-4-yl-phenyl)-pyrazine-2,3-diamine |
| 427 | 5-(3-Amino-phenyl)-N*3*-(1H-indol-5-yl)-pyrazine-2,3-diamine |
| 428 | 4-[5-Amino-6-(1H-indol-5-ylamino)-pyrazin-2-yl]-phenol |
| 429 | 5-(3-Dimethylamino-phenyl)-N*3*-(1H-indol-5-yl)-pyrazine-2,3-diamine |
| 430 | N*3*-(1H-Indol-5-yl)-5-(4-methanesulfonyl-phenyl)-pyrazine-2,3-diamine |
| 431 | N*3*-(1H-Indol-5-yl)-5-(5-isopropyl-2-methoxy-phenyl)-pyrazine-2,3-diamine |
| 432 | N*3*-(1H-Indol-5-yl)-5-(3-methoxy-phenyl)-pyrazine-2,3-diamine |
| 433 | N*3*-(1H-Indol-5-yl)-5-quinolin-5-yl-pyrazine-2,3-diamine |
| 434 | 3-[5-Amino-6-(4-methoxy-phenylamino)-pyrazin-2-yl]-benzoic acid |
| 435 | 5-(4-Methanesulfonyl-phenyl)-N*3*-(4-methoxy-phenyl)-pyrazine-2,3-diamine |
| 436 | (E)-3-{3-[5-Amino-6-(4-methoxy-phenylamino)-pyrazin-2-yl]-phenyl}-acrylic acid |
| 437 | 5-(3-Bromo-phenyl)-N*3*-(4-methoxy-phenyl)-pyrazine-2,3-diamine |
| 438 | 5-(5-Isopropyl-2-methoxy-phenyl)-N*3*-(4-methoxy-2-methyl-phenyl)-pyrazine-2,3- |
| | diamine |
| 439 | {3-[5-Amino-6-(4-methoxy-2-methyl-phenylamino)-pyrazin-2-yl]-phenyl}-methanol |
| 440 | 3-[5-Amino-6-(4-methoxy-2-methyl-phenylamino)-pyrazin-2-yl]-benzamide |
| 441 | [6-(3-Amino-phenyl)-pyrazin-2-yl]-(3,4,5-trimethoxy-phenyl)-amine |

*Compounds characterized according to HPLC-MS method II

Furthermore, it was found that the pyridinylamines of the present invention are kinase inhibitors, especially of tyrosine kinases.

Table II (cf. below) shows the half-maximal inhibition concentration (IC₅₀) values of representative compounds according to general formula (I). Table II shows inhibition rates greater than 50% of various kinases. The results exhibited in both tables prove that the compounds of the present invention are potent pharmaceutically active agents against various diseases that can be treated and/or prohibited by inhibition of the targets \textcircled{2}-\textcircled{8}.

TABLE II
Inhibitory effect of the compounds of the present invention on different
targets (+ = >50% & ++ = >75% inhibition at concentration 10 μM)

| \textcircled{1} | \textcircled{2} | \textcircled{3} | \textcircled{4} | \textcircled{5} | \textcircled{6} | \textcircled{7} | \textcircled{8} |
|---|---|---|---|---|---|---|---|
| Comp. 375 | | ++ | + | + | + | + | |
| Comp. 396 | | ++ | ++ | ++ | + | ++ | |
| Comp. 379, 386, 390, 392, 398, 402, | | ++ | | | | | |
| 407, 415, 429, 430, 432, 433, 435, | | | | | | | |
| 437, 439, 440 | | | | | | | |
| Comp. 7, 29, 374, 376, 380, 382, | | + | | | | | |
| 384, 385, 387, 388, 391, 394, 395, | | | | | | | |
| 399, 400, 405, 406, 408-410, 412, | | | | | | | |
| 414, 416-428, 431, 434, 436, 438, | | | | | | | |
| Comp. 377, 378, 397, 403, 413 | + | | | | | | |
| Comp. 381, 383, 389, 404 | + | + | | | | | |
| Comp. 11 | ++ | + | | | | | |
| Comp. 22 | | | | | | | ++ |
| Comp. 401, 411 | | | | | | | + |

\textcircled{1} Compound Number
\textcircled{2} Target CDK9
\textcircled{3} Target RICK
\textcircled{4} Target c-Kit
\textcircled{5} Target p56Lck
\textcircled{6} Target EGFR
\textcircled{7} Target PDGFRbeta
\textcircled{8} Target SRPK1

Protein tyrosine kinases form a large family of structurally related enzymes that control a variety of different cell processes including proliferation, differentiation, apoptosis, motility, transcription, translation and other signaling processes by adding phosphate groups to target proteins (Hardie, G. and Hanks, S. (1995) The Protein Kinase Facts Book, I and II, Academic Press, San Diego, Calif.). The protein kinase family can conveniently be classified into two classes with regard to substrate specificity: protein tyrosine kinases (PTKs) phosphorylate their substrates on tyrosine residues, whereas serine/threonine kinases (STKs) phosphorylate proteins on serine or threonine residues.

PTKs can be further subdivided into receptor tyrosine kinases (RTKs) and intracellular tyrosine kinases. Upon binding of a ligand like a growth factor or hormone, RTKs are activated and, in turn, affect numerous cellular responses such as cell division (proliferation), cell differentiation, cell growth, expression of metabolic enzymes, effects to the extracellular microenvironment, etc. An example of a RTKs are EGFR (epithelial growth factor receptor), PDGFR, c-Kit and Insulin Receptor (InsR). Many of these RTKs have been implicated in cell proliferation disorders like cancer, and the EGFR inhibitors Iressa (Gefitinib) and Tarceva have been approved for treatment non-small cell lung cancer. (Nat Rev Drug Discov. 2003 April; 2(4):296-313; Nature Reviews Drug Discovery 3, 1001-1010 (2004); Nature Reviews Drug Discovery 3, 993-994 (2004).

Intracellular tyrosine kinases do not contain extracellular and transmembrane domains. One example of this group is the Src family of intracellular tyrosine kinases. These kinases are implicated in cancer, immune system dysfunction and bone remodeling diseases (For general reviews, see Thomas and Brugge, Annu. Rev. Cell Dev. Biol. (1997) 13, 513; Lawrence and Niu, Pharmacol. Ther. [(1998) 77, 81; Tatosyan and Mizenina, Biochemistry (Moscow) (2000) 65, 49; Boschelli et al., Drugs of the Future 2000, 25(7), 717, (2000)].

Members of the Src family include the following eight kinases in mammals: Src, Fyn, Yes, Fgr, Lyn, Hck, Lck, and Blk. Based on published studies, Src kinases are considered as potential therapeutic targets for various human diseases. The function of Lck as a positive activator of T-cell signaling suggests that Lck inhibitors may be useful for treating autoimmune disease such as rheumatoid arthritis (Molina et al., Nature, 357, 161 (1992)). Inhibition of these kinase mediators may therefore be useful for treating inflammation (Boschelli et al., Drugs of the Future 2000, 25(7), 717, (2000)).

An example for a STK family kinase is RICK (RIP2, Cardiak, CARD3). RICK belongs to the RIP family of protein kinases, including the kinases RICK, RIP, Rip3 and RIP4, which have been implemented in NF-kB activation. RICK is central part of the innate and adaptive immune response and involved in host response to intracellular infections as well as in inflammatory processes (Eickhoff et al. JBC March 2003; Current Biology, 8, p. 885-8; Nature 416, p. 194-9; Nature 416, p. 190-3). Inhibition of RICK has been described to modulate the innate and adaptive immune response (WO03059285). Inhibitors of RICK and RIP kinase activity have been described to block human Cytomegalovirus replication (US20030082519). The inventive compounds are explicitly suitable as RICK inhibitors.

Glycogen synthase kinase-3 (GSK-3) is a serine/threonine protein kinase, comprised of alpha and beta isoforms, that has been linked to various diseases including diabetes, Alzheimer's disease, CNS disorders such as manic depressive disorder and neurodegenerative diseases, and cardiomyocyte hypertrophy [see, e.g., WO 99/65897; WO 00/38675; Kaytor and Orr, Curr. Opin. Neurobiol., 12, 275-8 (2000); Haq et al., J. Cell Biol., 151, 117-30 (2000); Eldar-Finkelman, Trends Mol. Med., 8, 126-32 (2002)].

The Casein kinase I (CKI) gene family is another subfamily of serine/threonine protein kinases. This continuously expanding group of kinases have been implicated in the regulation of numerous cytoplasmic and nuclear processes, including cell metabolism and DNA replication and repair. CKI enzymes are present in the membranes, nucleus, cytoplasm and cytoskeleton of eukaryotic cells, and on the mitotic spindles of mammalian cells (Fish, K. J. et al. (1995) J. Biol. Chem. 270:14875-14883). Therefore the CKI enzyme is a target for pharmaceutical compounds influencing circadian rhythms, jet-lag and sleep, in addition to other physiologic and metabolic processes under circadian regulation (Lowrey, P. L. et al. (2000) Science 288:483-491).

Among the kinases, the cyclin-dependent kinases (CDKs) play a major role in the control of the cell cycle. To date, nine kinase subunits (cdk 1-9) have been identified along with several regulatory subunits (cyclins A-H) (A. M. Senderowicz and E. A. Sausville Journal of the National Cancer Institute (2000), 92 (5), 376-387; and S. Mani; C. Wang; K. Wu; R. Francis; R. Pestell' Exp. Opin. Invest. Drugs (2000) 9 (8), 1849-1870). An increasing body of evidence has shown a link between tumour development and COK related malfunctions. CDKs play a role in the regulation of cellular proliferation. Therefore, CDK inhibitors could be useful in the treatment of cell proliferative disorders (Lancet Oncol. 2004 January; 5(1):27-36. Review, Oncogene. 2003 Sep. 29; 22(42):6609-20, Curr Opin Pharmacol. 2003 August; 3(4):362-70). Other indications include neurodegenerative disorders such as Alzheimers disease and amyotrophic lateral sclerosis, which have been linked to Cdk5 (J Mol. Neurosci. 2002 December; 19(3):267-73). Several host cell kinases, including CDK9, have been shown to be important for virus replication like human cytomegalovirus, herpes simplex virus, human immune deficiency virus and VCV varicella zoster virus (WO2004/043467; J. Virol. 2004 March; 78(5):2517-29).

The human cytomegalovirus(HCMV)-encoded protein kinase pUL97 is belonging to a group of homologous protein kinase C (PKC)-like protein kinases with serine/threonine-specificity. Several studies have shown that pUL97 is particularly important for efficient replication (Marschall et al., 2001; Michel et al., 1996; Prichard et al., 1999; Wolf et al., 2001). Inhibitors of pUL97 should therefore be useful for treatment of HCMV associated diseases.

It has been clearly demonstrated that kinases play an important role in disease states associated with, but not limited to, disregulated cell signaling, inflammation, cancer, allergy/asthma, disease and conditions of the immune system, disease and conditions of the central nervous system, and angiogenesis. The development of selective protein kinase inhibitors that can block the disease pathologies and/or symptoms resulting from aberrant protein kinase activity has therefore generated much interest (Current review: Protein kinases—the major drug targets of the twenty-first century? Nat Rev Drug Discov. 2002 April; 1(4):309-15).

Attempts have been made to identify small organic molecules which inhibit protein kinases. For example, imidazoles, oxazoles and thiazoles (WO2004/005283), purines (2003/0199534) and bisindolyl-maleimids (WO9718809) have been described as kinase inhibitors. 3-(cycloalkano-heteroarylidenyl)-2-indolinone (U.S. Pat. No. 6,579,897), pyrimido-pyrimidines (US20040019210) and bis-monocylic, bicyclic and heterocyclic aryl compounds (WO 92/20642) have been described as specific PTK inhibitors. Some companies have begun to develop Inhibitors that specifically inhibit p38. For example, PCT publication WO02/14281 describes purines, PCT publication WO95/31451 describes pyrazoles and US 2004/0023992 describes pyrazolo-pyrimidine aniline compounds as p38 inhibitors. PCT publication WO 98/27098 also describes substituted nitrogen-containing heterocycles as p38 inhibitors.

The present invention also comprises pharmaceutically acceptable salts of the compounds according to the general formula (I), all stereoisomeric forms of the compounds according to the general formula (I) or prodrugs thereof. The following four compounds are excluded from the scope of this application by disclaimer: 4-(4-Pyridin-2-ylmethyl-3,4,5,6-tetrahydro-2H-[1,2′]bipyrazinyl-6′-yl)-phenol, 3-[4-(2-Cyano-phenyl)-3,4,5,6-tetrahydro-2H-[1,2′]bipyrazinyl-6′-yl]-N-(2-dimethyl-amino-ethyl)-benzamide, 2-Amino-3-{4-[4-(3,4-dimethoxy-phenyl)-3,4,5,6-tetrahydro-2H-[1,2′]bipyrazinyl-6′-yl]-phenyl}-propionic acid, 6′-(3-Chloro-4-fluoro-phenyl)-4-(2-pyrrolidin-1-yl-ethyl)-3,4,5,6-tetrahydro-2H-[1,2′]bipyrazinyl. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, d-o-tolyltartaric acid, tartronic acid, (o, m, p)-toluic acid, naphthylamine sulfonic acid, and other mineral or carboxylic acids well known to those skilled in the art. The salts are prepared by contacting the free base form with a sufficient amount of the desired acid to produce a salt in the conventional manner.

Depending upon the substituents of the inventive pyrazine compounds according to the general formula (I), one may be able to form salts with bases, too. Thus, for example, if there are carboxylic acid substituents in the molecule, salts may be formed with inorganic as well as organic bases such as, for example, NaOH, KOH, NH₄OH, tetraalkylammonium hydroxide, lysine or arginine and the like.

Salts may be prepared in a conventional manner using methods well known in the art, for example by treatment of a solution of the compound of the general formula (I) with a solution of an acid, selected out of the group mentioned above.

The present invention also includes prodrugs of the compounds according to the general formula (I). A prodrug is commonly described as an inactive or protected derivative of an active ingredient or a drug, which is converted to the active ingredient or drug in the body.

Some of the compounds of the present invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Certain compounds of the general formula (I) may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses. Where a compound according to the general formula (I) contains an alkene moiety, the alkene can be presented as a cis or trans isomer or a mixture thereof. When an isomeric form of a compound of the invention is provided substantially free of other isomers, it will preferably contain less than 5% w/w, more preferably less than 2% w/w and especially less than 1% w/w of the other isomers.

In a further preferred aspect of the present invention, the compounds according to the general formula (I) are used as new pharmaceutically active agents, particularly for the prophylaxis and/or treatment of infectious diseases, including opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke.

One particular aspect of the present invention relates to the use of the compounds disclosed herein for the prophylaxis and/or treatment of infectious diseases, including opportunistic diseases, pharmaceutical compositions comprising at least one compound according to the general formula (I) as active ingredients and a method for preventing and/or treating infectious diseases, including opportunistic diseases, in a mammal, including a human.

### Infectious Diseases Including Opportunistic Infections

The term infectious diseases comprises infections caused by viruses, bacteria, prions, fungi, and/or parasites.

Examples of infective diseases are AIDS, Alveolar Hydatid Disease (AHD, Echinococcosis), Amoebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Dipylidium Infection (Dog and Cat Flea Tapeworm), Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Fungal Dermatitis, Gastroenteritis, Group A streptococcal Disease, Group B streptococcal Disease, Hansen's Disease (Leprosy), Hantavirus Pulmonary Syndrome, Head Lice Infestation (Pediculosis), Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever.

### Virus Infections

Especially, virally induced infectious diseases, including opportunistic diseases are addressed. In a preferred embodiment of this aspect, the virally induced infectious diseases, including opportunistic diseases, are caused by retroviruses, human endogenous retroviruses (HERVs), hepadnaviruses, herpesviruses, flaviviridae, and/or adenoviruses. Preferably, the retroviruses are selected from lentiviruses or oncoretroviruses, wherein the lentivirus is preferably selected from the group comprising: HIV-1, HIV-2, and the oncoretrovirus is preferably selected from HTLV-I, HTLV-II or bovine leukemia virus (BLV),

The hepadnavirus is preferably selected from HBV, and the flaviviridae is selected West nile or Yellow Fever.

The herpes virusses are selected from human herpes viruses 1 to 8, and different herpes viruses for various animal species as shown below in Table 2.

TABLE 2
Members of the herpes virus family

| Subfamily | Genus | Human | Animal |
|---|---|---|---|
| α-herpesvirus | simplex virus | human herpesvirus 1 | bovine herpesvirus 2 |
| | | (herpes simplex virus 1) | |
| | | human herpesvirus 2 | cercopithecine herpesvirus |
| | | (herpes simplex virus 2) | 1, (herpes B virus) |
| | | | pseudorabiesvirus |
| | varicella virus | human herpesvirus 3 | bovine herpesvirus 1 |
| | | (Varicella Zoster virus) | equine-abortion virus |
| β-herpesvirus | cytomegalovirus | human herpesvirus 5 | |
| | | (HCMV) | |
| | muromegalovirus | | murine herpesvirus 1 |
| | Roseolovirus | human herpesvirus 6, | aotine herpesvirus 1, 3 |
| | | human herpesvirus 7 | |
| γ-herpesvirus | lymphocrytovirus | human herpesvirus 4 | cercopithecine herpesvirus 2 |
| | | (Epstein-Barr virus) | pongine herpesvirus 1 |
| | Rhadinovirus | human herpesvirus 8 | ateline herpesvirus 2 |
| | | | saimirine herpesvirus 1 |

Within the present invention herpes viruses are preferably selected from the group comprising:

α-herpesviruses (Simplexvirus, Varicellavirus), β-herpesviruses (Cytomegalovirus also known as human herpesvirus 5, or γ-herpesviruses (Lymphocryptovirus, Rhadinovirus). Examples for α-herpesviruses are Herpes simplex virus type I (human herpesvirus 1), Herpes simplex virus type 2 (human herpesvirus 2), Varicella Zoster virus (human herpesvirus 3). Examples for γ-herpesviruses are Epstein-Barr virus (human herpesvirus 4) or human herpesvirus type 8 (HHV8). Preferably, the herpesvirus is Herpes simplex virus type 1, or Varicella Zoster virus, or Epstein-Barr virus (EBV), or human cytomegalovirus (HCMV), or human herpesvirus 6, or human herpesvirus 7, or human herpesvirus type 8 (HHV8). More preferably, the herpesvirus represents the α-herpesviruses Herpes simplex virus type 1, or Varicella Zoster virus, or the γ-herpesviruses Epstein-Barr virus, or Human Herpes virus type 8 or most preferably the herpes virus represents the β-herpesvirus human herpesvirus 5. (HCMV).

In a further preferred embodiment of the present invention, the herpes virus is a drug resistant virus strain.

It is to be understood, that all the viruses mentioned above, also comprise drug resistant virus strains.

### Bacterial Infections

As described above, the compounds according to the general formula (I) are also useful for the preparation of a pharmaceutical composition for prophylaxis and/or treatment of bacterially induced infectious diseases, Including opportunistic diseases and opportunistic infections, wherein the bacterially induced infectious diseases, including opportunistic diseases, are selected from tuberculosis, leprosy or mycobacteria-induced meningitis. One advantage of the inventive compounds disclosed herein is there use against drug resistant bacteria strains.

### Prion Diseases

Another aspect of the present invention is directed to the use of at least one compound of the general formula (I) and/or pharmaceutically acceptable salts thereof for prophylaxis and/or treatment of prion diseases.

Prions are infectious agents, which do not have a nucleic acid genome. It seems that a protein alone is the infectious agent. A prion has been defined as “small proteinaceous infectious particle, which resists inactivation, by procedures that modify nucleic acids”. The discovery that proteins alone can transmit an infectious disease has come as a considerable surprise to the scientific community. Prion diseases are often called “transmissible spongiform encephalopathies”, because of the post mortem appearance of the brain with large vacuoles in the cortex and cerebellum. Probably most mammalian species develop these diseases. Prion diseases are a group of neurodegenerative disorders of humans and animals and the prion diseases can manifest as sporadic, genetic or infectious disorders. Examples for prion diseases acquired by exogenous infection are the Bovine spongiform encephalitis (BSE) of cattle and the new variant of Creutzfeld-Jakob disease (vCJD) caused by BSE as well as scrapie of animals. Examples of human prion diseases include kuru, sporadic Creutzfeldt-Jakob disease (sCJD), familial CJD (fCJD), iatrogenic CJD (iCJD), Gerstmann-Sträussler-Scheinker (GSS) disease, fatal familial insomnia (FFI), and especially the new variant CJD (nvCJD or vCJD).

The name “prion” is used to describe the causative agents, which underlie the transmissible spongiform encephalopathies. A prion is proposed to be a novel infectious particle that differs from viruses and viroids. It is composed solely of one unique protein that resists most inactivation procedures such as heat, radiation, and proteases. The latter characteristic has led to the term protease-resistant isoform of the prion protein. The protease-resistant isoform has been proposed to slowly catalyze the conversion of the normal prion protein into the abnormal form.

The term “isoform” in the context of prions means two proteins with exactly the same amino acid sequence, that are folded into molecules with dramatically different tertiary structures. The normal cellular isoform of the prion protein (PrP^{C}) has a high a-helix content, a low b-sheet content, and is sensitive to protease digestion. The abnormal, disease-causing isoform (PrP^{Sc}) has a lower a-helix content, a much higher b-sheet content, and is much more resistant to protease digestion.

As used herein the term “prion diseases” refers to transmissible spongiform encephalopathies. Examples for prion diseases comprise Scrapie (sheep, goat), TME (transmissible mink encephalopathy; mink), CWD (chronic wasting disease; muledeer, deer, elk), BSE (bovine spongiform encephalopathy; cows, cattles), CJD (Creutzfeld-Jacob Disease), vCJD, GSS (Gerstmann-Sträussler-Scheinker syndrome), FFI (Fatal familial Insomnia), Kuru, and Alpers Syndrome. Preferred are BSE, vCJD, and CJD.

Surprisingly, it was found that the compounds according to the present invention as well as pharmaceutically acceptable salts thereof are effective against infectious diseases, including opportunistic diseases, particularly herpes viral induced infections at pharmaceutically acceptable concentrations.

Furthermore, it was surprisingly found that the compounds of the present invention as well as pharmaceutically acceptable salts of these compounds are potent inhibitors of protein kinases, particularly of human and viral kinases. Especially, the viral kinase is a herpes viral kinase, preferably UL 97.

As used herein, a kinase “inhibitor” refers to any compound capable of downregulating, decreasing, suppressing or otherwise regulating the amount and/or activity of a kinase. Inhibition of these kinases can be achieved by any of a variety of mechanisms known in the art, including, but not limited to binding directly to the kinase polypeptide, denaturing or otherwise inactivating the kinase, or inhibiting the expression of the gene (e.g., transcription to mRNA, translation to a nascent polypeptide, and/or final polypeptide modifications to a mature protein), which encodes the kinase. Generally, kinase inhibitors may be proteins, polypeptides, nucleic acids, small molecules, or other chemical moieties.

As used herein the term “inhibiting” or “inhibition” refers to the ability of an inhibitor to downregulate, decrease, reduce, suppress, inactivate, or inhibit at least partially the activity of an enzyme, or the expression of an enzyme and the virus replication.

As used herein, a “pharmaceutically effective amount” of a kinase inhibitor is an amount effective to achieve the desired physiological result, either in cells treated in vitro or in a subject treated in vivo. Specifically, a pharmaceutically effective amount is an amount sufficient to inhibit, for some period of time, one or more of the clinically defined pathological processes associated with the viral infection. The effective amount may vary depending on the specific kinase inhibitor selected, and is also dependent on a variety of factors and conditions related to the subject to be treated and the severity of the infection. For example, if the inhibitor is to be administered in vivo, factors such as the age, weight and health of the patient as well as dose response curves and toxicity data obtained in preclinical animal work would be among those considered. If the inhibitor is to be contacted with the cells in vitro, one would also design a variety of pre-clinical in vitro studies to assess such parameters as uptake, half-life, dose, toxicity, etc. The determination of a pharmaceutically effective amount for a given agent is well within the ability of those skilled in the art.

### Immunological Diseases

Another aspect of the present invention is directed to the use of at least one compound of the general formula (I) and/or pharmaceutically acceptable salts thereof for prophylaxis and/or treatment of immunological diseases, neuroimmunological diseases, and autoimmune diseases.

Immunological diseases are, for instance, asthma and diabetes, rheumatic and autoimmune diseases, AIDS, rejection of transplanted organs and tissues (cf. below), rhinitis, chronic obstructive pulmonary diseases, osteoporisis, ulcerative colitis, sinusitis, lupus erythematosus, recurrent infections, atopic dermatitis/eczema and occupational allergies, food allergies, drug allergies, severe anaphylactic reactions, anaphylaxis, and other manifestations of allergic disease, as well as uncommon problems such as primary immunodeficiencies, including antibody deficiency states, cell mediated immunodeficiencies (e.g., severe combined immunodeficiency, DiGeorge syndrome, Hyper-IgE syndrome, Wiskott-Aldrich syndrome, ataxia-telangiectasia), immune mediated cancers, and white cell defects.

In autoimmune diseases, such as systemic lupus erythematosus, rheumatoid arthritis (RA), multiple sclerosis (MS), immune-mediated or type 1 diabetes mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, and Hashimoto's disease, dermatomyositis, goodpastture syndrome, myasthenia gravis pseudoparalytica, ophtalmia sympatica, phakogene uveitis, chronical aggressive hepatitis, primary billiary cirrhosis, autoimunehemolytic anemy, Werlof disease, specific cells uncontrollably attack the body's own tissues and organs (autoimmunity), producing inflammatory reactions and other serious symptoms and diseases.

Hashimoto's thyroiditis is one of the most common autoimmune diseases. “Autoimmune disease” refers to a category of more than 80 chronic illnesses, each very different in nature, that can affect everything from the endocrine glands (like the thyroid) to organs like the kidneys, as well as to the digestive system.

There are many different autoimmune diseases, and they can each affect the body in different ways. For example, the autoimmune reaction is directed against the brain in multiple sclerosis and the gut in Crohn's disease. In other autoimmune diseases such as systemic lupus erythematosus (lupus), affected tissues and organs may vary among individuals with the same disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus.

### Bipolar and Clinical Disorders

Another aspect of the present invention is directed to the use of at least one compound of the general formula (I) and/or pharmaceutically acceptable salts thereof for prophylaxis and/or treatment of bipolar and clinical disorders.

The term “bipolar and clinical disorders” shall refer to adjustment disorders, anxiety disorders, delirium, dementia, amnestic and other cognitive disorders, disorders usually first diagnosed in infancy, childhood, or adolescence, dissociative disorders, eating disorders, factitious disorders, impulse-control disorders, mental disorders due to a general medical condition, mood disorders, other conditions that may be a focus of clinical attention, personality disorders, schizophrenia and other psychotic disorders, sleep disorders, somatoform disorders, substance-related disorders, generalized anxiety disorder, panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, ADHD, obsessive-compulsive disorder (OCD), manic depressive psychosis.

Examples for delirium, dementia, amnestic and other cognitive disorders are: delirium due to a general medical condition, substance intoxication delirium, substance withdrawal delirium, delirium due to multiple etiologies, Alzheimer's, Creutzfeldt-Jakob disease, head trauma, Huntington's disease, HIV disease, Parkinson's disease, Pick's disease, substance-induced persisting, vascular, dementia due to other general medical conditions, dementia due to multiple etiologies, amnestic disorder due to a general medical condition, substance-induced persisting amnestic disorder.

Examples for disorders usually first diagnosed in infancy, childhood, or adolescence are: mental retardation, motor skills disorders, developmental coordination disorder, communication disorders, Tourette's syndrome.

Examples for dissociative disorders are: dissociative amnesia, depersonalization disorder, dissociative fugue and dissociative identity disorder.

Examples for eating disorders are anorexia nervosa and bulimia nervosa.

Examples for mood disorders are: mood episodes, major depressive episode, hypomanic episode, manic episode, mixed episode, depressive disorders, dysthymic disorder, major depressive disorder, single episode, recurrent, bipolar disorders, bipolar I disorder, bipolar II disorder, cyclothymic disorder, mood disorder due to a general medical condition, substance-induced mood disorder.

Examples for schizophrenia and other psychotic disorders are: schizophreniform disorder, schizoaffective disorder, delusional disorder, brief psychotic disorder, shared psychotic disorder, psychotic disorder due to a general medical condition, delusions, hallucinations, substance-induced psychotic disorder.

Examples for sexual and gender identity disorders are: female sexual arousal disorder, orgasmic disorders, premature ejaculation, sexual pain disorders, dyspareunia, vaginismus, sexual dysfunction due to a general medical condition, female dyspareunia, female hypoactive sexual desire disorder, male erectile disorder.

### Cardiovascular Diseases

Preferred are adult congenital heart disease, aneurysms, angina, angina pectoris, arrhythmias, cardiovascular disease prevention, cardiomyopathies, congestive heart failure, myocardial infarction, pulmonary hypertension, hypertrophic growth, restenosis, stenosis, thrombosis and arteriosclerosis.

### Proliferative Disease

In yet another preferred embodiment, the cell proliferative disease is cancer, preferably a solid tumour including but not limited to small and non small cell lung cancer, breast cancer, prostate cancer, colon cancer, skin cancer, gastric cancer or brain tumour.

The proliferation disorders and cancers are preferably selected from the group comprising adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

Preferred are the following cancer types: bladder, breast, central nervous system, colon, gastric, lung, kidney, melanoma, head and neck, ovarian, cervix, glioblastoma, pancreas, prostate, stomach, skin testis, leukemia, Hodgkin's lymphoma, liver and renal cancer.

### Diabetes

In yet another preferred embodiment, said diabetes is selected from Type I diabetes or Type II diabetes.

### Inflammation

In yet another preferred embodiment, said inflammation is mediated preferably by the cytokines TNF-α, IL-1β, GM-CSF, IL-6 and/or IL-8.

As described above, the compounds according to general formula (I) are pharmaceutically active agents for prophylaxis and/or treatment of inflammatory diseases. Thus, these compounds are used for the manufacture of a pharmaceutical formulation for prophylaxis and/or treatment of inflammations and inflammatory diseases in mammals, including humans.

Inflammatory diseases can emanate from infectious and non-infectious inflammatory conditions which may result from infection by an invading organism or from irritative, traumatic, metabolic, allergic, autoimmune, or idiopathic causes as shown in the following list.

| I. | Acute infections | |
|---|---|---|
| | A. Viral | B. Bacterial |
| II. | Noninfectious causes | |
| III. | Chronic (granulomatous) diseases | |
| | A. Bacterial | B. Spirochetal |
| | C. Mycotic (Fungal) | D. Idiopathic |

| IV. | Allergic, immune, and idiopathic disorders | |
|---|---|---|
| | A. Hypersensitivity reactions | |
| | B. Immune and idiopathic disorders | |
| V. | Miscellaneous inflammatory conditions | |
| | A. Parasitic infections | |
| | B. Inhalation causes: | Acute (thermal) injury |
| | | Pollution and inhalant allergy |
| | | Carcinogens |
| | C. Radiation injury: | Radionecrosis |

Thus, the compounds disclosed herein can be used for prophylaxis and/or treatment of inflammations caused by invading organisms such as viruses, bacteria, prions, and parasites as well as for prophylaxis and/or treatment of inflammations caused by irritative, traumatic, metabolic, allergic, autoimmune, or idiopathic reasons.

Consequently, the disclosed compounds are useful for prophylaxis and/or treatment of inflammatory diseases which are initiated or caused by viruses, parasites, and bacteria which are connected to or involved in inflammations.

The following bacteria are known to cause inflammatory diseases: mycoplasma pulmonis (causes e.g. chronic lung diseases (CLD), murine chronic respiratory disease), ureaplasma urealyticum (causes pneumonia in newborns), mycoplasma pneumoniae and chlamydia pneumoniae (cause chronic asthma), C. pneumoniae (causes atherosclerosis, pharyngitis to pneumonia with empyema, human coronary heart disease), Helicobacter pylori (human coronary heart disease, stomach ulcers).

The following viruses are known to cause inflammatory diseases: herpesviruses especially cytomegalovirus (causes human coronary heart disease).

The compounds disclosed herein are useful for prophylaxis and/or treatment of inflammatory diseases caused and/or induced and/or initiated and/or enhanced by the afore-mentioned bacteria or viruses.

Furthermore, the compounds of formula (I) are useful for prophylaxis and/or treatment of inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, inflammatory diseases of the larynx.

Examples for inflammatory diseases of the central nervous system (CNS) are algal disorders, protothecosis, bacterial disorders, abscessation, bacterial meningitis, idiopathic inflammatory disorders, eosinophilic meningoencephalitis, feline polioencephalomyelitis, granulomatous meningoencephalomyelitis, meningitis, steroid responsive meningitis-arteritis, miscellaneous meningitis/meningoencephalitis, necrotizing encephalitis, pyogranulomatous meningoencephalomyelitis, shaker dog disease, mycotic diseases of the CNS, parasitic encephalomyelitis, prion protein induced diseases, protozoal encephalitis-encephalomyelitis, toxoplasmosis, neosporosis, sarcocystosis, encephalitozoonosis, trypanosomiasis, acanthamebiasis, babesiosis, leishmaniasis, rickettsial disorders, rocky mountain spotted fever, viral disorders, aujeszky's disease, borna disease, canine distemper encephalomyelitis, chronic relapsing encephalomyelitis, La Crosse virus encephalitis, parvovirus encephalitis, rabies, post-vaccinal rabies.

Examples for inflammatory rheumatic diseases are rheumatoid arthritis, scleroderma, lupus, polymyositis, dermatomyositis, psoriatic arthritis, ankylosing spondylitis, Reiters's syndrome, juvenile rheumatoid arthritis, bursitis, tendinitis (tendonitis), and fibromyositis.

Examples for inflammatory diseases of blood vessels are vasculitis, autoantibodies in vasculitis, microscopic polyangiitis, giant cell arteritis, Takayasu's arteritis, vasculitis of the central nervous system, thromboangiitis obliterans (Buerger's Disease), vasculitis secondary to bacterial, fungal, and parasitic infection, vasculitis and rheumatoid arthritis, vasculitis in systemic lupus erythematosus, vasculitis in the idiopathic inflammatory myopathies, relapsing polychondritis, systemic vasculitis in sarcoidosis, vasculitis and malignancy, and drug-induced vasculitis.

Examples for inflammatory diseases of the middle ear are acute suppurative otitis media, bullous myringitis, granular myringitis, and chronic suppurative otitis media, which can manifest as mucosal disease, cholesteatoma, or both.

Examples for inflammatory bowel diseases are ulcerative colitis, Crohn's disease.

Examples for inflammatory diseases of the skin are acute inflammatory dermatoses, urticaria (hives), spongiotic dermatitis, allergic contact dermatitis, irritant contact dermatitis, atopic dermatitis, erythemal multiforme (EM minor), Stevens-Johnson syndrome (SJS, EM major), toxic epidermal necrolysis (TEN), chronic inflammatory dermatoses, psoriasis, lichen planus, discoid lupus erythematosus, and acne vulgaris

Uveitis are inflammations located in and/or on the eye and may be associated with inflammation elsewhere in the body. In most circumstances, patients who have uveitis as part of a disease elsewhere in the body are aware of that illness. The majority of patients with uveitis do not have an apparent associated systemic illness. Causes of uveitis can be infectious causes, masquerade syndromes, suspected immune-mediated diseases, and/or syndromes confined primarily to the eye.

The following viruses are associated with inflammations: human immunodeficiency virus-I, herpes simplex virus, herpes zoster virus, and cytomegalovirus.

Bacterial or spirochetal caused, induced, initiated and/or enhanced inflammations are tuberculosis, leprosy, proprionobacterium, syphilis, Whipple's disease, leptospirosis, brucellosis, and lyme disease.

Parasitic (protozoan or helminthic) caused, induced, initiated and/or enhanced inflammations are toxoplasmosis, acanthameba, toxocariasis, cysticercosis, onchocerciasis.

Examples of inflammatory diseases caused, induced, initiated and/or enhanced by fungi are histoplasmosis, coccidioidomycosis, candidiasis, aspergillosis, sporotrichosis, blastomycosis, and cryptococcosis.

Masquerade syndromes are, for instance, leukemia, lymphoma, retinitis pigmentosa, and retinoblastoma.

Suspected immune-mediated diseases can be selected from the group comprising ankylosing spondylitis, Behcet's disease, Crohn's disease, drug or hypersensitivity reaction, interstitial nephritis, juvenile rheumatoid arthritis, Kawasaki's disease, multiple sclerosis, psoriatic arthritis, Reiter's syndrome, relapsing polychondritis, sarcoidosis, Sjogren's syndrome, systemic lupus erythematosus, ulcerative colitis, vasculitis, vitiligo, Vogt Koyanagi Harada syndrome.

Syndromes confined primarily to the eye are, for instance, acute multifocal placoid pigmentary epitheliopathy, acute retinal necrosis, birdshot choroidopathy, Fuch's heterochromic cyclitis, glaucomatocyclitic crisis, lens-induced uveitis, multifocal choroiditis, pars planitis, serpiginous choroiditis, sympathetic ophthalmia, and trauma.

Examples for inflammatory diseases of the larynx are gastroesophageal (laryngopharyngeal) reflux disease, pediatric laryngitis, acute laryngeal infections of adults, chronic (granulomatous) diseases, allergic, immune, and idiopathic disorders and miscellaneous inflammatory conditions.

Pediatric laryngitis is known as acute (viral or bacterial) infection such as laryngotracheitis (croup), supraglottitis (epiglottitis), diphtheria, and noninfectious causes are for example spasmodic croup and traumatic laryngitis.

Acute laryngeal infections of adults are, for instance, viral laryngitis, common upper respiratory infection, laryngotracheitis, herpes simplex, bacterial laryngitis, supraglottitis, laryngeal abscess, and gonorrhea.

Chronic (granulomatous) diseases can be selected from the group comprising bacterial diseases, tuberculosis, leprosy, scleroma, actinomycosis, tularemia, glanders, spirochetal (syphilis) diseases, mycotic (fungal) diseases, candidiasis, blastomycosis, histoplasmosis, coccidiomycosis, aspergillosis, idiopathic diseases, sarcoidosis, and Wegener's granulomatosis.

Allergic, immune, and idiopathic disorders are, for example, hypersensitivity reactions, angioedema, Stevens-Johnson syndrome, immune and idiopathic disorders, infections of the immunocompromised host, rheuatoid arthritis, systeic lupus erythematosus, cicatricial pemphigoid, relapsing polychondritis, Sjogren's syndrome, and amyloidosis.

Miscellaneous inflammatory conditions are, for instance, parasitic infections, trichinosis, leishmaniasis, schistosomiasis, syngamus laryngeus, inhalation laryngitis, acute (thermal) injury, pollution and inhalant allergy, carcinogens, radiation injury, radiation laryngitis, radionecrosis, vocal abuse, vocal-cord hemorrhage, muscle tension dysphonias, and contact ulcer and granuloma.

### Transplant Rejection

Transplant rejection is when a transplant recipient's immune system attacks a transplanted organ or tissue. No two people (except identical twins) have identical tissue antigens. Therefore, in the absence of immunosuppressive drugs, organ and tissue transplantation would almost always cause an immune response against the foreign tissue (rejection), which would result in destruction of the transplant. Though tissue typing ensures that the organ or tissue is as similar as possible to the tissues of the recipient, unless the donor is an identical twin, no match is perfect and the possibility of organ/tissue rejection remains.

The inventive compounds of general formula (I) are used as immunosuppressive drugs and/or anti-rejection drugs in order to prevent transplant rejection.

One example of transplant rejection is the graft-versus-host-disease (GVHD) that can occur following bone marrow transplant. The donor's immune cells in the transplanted marrow make antibodies against the host's (transplant patient's) tissues and attack the patient's vital organs. Transplant rejections (also known as graft rejection or tissue/organ rejection) may commonly occur when tissue or organs, which need blood supply, are transplanted. Said organs comprise especially inner organs such as heart, heart-lungs, lungs, liver, kidney, pancreas, spleen, skin, tissue, bone marrow, spinal marrow, hormone producing glands, gonads and gonadal glands.

### Neurodegenerative Diseases

Another aspect of the present invention is directed to the use of at least one compound of the general formula (I) and/or pharmaceutically acceptable salts thereof for prophylaxis and/or treatment of neurodegeneration and neurodegenerative disorders.

Among the hundreds of different neurodegenerative disorders, the attention has been given only to a handful, including Alzheimer disease, Parkinson disease, Huntington disease, and amyotrophic lateral sclerosis.

It is worth to mention that the same neurodegenerative process can affect different areas of the brain, making a given disease appear very different from a symptomatic standpoint.

Neurodegenerative disorders of the central nervous system (CNS) can be grouped into diseases of the cerebral cortex (Alzheimer disease), the basal ganglia (Parkinson disease), the brain-stem and cerebellum, or the spinal cord (amyotrophic lateral sclerosis).

Examples for neurodegeneration and neurodegenerative disorders are Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA).

In another aspect of the present invention, the compounds according to the general formula (I) as well as pharmaceutically acceptable salts thereof are used as an inhibitor for a protein kinase, preferably as an inhibitor for a cellular protein kinase. Table 1 shows a list with all currently known cellular protein kinases.

In a preferred embodiment of this aspect said cellular protein kinase is selected from the group consisting of:

Cyclin-dependent protein kinase (CDK), protein kinase C, c-Raf, Akt, CKI, IKKβ, MAP kinases/ERKs, MAP kinases/JNKs, EGF receptor, InsR, PDGF receptor, c-Met, p70S6K, ROCK, Rsk1, Src, Abl, p56Lck, c-kit, CaMk2β, CaMk2δ, CaMk2γ, CSK or GSK-3α and β. The cyclin-dependent protein kinase can be selected from the group comprising:
CDK1, CDK2, CDK3, CDK4, CDK5, CDK6, CDK7, CDK8, CDK9, CDK10, CDK11, CrkRS (Crk7, CDC2-related protein kinase 7), CDKL1 (cyclin-dependent kinase-like 1); KKIALRE, CDKL2 (cyclin-dependent kinase-like 2), KKIAMRE, CDKL3 (cyclin-dependent kinase-like 3), NKIAMRE, CDKL4, similar to cyclin-dependent kinase-like 1, CDC2L1 (cell division cycle 2-like 1), PITSLRE B, CDC2L1 (cell division cycle 2-like 1), PITSLRE A, CDC2L5 (cell division cycle 2-like 5), PCTK1 (PCTAIRE protein kinase 1), PCTK2 (PCTAIRE protein kinase 2), PCTK3 (PCTAIRE protein kinase 3) or PFTK1 (PFTAIRE protein kinase 1). Preferred are: CDK9, RICK, c-Kit, p56Lck, EGFR, PDGFRbeta, SRPK1, UL97.

In a further aspect of the present invention, a method for preventing and/or treating infectious diseases, including opportunistic diseases, in a mammal, especially in a human, is provided, which method comprises administering to the mammal an amount of at least one compound according to the general formula (I), effective to prevent and/or treat said infectious diseases, including opportunistic diseases. In a preferred embodiment of this method, the infectious diseases, including opportunistic diseases, are virally induced infectious diseases. The virally induced infectious diseases, including opportunistic diseases, are caused by retroviruses, hepadnaviruses, herpesviruses, flaviviridae, and/or adenoviruses. In a further preferred embodiment of this method, the retroviruses are selected from lentiviruses or oncoretroviruses, wherein the lentivirus is selected from the group comprising: HIV-1, HIV-2, FIV, BIV, SIVs, SHIV, CAEV, VMV or EIAV, preferably HIV-1 or HIV-2 and wherein the oncoretrovirus is selected from the group consisting of: HTLV-I, HTLV-II or BLV. In a further preferred embodiment of this method, the hepadnavirus is selected from HBV, GSHV or WHV, preferably HBV, the herpesivirus is selected from the group comprising: HSV I, HSV II, EBV, VZV, HCMV or HHV 8, preferably HCMV and the flaviviridae is selected from HCV, West nile or Yellow Fever.

In yet another aspect of the present invention, the compounds according to the general formula (I) are used for the preparation of a pharmaceutical composition for the prophylaxis and/or treatment of infectious diseases, including opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases, stroke and especially of infectious diseases caused by herpes viruses, particularly those herpes viruses mentioned above.

The present invention also provides a method for preventing and/or treating infectious diseases, including opportunistic diseases, especially infectious diseases caused by herpes viruses in a mammal, particularly in a human, which method comprises administering to the mammal an amount of at least one of the compounds of the present invention and/or pharmaceutically acceptable salts thereof effective to treat a herpes viral induced infection, such as herpes.

The compounds shown explicitly in Table 1 are preferred to be used within the methods or for the indications disclosed herein. Another aspect of the present invention is that at least one compound of the present invention used as an pharmaceutically active agent may be administered in combination with further therapeutic compounds selected from the group comprising of:

Ganciclovir, foscarnet, cidofovir, valganciclovir, ganciclovir implants, fomivirsen, penciclovir and valaciclovir.

Within said methods the compounds according to the general formula (I) and/or pharmaceutically acceptable salts thereof are administered in a dosage corresponding to an effective concentration in the range of 0.001-50 μM, preferably in the range of 0.002-10 μM, more preferably in the range of 0.003-1 μM.

According to a still further aspect, the present invention refers to pharmaceutical compositions comprising at least one compound according to the present invention as an active ingredient together with at least one pharmaceutically acceptable (i.e. non-toxic) carrier, excipient and/or diluent. The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, powders and deposits.

The pharmaceutically effective compounds of formula (I) and pharmaceutically acceptable salts and prodrugs thereof, may be administered in conventional dosage forms prepared by combining a compound of formula (I) (“active ingredient”) with standard pharmaceutical carriers or excipients according to conventional procedures well known in the art. These procedures may involve mixing, granulating and compressing or dissolving the ingredients as appropriate to the desired preparation.

According to a further aspect of the present invention there is provided a pharmaceutical composition comprising at least one compound of the general formula (I), or a pharmaceutically acceptable salt or prodrug thereof, together with one or more pharmaceutically acceptable carriers or excipients.

Furthermore, the present invention also includes pharmaceutical formulations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain at least one compound according to the present invention and/or a pharmaceutical acceptable salt thereof as active ingredient.

The pharmaceutical formulation of the present invention may be formulated for administration by any route, and include those in a form adapted for oral, topical or parenteral administration to mammals including humans.

Pharmaceutical formulations may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical formulations adapted for transdermal administration may be presented as discrete patches intended to remain in intimate contact with the epidermis of the recipient for a prolonged period of time.

Pharmaceutical formulations adapted for topical administration may be formulated as ointments, creams, suspensions, lotions, powders, solutions, pastes, gels, impregnated dressings, sprays, aerosols or oils and may contain appropriate conventional additives such as preservatives, solvents to assist drug penetration and emollients in ointments and creams.

For applications to the eye or other external tissues, for example the mouth and skin, the formulations are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administration to the eye include eye drops wherein the active ingredient is dissolved or suspended in a suitable carrier, especially an aqueous solvent. Pharmaceutical formulations adapted for topical administration in the mouth include lozenges, pastilles and mouth washes.

Pharmaceutical formulations adapted for rectal administration may be presented as suppositories or enemas. Pharmaceutical formulations adapted for nasal administration wherein the carrier is a solid include a coarse powder having a particle size for example in the range 20 to 500 microns. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists which may be generated by means of various types of metered dose pressurised aerosols, nebulizers or insufflators.

Pharmaceutical formulations adapted for vaginal administration may be presented as pessaries, tampons, creams, gels, pastes, foams or spray formulations.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations may also include other agents conventional in the art having regard to the type of formulation in question, for example those suitable for oral administration may include flavouring agents.

The pharmaceutical formulations according to the present invention are preferably adapted for oral administration.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1%-100% by weight, preferably from 10-80% by weight, of the active material, depending on the method of administration.

Pharmaceutical formulations may be presented in unit dose forms containing a predetermined amount of active ingredient per dose. Such a unit may contain for example 100 mg/kg to 1 mg/kg depending on the condition being treated, the route of administration and the age, weight and condition of the patient. Preferred unit dosage formulations are those containing a daily dose or sub-dose, as herein above recited, or an appropriate fraction thereof, of the active ingredient.

It will be recognized by one of skill in the art that the optimal quantity and spacing of individual dosages of a formula (I) compound will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of the compound of formula (I) given per day for a defined number of days, can be ascertained by those skilled in the art using conventional course of treatment determination tests.

Since the compounds of the general formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis).

The compounds according the general formula (I) can be prepared by art-recognized procedures from known or commercially available starting materials. If the starting materials are unavailable from a commercial source, their synthesis is described herein, or they can be prepared by procedures known in the art. The present invention also provides processes for preparing a compound of the general formula (I).

The general procedure for synthesizing compounds of the general formula (I) is illustrated in Scheme I:

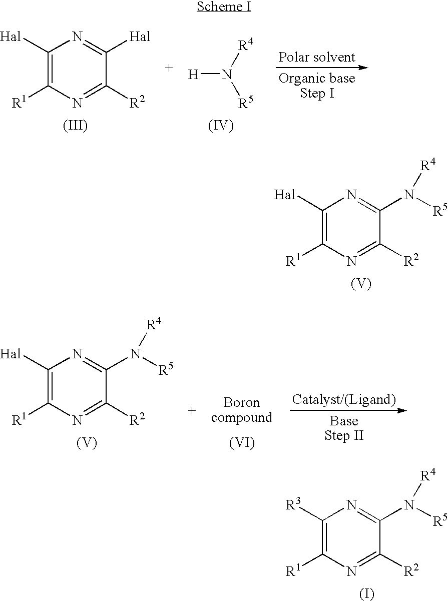

In a first reaction step (Step I) a 2,6 dihalogenated pyrazine derivative according to formula (III) is reacted with an amine compound of the formula (IV) to give a compound of the formula (V), wherein the reaction is carried out in the presence of a suitable polar solvent, such as ethanol, methanol or THF and in the presence of an organic base, for example triethylamine or diisopropylethylamine (Hünig's base). The reaction is carried out at a temperature required for the corresponding reaction, this means the temperature range varies from room temperature to reflux temperatures. Compounds of formulae (III) and (IV) may be available from commercial sources or may be prepared by methods well known to those skilled in the art. See for example (a) Karmas, G.; Spoerri, P. E. J. Am. Chem. Soc. 1952, 74, 1580. (b) Sato, N.; Matsumoto, K.; Takishima, M.; Mochizuki, K. J. Chem. Soc., Perkin Trans. 1 1997, 3167. (c) Cacchi, S.; Carangio, A.; Fabrizi, G.; Moro, L.; Pace, P. Synlett 1997, 12, 1400. (d) Morita, S.; Kitano, K.; Matsubara, J.; Ohtani, T.; Kawano, Y.; Otsubo, K.; Uchida, M. Tetrahedron 1998, 54, 4811. (e) Torisawa, Y.; Nishi, T., Minamikawa, J.; Bioorg. Med. Chem. Lett. 2000, 10, 2489.

In a next reaction step (Step II), a compound of the formula (V) is reacted with a boron compound (VI) via a Suzuki coupling in the presence of a catalyst or a catalyst/ligand system and a base in an organic solvent or a mixture of an organic solvent and water to give a compound of the general formula (I).

The boron compound (VI) may be selected from R³—B(OH)₂, R³—B(OiPr)₂, R³-9-BBN or

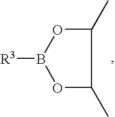

the catalyst or catalyst/ligand system may be selected from the group comprising:
Pd(PPh₃)₄, Pd(dppf)(OAc)₂, Pd(OAc)₂, Pd₂(dba)₃/P(t-Bu)₃, Pd(PCy₃)₂Cl₂ or Pd/C+PPh₃,
the base may be selected from the group comprising:
Cs₂CO₃, Na₂CO₃, K₂CO₃, Ba(OH)₂, K₃PO₄, TIOH, KF or NaOH
and the organic solvent or solvent/water mixture can be DME, DMF, THF, Dioxane, MeOH or benzene and the mixture of an organic solvent/water can be selected from the group comprising: DME/water, DMF/water or THF/water.

This reaction step can be carried out under conditions required for this reaction, for example the reaction is carried out under heating and/or stirring using a conventional hot plate stirrer or heating in a microwave reactor.

This reaction type is described in:

(a) Thompson, W. J.; Jones, J. H.; Lyle, P. A.; Thies, J. E.; J. Org. Chem. 1988, 53, 2052. (b) Miyaura, N.; Suzuki, A. Chem. Rev. 1995, 95, 2457. (c) Stanforth, S. P. Tetrahedron 1998, 54, 263. (d) Kotha, S.; Lahiri, K.; Kashinath, D. Tetrahedron 2002, 58, 9633. (e) Hassan, J.; Sévignon, M.; Gozzi, C.; Schulz, E.; Lemaire, M. Chem. Rev. 2002, 102, 1359.

According to Scheme (I)

Hal represents —Cl, —Br or —I, preferably —Cl,
R¹ and R² have the meaning as defined in claim 1, preferably R¹ and R² are independently selected from the group comprising: —H, linear or branched C₁-C₄ alkyl or NH₂,
R⁴ and R⁵ have the meaning as defined in claim 1, preferably R⁴ and R⁵ form a ring system according to the general formula (II)

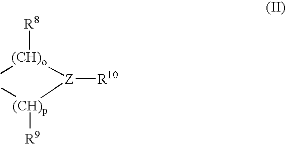

wherein o and p are independently selected to be an integer from 1 to 3,
Z is selected from CH or N,
each R⁸ and each R⁹ represent independently from each other —H, linear or branched C₁-C₆ alkyl or —(CH₂)ᵤ—OH, wherein u is selected to be an integer from 0 to 6 and if u is selected to be an integer from 2 to 6, at least one, preferably one or two hydrogen atoms bonded to the —(CH₂)ᵤ carbon chain are optionally substituted by —F, —Cl, —Br, —I, —OH, —NH₂, linear or branched C₁-C₆ alkyl or linear or branched C₁-C₆ alkoxy and
R¹⁰ is selected from the group comprising:
—H, linear or branched C₁-C₆ alkyl, C₃-C₈ cycloalkyl, which is optionally partially or fully substituted, aryl, which is optionally partially or fully substituted, heteroaryl which is optionally partially or fully substituted or heterocyclyl, which is optionally partially or fully substituted,
—C(O)—R¹¹ or —(CH₂)_{q}—R¹¹
wherein q is an integer from 0 to 6 and R¹¹ is selected from aryl, which is optionally partially or fully substituted, heteroaryl, which is optionally partially or fully substituted or heterocyclyl, which is optionally partially or fully substituted.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in for example Protective Groups in Organic Chemistry, T. W. Greene and P. G. M. Wuts, (Wiley-Interscience, New York, 2nd edition, 1991). Further details for the preparation of compounds of formula (I) are found in the examples.

The compounds of formula (I) may be prepared singly or as compound libraries comprising at least 2, for example 5 to 1,000 compounds, and more preferably 10 prepared by multiple parallel synthesis using either solution phase or solid phase chemistry, by procedures known to those skilled in the art.

Thus according to a further aspect of the invention there is provided a compound library comprising at least 2 compounds of formula (I) or pharmaceutically acceptable salts and prodrugs thereof.

### DESCRIPTION OF FIGURES

FIG. 1 shows the inhibition of HCMV replication (HCMV Replication assay) by using several pyrazine derivatives of the present invention at different concentrations (GCV=Ganciclovir).

FIG. 2 shows comparative control experiments with cells containing virus (first column), DMSO solution (second column), ganciclovir (third column), and cells without virus (Mock).

FIG. 3 shows representative examples of the inventive pyrazine compounds.

FIG. 4 shows the results of a pUL97 in-cell-activity assay of representative compounds of the present invention in comparison to the specific inhibitor NGICI-I which belongs to the compound class of indolocarbazoles.

### EXPERIMENTAL PART

### 1) Synthesis of Compounds:

Commercially available reagents and solvents (HPLC grade) were used without further purification.

Microwave irradiation was carried out using a CEM Discover focused microwave reactor.

For high-throughput reactions, sample transfer and filtration was carried out using Zinsser Lissy liquid handling robots. Analytical plates were prepared using a Beckman Biomek 2000 liquid handling robot.

Solvents were removed using a GeneVac Series I without heating or a Genevac Series II with VacRamp at 40° C.

Purification of compounds by column chromatography was carried out using a Biotage Horizon HPFC system. Purification of compounds by preparative HPLC was performed on Gilson systems using reverse phase ThermoHypersil-Keystone Hyperprep HS C18 columns (12 μm, 100×21.2 mm), gradient 20-100% B (A=water/0.1% TFA, B=acetonitrile/0.1% TFA) over 9.5 min, flow=30 ml/min, injection solvent 2:1 DMSO:acetonitrile (1.6 ml), UV detection at 215 nm.

¹H NMR spectra were recorded on a Bruker 400 MHz AV spectrometer in deuterated solvents. Chemical shifts (δ) are in parts per million and coupling constants are expressed in Hz. Thin-layer chromatography (TLC) analysis was performed with Kieselgel 60 F₂₅₄ (Merck) plates and visualized using UV light.

### Analytical HPLC-MS Method I:

Analytical HPLC-MS was performed on Agilent HP1100, Waters 600 or Waters 1525 LC systems using reverse phase Hypersil BDS C18 columns (5 μm, 2.1×50 mm), gradient 0-95% B (A=water/0.1% TFA, B=acetonitrile/0.1% TFA) over 2.10 min, flow=1.0 ml/min. UV spectra were recorded at 215 nm using a Gilson G1315A Diode Array Detector, G1214A single wavelength UV detector, Waters 2487 dual wavelength UV detector, Waters 2488 dual wavelength UV detector, or Waters 2996 diode array UV detector. Mass spectra were obtained over the range m/z 150 to 850 at a sampling rate of 2 scans per second or 1 scan per 1.2 seconds using Micromass LCT with Z-spray interface or Micromass LCT with Z-spray or MUX interface. Data were integrated and reported using OpenLynx and OpenLynx Browser software.

### Analytical HPLC-MS Method II:

Analytical HPLC/MS was performed on a Waters Alliance 2795 HT HPLC coupled to a Waters ZQ2000 single-quadrupole mass spectrometer. UV spectra were recorded using a Waters 2996 photodiode array detector. Chromatography was performed using the parameters cited below:

| Solvents: | Acetonitrile (Lichrosolv Merck) | | |
|---|---|---|---|
| | Water (Lichrosolv Merck) with 1 mM ammonium | | |
| | acetate pH 6.8 | | |
| Column: | Zorbax Bonus RP 3.5 μm, 4.6 × 75 mm. | | |
| Flow Rate: | 0.8 ml/min | | |
| Injection volume: | 20 uL | | |
| Gradient: | A: Water/NH₄OAc B: MeCN | | |
| | Time | A % | B % |
| | 0.00 | 100 | 0 |
| | 1.50 | 100 | 0 |
| | 8.50 | 15 | 85 |
| | 8.60 | 2 | 98 |
| | 11.60 | 2 | 98 |
| | 11.70 | 100 | 0 |
| | 13.50 | 100 | 0 |

UV spectra were recorded from 210 to 700 nm with a sampling rate of 1.2 spectra/second. Mass spectra were obtained using positive and negative electrospray ionization over the range m/z 110 to 600. The scan rate was 1 scan (m/z 150 to 600) per second.

### Examples

The compounds of this invention may be prepared according to one of the examples shown below, most preferably according to Scheme I, illustrated in Example 13. Compounds according to this invention are incorporated in Table 1.

### Example 1

### Stage 1: 6′-Chloro-4-pyridin-4-yl-3,4,5,6-tetrahydro-2H-[1,2′]bipyrazinyl

2,6-Dichloropyrazine (1.20 g, 8.0 mmol), 1-(4-pyridyl)piperazine (1.43 g, 8.8 mmol) and triethylamine (1.7 ml, 12.0 mmol) were dissolved in ethanol (50 ml). The reaction mixture was heated at reflux for 16 h, allowed to reach ambient temperature and dried under reduced pressure. The crude material was dissolved in chloroform, washed with saturated aqueous sodium bicarbonate, dried over MgSO₄ and the solvent removed in vacuo. The residue was washed with ethyl acetate and dried under reduced pressure to give the required product as a yellow powder.

Yield: 943 mg (43%)

Mass spectrum (ES-MS (+ve)) 276 [M+H]⁺, Retention time 0.94 min.

¹H-NMR (DMSO-d₆, 400 MHz): δ 8.31 (1H, s, Ar), 8.16 (2H, d, J=6.4 Hz, Ar), 7.87 (1H, s, Ar), 6.84 (2H, d, J=6.4 Hz, Ar), 3.71 (4H, m, 2×NCH₂), 3.46 (4H, m, 2×NCH₂).

### Stage 2: 4-(4-Pyridin-4-yl-3,4,5,6-tetrahydro-2H-[1,2′]bipyrazinyl-6′-yl)-benzamide (Comp. 21)

6′-Chloro-4-pyridin-4-yl-3,4,5,6-tetrahydro-2H-[1,2′]bipyrazinyl (100 mg, 0.36 mmol), 4-(aminocarbonylphenyl)boronic acid (65 mg, 0.40 mmol), cesium carbonate (234 mg, 0.72 mmol) and palladium tetrakistriphenylphosphine (8 mg, 0.007 mmol), were added to a microwave tube and diluted with 5 ml of a mixture of toluene:EtOH (4:1). The tube was sealed and placed in a CEM Discover microwave (power 150 W, temperature 120° C.) for 20 min and then cooled to ambient temperature. The reaction was repeated using the same amounts of materials and the same reaction conditions. The two crude reaction mixture were combined and filtered through a short pad of celite. The solvent was removed and the residue washed with diethyl ether and heptane. The product was purified by recrystallized from methanol.

Yield: 102 mg (39%)

Mass spectrum (ES-MS (+ve)) 361 [M+H]⁺, Retention time 0.79 min.

¹H-NMR (MeOH-d₄, 400 MHz): δ 8.32 (1H, s, Ar), 8.13 (1H, s, Ar), 8.07 (2H, d, J=8.6 Hz, Ar), 8.05 (2H, br d, Ar), 7.89, (2H, d, J=8.6 Hz, Ar), 6.80 (2H, d, J=6.49 Hz, Ar), 3.82 (4H, m, 2×NCH₂), 3.52 (4H, m, 2×NCH₂).

### Example 2

### Stage 1: 6′-Chloro-4-pyridin-4-yl-3,4,5,6-tetrahydro-2H-[1,2′]bipyrazinyl was Prepared as for Example 1 Stage 1

### Stage 2: 6′-Naphthalen-2-yl-4-pyridin-4-yl-3,4,5,6-tetrahydro-2H-[1,2-′]bipyrazinyl (Comp. 72)

6′-Chloro-4-pyridin-4-yl-3,4,5,6-tetrahydro-2H-[1,2′]bipyrazinyl (50 mg, 0.18 mmol), 2-naphthalene boronic acid (43 mg, 0.25 mmol), cesium carbonate (117 mg, 0.36 mmol) and palladium tetrakistriphenylphosphine (8 mg, 0.007 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the reaction mixture filtered through a short pad of celite. The solvent was removed under reduced pressure, the residue washed with diethyl ether, dissolved in chloroform, washed with saturated aqueous sodium carbonate and dried over MgSO₄. The solvent was removed in vacuo and the product was purified by prep-HPLC.

Yield: 7.8 mg (8%)

Mass spectrum (ES-MS (+ve)) 368 [M+H]⁺, Retention time 1.24 min.

### Example 3

### Stage 1: 6′-Chloro-4-pyridin-4-yl-3,4,5,6-tetrahydro-2H-[1,2′]bipyrazinyl was Prepared as for Example 1 Stage 1

### Stage 2 6′-Benzo[b]thiophen-2-yl-4-pyridin-4-yl-3,4,5,6-tetrahydro-2H-[1,2′]bipyrazinyl (Compound 69)

6′-Chloro-4-pyridin-4-yl-3,4,5,6-tetrahydro-2H-[1,2′]bipyrazinyl (50 mg, 0.18 mmol), thianaphthene-2-boronic acid (45 mg, 0.25 mmol), cesium carbonate (117 mg, 0.36 mg) and palladium tetrakistriphenylphosphine (8 mg, 0.007 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the reaction mixture filtered through a short pad of celite. The solvent was removed under reduced pressure, the residue washed with diethyl ether, dissolved in chloroform, washed with saturated aqueous sodium carbonate and dried over MgSO₄. The solvent was removed in vacuo and the product was purified by prep-HPLC.

Yield: 9.7 mg (14%)

Mass spectrum (ES-MS (+ve)) 374 [M+H]⁺, Retention time 1.30 min.

### Example 4

### Stage 1: 6′-Chloro-4-pyridin-4-yl-3,4,5,6-tetrahydro-2H-[1,2′]bipyrazinyl was Prepared as for Example 1 Stage 1

### Stage 2: 3-(4-Pyridin-4-yl-3,4,5,6-tetrahydro-2H-[1,2′]bipyrazinyl-6′-yl)-benzamide (Comp. 71)

6′-Chloro-4-pyridin-4-yl-3,4,5,6-tetrahydro-2H-[1,2′]bipyrazinyl (50 mg, 0.18 mmol), 3-(aminocarbonylphenyl)boronic acid (41 mg, 0.25 mmol), cesium carbonate (117 mg, 0.36 mmol) and palladium tetrakistriphenylphosphine (8 mg, 0.007 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the reaction mixture filtered through a short pad of celite. The solvent was removed under reduced pressure, the residue washed with diethyl ether, dissolved in chloroform, washed with saturated aqueous sodium carbonate and dried over MgSO₄. The solvent was removed in vacuo and the product was purified by recrystallization from methanol.

Yield: 5.8 mg (9%)

Mass spectrum (ES-MS (+ve)) 361 [M+H]⁺, Retention time 0.79 min.

### Example 5

### Stage 1: 6′-Chloro-4-pyridin-4-yl-3,4,5,6-tetrahydro-2H-[1,2′]bipyrazinyl was prepared as for example 1 Stage 1

### Stage 2: 2-(4-Pyridin-4-yl-3,4,5,6-tetrahydro-2H-[1,2′]bipyrazinyl-6′-yl)-benzamide (Comp. 70)

6′-Chloro-4-pyridin-4-yl-3,4,5,6-tetrahydro-2H-[1,2′]bipyrazinyl (50 mg, 0.18 mmol), 2-(aminocarbonylphenyl)boronic acid (41 mg, 0.25 mmol), cesium carbonate (117 mg, 0.36 mmol) and palladium tetrakistriphenylphosphine (8 mg, 0.007 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the reaction mixture filtered through a short pad of celite. The solvent was removed under reduced pressure, the residue washed with diethyl ether, dissolved in chloroform, washed with saturated aqueous sodium carbonate and dried over MgSO₄. The solvent was removed in vacuo and the product was purified by recrystallization from methanol.

Yield: 5.6 mg (9%)

Mass spectrum (ES-MS (+ve)) 361 [M+H]⁺, Retention time 0.83 min.

### Example 6

### Stage 1: 6′-Chloro-4-pyridin-4-yl-3,4,5,6-tetrahydro-2H-[1,2′]bipyrazinyl was Prepared as for Example 1 Stage 1

### Stage 2: 6′-(4-Methylsulfanyl-phenyl)-4-pyridin-4-yl-3,4,5,6-tetrahydro-2H-[1,2′]bipyrazinyl (Compound 66)

6′-Chloro-4-pyridin-4-yl-3,4,5,6-tetrahydro-2H-[1,2′]bipyrazinyl (50 mg, 0.18 mmol), 4-methylthiaphenyl boronic acid (41 mg, 0.20 mmol), cesium carbonate (117 mg, 0.36 mmol) and palladium tetrakistriphenylphosphine (8 mg, 0.007 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the reaction mixture filtered through a short pad of celite. The solvent was removed under reduced pressure, the residue washed with diethyl ether, dissolved in chloroform, washed with saturated aqueous sodium carbonate and dried over MgSO₄. The solvent was removed in vacuo and the product was purified by recrystallization from methanol.

Yield: 39.5 mg (60%)

Mass spectrum (ES-MS (+ve)) 364 [M+H]⁺, Retention time 1.17 min.

### Example 7

### Stage 1: 6′-Chloro-4-pyridin-4-yl-3,4,5,6-tetrahydro-2H-[1,2′]bipyrazinyl was Prepared as for Example 1 Stage 1

### Stage 2: 6′-Phenyl-4-pyridin-4-yl-3,4,5,6-tetrahydro-2H-[1,2′]bipyrazinyl (Compound 67)

6′-Chloro-4-pyridin-4-yl-3,4,5,6-tetrahydro-2H-[1,2′]bipyrazinyl (50 mg, 0.18 mmol), phenyl boronic acid (24 mg, 0.20 mmol), cesium carbonate (117 mg, 0.36 mmol) and palladium tetrakistriphenylphosphine (8 mg, 0.007 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the reaction mixture filtered through a short pad of celite. The solvent was removed under reduced pressure, the residue washed with diethyl ether, dissolved in chloroform, washed with saturated aqueous sodium carbonate and dried over MgSO₄. The solvent was removed in vacuo and the product was purified by recrystallization from methanol.

Yield: 20 mg (35%)

Mass spectrum (ES-MS (+ve)) 318 [M+H]⁺, Retention time 1.07 min.

### Example 8

### Stage 1: 6′-Chloro-4-pyridin-4-yl-3,4,5,6-tetrahydro-2H-[1,2′]bipyrazinyl was Prepared as for Example 1 Stage 1

### Stage 2: 1-[5-(4-Pyridin-4-yl-3,4,5,6-tetrahydro-2H-[1,2′]bipyrazinyl-6′yl)-thiophen-2-yl]-ethanone (Compound 65)

6′-Chloro-4-pyridin-4-yl-3,4,5,6-tetrahydro-2H-[1,2′]bipyrazinyl (50 mg, 0.18 mmol), 5-acetylthiophene-2-boronic acid (34 mg, 0.20 mmol), cesium carbonate (117 mg, 0.36 mmol) and palladium tetrakistriphenylphosphine (8 mg, 0.007 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the reaction mixture filtered through a short pad of celite. The solvent was removed under reduced pressure, the residue washed with diethyl ether, dissolved in chloroform, washed with saturated aqueous sodium carbonate and dried over MgSO₄. The solvent was removed in vacuo and the product was purified by recrystallization from methanol.

Yield: 7.5 mg (11%)

Mass spectrum (ES-MS (+ve)) 366 [M+H]⁺, Retention time 1.07 min.

### Example 9

### Stage 1: 6′-Chloro-4-phenyl-3,4,5,6-tetrahydro-2H-[1,2′]bipyrazinyl

Dichloropyrazine (300 mg, 2.0 mmol), 1-phenylpiperazine (359 mg, 2.2 mmol) and triethylamine (0.420 ml, 3.0 mmol) were dissolved in ethanol (20 ml). The reaction was heated at reflux for 16 h, allowed to reach ambient temperature and the solvent removed in vacuo. The crude material was dissolved in chloroform and washed with saturated aqueous sodium bicarbonate, dried over MgSO₄, filtered, and the solvent removed under reduced pressure. The product was purified by flash column chromatography (EtOAc:hexane, 1:1) to give pure product as a white solid.

Yield: 375 mg (68%)

Mass spectrum (ES-MS (+ve)) 275 [M+H]⁺, Retention time 1.31 min.

¹H-NMR (DMSO-d₆, 400 MHz): δ 8.17 (1H, s, Ar), 7.70 (1H, s, Ar), 7.06 (2H, m, Ar), 6.81 (2H, m, Ar), 6.63 (1H, m, Ar), 3.55 (4H, m, 2×NCH₂), 3.07 (4H, m, 2×NCH₂).

### Stage 2: 4-(4-Phenyl-3,4,5,6-tetrahydro-2H-[1,2′]bipyrazinyl-6′-yl)-benzamide (Comp. 68)

6′-Chloro-4-phenyl-3,4,5,6-tetrahydro-2H-[1,2′]bipyrazinyl (70 mg, 0.25 mmol), 4-(aminocarbonylphenyl)boronic acid (58 mg, 0.35 mmol), cesium carbonate (162 mg, 0.50 mmol) and palladium tetrakistriphenylphosphine (11 mg, 0.01 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the solution filtered through a short pad of celite. The solvent was removed under reduced pressure and the residue washed with diethyl ether and heptane. The product was purified by recrystallization from methanol.

Yield: 44.9 mg (50%)

Mass spectrum (ES-MS (+ve)) 360 [M+H]⁺, Retention time 1.12 min.

¹H-NMR (DMSO-d₆, 400 MHz): δ 8.62 (1H, s, Ar), 8.47 (1H, s, Ar), 8.23 (2H, d, J=8.6 Hz, Ar), 8.16, (1H, br s, NH), 8.05 (2H, d, J=8.6 Hz Ar), 7.54 (1H, br s, NH), 7.31, (2H, dd, J=7.3 Hz, J=8 Hz, Ar), 7.08 (2H, d, J=7.8 Hz, Ar), 6.85 (1H, t, J=7.3 Hz Ar), 3.90 (4H, m, 2×NCH₂), 3.35 (4H, m, 2×NCH₂).

### Example 10

### Stage 1: 6′-Chloro-4-pyridin-2-yl-3,4,5,6-tetrahydro-2H-[1,2′]bipyrazinyl

Dichloropyrazine (300 mg, 2.0 mmol), 1-(2-pyridyl)piperazine (361 mg, 2.2 mmol) and triethylamine (0.420 ml, 3.0 mmol) were dissolved in ethanol (30 ml). The reaction was heated at reflux for 16 h, allowed to reach ambient temperature and the solvent removed in vacuo. The crude material was dissolved in chloroform, washed with saturated aqueous sodium bicarbonate, dried over MgSO₄, filtered, and the solvent removed under reduced pressure. The product was purified by recrystallization from ethanol.

Yield: 280 mg (50%)

Mass spectrum (ES-MS (+ve)) 276 [M+H]⁺, Retention time 0.94 min.

### Stage 2: 4-(4-Pyridin-2-yl-3,4,5,6-tetrahydro-2H-[1,2′]bipyrazinyl-6′-yl)-benzamide (Comp. 15)

6′-Chloro-4-pyridin-2-yl-3,4,5,6-tetrahydro-2H-[1,2′]bipyrazinyl (69 mg, 0.25 mmol), 4-(aminocarbonylphenyl)boronic acid (58 mg, 0.35 mmol), cesium carbonate (162 mg, 0.50 mmol) and palladium tetrakistriphenylphosphine (11 mg, 0.01 mmol). were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the solution filtered through a short pad of celite. The solvent was removed under reduced pressure and the residue washed with diethyl ether and heptane. The crude product was purified by prep-HPLC.

Yield: 47 mg (52%)

Mass spectrum (ES-MS (+ve)) 361 [M+H]⁺, Retention time 0.94 min.

¹H-NMR (DMSO-d₆, 400 MHz): δ 8.54 (1H, s, Ar), 8.38 (1H, s, Ar), 8.16 (H, d, J=8.56, Ar), 8.15 (1H, m, Ar), 8.07, (1H, br s, NH), 7.98 (2H, d, J=8.56, Ar), 7.56 (1H, m, Ar), 7.46 (1H, br s, NH), 6.89 (1H, d, J=8.56, Ar), 6.67 (1H, dd, J=5.14, J=6.85, Ar), 3.79 (4H, m, 2×NCH₂), 3.66 (4H, m, 2×NCH₂).

### Example 11

### Stage 1: 6-Chloro-2-(3,4-dimethoxyphenyl)-3,4,5,6-tetrahydro-2H-[1,2′]bipyrazinyl

Dichloropyrazine (300 mg, 2.0 mmol), 1-(3,4-dimethoxyphenyl)piperazine (490 mg, 2.2 mmol) and triethylamine (0.420 ml, 3.0 mmol) were dissolved in ethanol (30 ml). The reaction was heated at reflux for 16 h, allowed to reach ambient temperature and the solvent removed in vacuo. The crude material was dissolved in chloroform, washed with saturated aqueous sodium bicarbonate, dried over MgSO₄, filtered, and the solvent removed under reduced pressure. The product was purified by recrystallization from ethanol.

Yield: 346 mg (51%)

Mass spectrum (ES-MS (+ve)) 335 [M+H]⁺, Retention time 1.14 min.

### Stage 2: 4-[4-(3,4-Dimethoxyphenyl)-3,4,5,6-tetrahydro-2H-[1,2′]bipyrazinyl-6′-yl)-benzamide (Compound 20)

6-Chloro-2-(3,4-dimethoxyphenyl)-3,4,5,6-tetrahydro-2H-[1,2′]bipyrazinyl (83.5 mg, 0.25 mmol), 4-(aminocarbonylphenyl)boronic acid (58 mg, 0.35 mmol), cesium carbonate (162 mg, 0.50 mmol) and palladium tetrakistriphenylphosphine (11 mg, 0.01 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the solution filtered through a short pad of celite. The solvent was removed under reduced pressure and the residue washed with diethyl ether and heptane. The product was purified by recrystallization from methanol.

Yield: 28 mg (26%)

Mass spectrum (ES-MS (+ve)) 420 [M+H]⁺, Retention time 0.94 min.

### Example 12

### Stage 1: (6-Chloro-pyrazin-2-yl)-[2-(1H-indol-3-yl)-ethyl]-amine

Dichloropyrazine (1.20 g, 8.0 mmol), tryptamine (1.41 g, 8.8 mmol) and triethylamine (1.7 ml, 12.0 mmol) were dissolved in ethanol (50 ml). The reaction was heated at reflux for 16 h, allowed to reach ambient temperature and the solvent reduced in volume by ˜50%. Ethyl acetate was added and unreacted tryptamine precipitated from solution. The precipitate was filtered off and the filtrate evaporated to dryness. Pure product was obtained following flash column chromatography (EtOAc:hexane, 1:1).

Yield: 900 mg (41%)

Mass spectrum (ES-MS (+ve)) 273 [M+H]⁺, Retention time 1.46 min.

### Stage 2: 4-{6-[2-(1H-Indol-3-yl)-ethylamino]-pyrazin-2-yl}-benzamide (Compound 35)

(6-Chloro-pyrazin-2-yl)-[2-(1H-indol-3-yl)-ethyl]-amine (68 mg, 0.25 mmol), 4-(aminocarbonylphenyl)boronic acid (58 mg, 0.35 mmol), cesium carbonate (162 mg, 0.50 mmol) and palladium tetrakistriphenylphosphine (11 mg, 0.01 mmol) were added to a microwave tube and diluted with 5 ml of a mixture of toluene:EtOH (4:1). The tube was sealed and placed in a CEM Discover microwave (power 150 W, temperature 120° C.) for 20 min. The tube was cooled to ambient temperature and the solution filtered through a short pad of celite. The solvent was removed under reduced pressure and the residue washed with diethyl ether and heptane. The product was purified by recrystallization from methanol.

Yield: 13.5 mg (11%)

Mass spectrum (ES-MS (+ve)) 358 [M+H]⁺, Retention time 1.15 min.

### Example 13

The preferred synthesis of the compounds of this invention is illustrated in Scheme II:

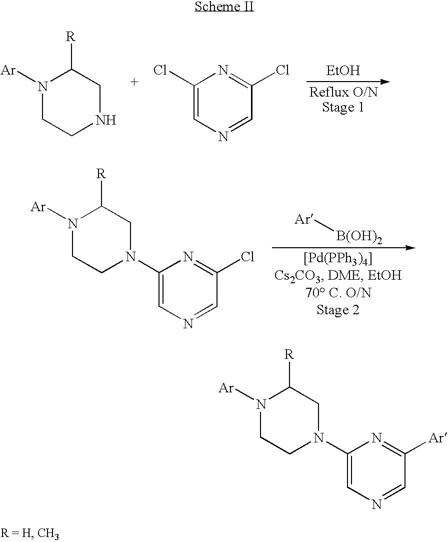

### Stage 1

2,6-Dichloropyrazine (3-4 g, 0.020-0.027 mol, 1 eq), 1-(4-aryl)piperazine (3.8-6.9 g, 1 eq) and triethylamine (2.8-8.3 ml, 1-3 eq) were dissolved in ethanol (16 ml). For 1-(4-aryl)piperazines available as mono- or di-hydrohalide salts two or three equivalents of triethylamine were used. The reaction mixtures were heated at reflux for 16 h, allowed to reach ambient temperature and dried under reduced pressure. The crude, dried material was consecutively washed with 10% aqueous solution of acetic acid, water and saturated aqueous sodium bicarbonate and dried over night under reduced pressure. For the majority of examples workup gave the required product in good purity and intermediates were used directly in stage 2, however for a small number of samples further purification was required either by recrystallisation from EtOH or by column chromatography using ethylacetate/hexane as eluent.

### Stage 2

Stock solutions of pyrazine chlorides (0.15 M), boronic acids (0.18 M) and palladium tetrakistriphenylphosphine (0.007 M) were prepared in a mixture of DME/EtOH 3/1. Aliquots of the pyrazine chloride stock solutions (1.0 ml, 0.15 mmol) were distributed into glass vials containing cesium carbonate (60 mg, 0.18 mmol). Aliquots of boronic acids (1.0 ml, 0.18 mmol) and palladium tetrakistriphenylphosphine (0.4 ml, 0.003 mmol) stock solutions were added to the glass vials and the vials screw capped. The vials were transferred to a Flexchem oven, heated at 70° C. for 16 h and allowed to reach ambient temperature. The reaction mixtures were filtered over celite, transferred into 5 ml 48-well plates and the solvent removed. The dried reaction mixtures were dissolved in 1.6 ml of a mixture of DMSO/CH₃CN 2/1, shaken overnight at ambient temperature and filtered before final purification by preparative HPLC. Fractions of interest were evaporated, resolubilised in CH₃CN and combined into pre-tared vials, analyzed and then dried under reduced pressure.

One illustrative example for the synthesis above, is described below:

### Stage 1 6′-Chloro-4-(3-methoxyphenyl)-4-yl-3,4,5,6-tetrahydro-2H-[1,2′]bipyrazinyl

2,6-Dichloropyrazine (3.00 g, 20.0 mmol), 1-(3-methoxyphenyl)piperazine dihydrochloride (5.30 g, 20.0 mmol) and triethylamine (8.34 ml, 60.0 mmol) were dissolved in ethanol (16 ml). The reaction mixture was heated at reflux for 16 h, allowed to reach ambient temperature and dried under reduced pressure. The crude dried material was consecutively washed with a 10% aqueous solution of acetic acid, water and with saturated aqueous sodium bicarbonate and dried O/N under reduced pressure. Crystallization from EtOH afforded the desired product as a yellow solid.

Yield: 4.40 g (72%)

Mass spectrum (ES-MS (+ve)) 305 [M+H]⁺, Retention time 1.42 min.

¹H-NMR (DMSO-d₆, 400 MHz): δ 8.35 (1H, s, Ar), 7.88 (1H, s, Ar), 7.14 (1H, t, J=8.1 Hz, Ar), 6.58 (1H, ddd, J=8.1 Hz, J=2.4 Hz, J=0.6 Hz, Ar), 6.51 (1H, t, J=2.4 Hz, Ar), 6.41 (1H, ddd, J=8.2 Hz, J=2.4 Hz, J=0.6 Hz, Ar), 3.74-3.71 (7H, m, OCH₃, 2×NCH₂), 3.25 (4H, m, 2×NCH₂).

### Stage 2 6′-(2,5-dimethoxyphenyl)-4-(3-methoxyphenyl)-4-yl-3,4,5,6-tetrahydro-2H-[1,2′]bipyrazinyl

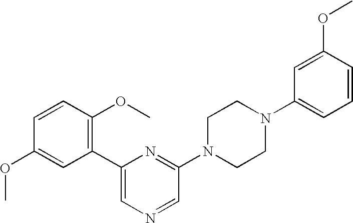

The compound was synthesized according to the general procedure for the preparation of pyrazine library compounds, stage 2 from 6′-Chloro-4-(3-methoxyphenyl)-4-yl-3,4,5,6-tetrahydro-2H-[1,2′]bipyrazinyl and 2,5 dimethoxyphenyl boronic acid.

Yield: 15.8 mg (22%)

Mass spectrum (ES-MS (+ve)) 407 [M+H]⁺, Retention time 1.53 min.

¹H-NMR (DMSO-d₆, 400 MHz): δ 8.56 (1H, s, Ar), 8.45 (1H, s, Ar), 7.40 (1H, d, J=3.2 Hz, Ar), 7.16 (1H, t, J=8.1 Hz, Ar), 7.12 (1H, d, J=9.0 Hz, Ar), 7.03 (1H, dd, J=9.0 Hz, J=3.2 Hz), 6.63 (1H, dd, J=8.1 Hz, J=2.2 Hz, Ar), 6.56 (1H, pt, Ar), 6.43 (1H, dd, J=8.1 Hz, J=2.2 Hz, Ar), 3.83 (3H, s, OCH₃), 3.80-3.77 (7H, m, OCH₃, 2×NCH₂), 3.75 (3H, s, OCH₃), 3.32 (4H, m, 2×NCH₂).

### 2) Materials and Methods:

### General Assay Protocol for all Kinases

(For UL97 a special UL97 kinase assay was used as described below.)

| Reaction Volume: | 40 μl |
|---|---|
| Reaction Time: | 60 min |
| Reaction Temperature: | room temperature |
| Assay Plate: | 96 well U bottom plate (Greiner, 650161) |
| MultiScreen-PH Plate: | 96 well MAPH Filter Plates (Millipore, |
| | MAPHNOB50) |
| Filter Washing Solution: | 0.75% H₃PO₄ |
| Szintilation Liquid: | Supermix Liquid Szintillator (PerkinElmer, |
| | 1200-439) |

### Controls:

| | |
|---|---|
| Negative Control (C−): | 100 mM EDTA, no Inhibitor |
| Positive Control (C+): | no Inhibitor |

### Reaction Buffer:

### 20 mM Tris-HCl, pH 7.5

### 10 mM MgCl2

### 1 mM DTT

### Final Assay Concentrations:

| | |
|---|---|
| Kinase: | Use kinase conc. yielding 10% ATP |
| | turn over. |
| ATP: | 1 μM |
| Adenosine 5′-[γ-³³P]triphosphate: | 12.5 μCi/ml (Amersham Biosciences, |
| | BF1000) |
| Myelin Basic Protein (MBP): | 10 μM (Invitrogen, 13228-010) |

### Pipetting Sequence:

1) Add 8 μl 50 μM MBP in Reaction Buffer to each well of Assay Plate
2) Add 10 μl 500 mM EDTA in H2O to C− wells
3) Add 8 μl 62.5 μCi/ml Adenosine 5′-[γ-³³P]triphosphate+5 μM ATP in Reaction Buffer to each well
4) Add 8 μl 5 fold concentrated inhibitor in 5% DMSO in Reaction Buffer to each well except to C− and C+ wells
5) Add 8 μl 5% DMSO in Reaction Buffer to C− and C+wells
6) Add 8 μl 5 fold concentrated kinase in Reaction Buffer to each well
7) Incubate 1 hr at room temperature
8) Add 10 μl 50 mM EDTA in H2O to each well except to C− wells
9) Prepare MAPH plates by adding 200 μl 0.75% H₃PO₄ to each well
10) Exhaust 0.75% H₃PO₄ using Millipore vacuum station
11) Add 60 μl 0.75% H₃PO₄ to each well of MAPH Filter Plate
12) Transfer 30 μl sample per well from Assay Plate to corresponding well of MAPH Filter Plate
13) Incubate 30 min at room temperature
14) Wash each well of MAPH Filter Plates 3× with 200 μl 0.75% H₃PO₄ using Millipore vacuum station
15) Add 20 μl Szintilation Liquid to each well of MAPH Filter Plate
16) Seal MAPH Filter Plate
17) Store MAPH Filter Plate 30 min in darkness
18) Quantify radioactivity

### UL97 Kinase-Assay on Immobilon Plate

The effect of pyrazine derivatives of this invention was tested on the activity of the viral kinase pUL-97. This kinase is derived from human cytomegalovirus (HCMV) (M. Marschall et al., Journal of General Virology, 2001, 82, 1439-1450). The pUL-97 gene was cloned into a baculovirus vector in order to produce GST (glutathione S-transferase) fusion protein. Insect cells (Sf9) were infected and GST-UL-97 purified via glutathione affinity columns according to standard procedures.

pUL-97 Kinase Reaction:

The pUL-97 kinase reaction was performed as described (M. Marschall et al., Journal of General Virology, 2001, 82, 1439-1450). Briefly, 10 μl Assay buffer (3 μM ATP, 60 μg/ml myelin basic protein (MBP) as substrate), 1.0 μCi γ-[³³P]ATP and 10 μl Basic buffer (20 mM Tris-HCl pH7.5, 0.5 mM MnCl₂, 1 mM DTT (Dithiothreitol)) were given to the test tube before adding various concentrations of pyrazine derivatives. The reaction was started by adding 0.2 μl pUL-97 kinase, purified from infected Sf9-insect cells as described above. The total volume was adjusted with Basic buffer to a final volume of 30 μl. The reaction mix was incubated for 1 hr at 30° C. For negative control, 10 μl 0.1M EDTA (Ethylene Diamine tetraacetate) was added to reaction mix before addition of pUL-97 protein kinase. The reaction was stopped by addition of 10 μl 0.1 M EDTA.

### Measuring Incorporation of Radioactivity:

The Immobilon plate (Millipore) was rinsed with 50 μl methanol/well. Following addition of 100 μl 0.1 M EDTA, 20 μl of each kinase reaction mix was added to one well of the Immobilon plate. Each well was washed 4× with 250 μl 0.75% phosphoric acid and 1× with 50 μl methanol. After addition of 50 μl scintillation cocktail (Roth, Germany) per well incorporation of radioactivity was measured using a Betareader (Wallac) and enzymatic activity calculated.

### HCMV Infection:

To examine the effect of the pyrazine derivatives on HCMV replication, HCMV infections were performed in a cellular system.

Human foreskin fibroblasts (HFFs) were grown to subconfluency in 12 well plates and infected with 0.25 tissue culture infectious doses of HCMV AD-169 carrying a reporter gene (HCMV based antiviral assay). Pyrazine derivatives (stocks in DMSO (Dimethylsulfoxide)) were diluted in culture medium to the concentrations indicated and added to the cells immediately after virus adsorption. The success of infection (reporter gene expressing cells) and the lack of cytotoxicity of the compound (confluent cell layer) was monitored by microscopy. After seven days, cell layers were harvested, lysed and subjected to the automated fluorometry measurement of reporter gene activity. Each panel refers to a determination in quadruplicate (infection in duplicate, lysate preparation and measurement in duplicate).

### Toxicity Assay:

Toxicity of the pyrazine derivatives was measured by incubating HEK 293 cells with 10 μM of each derivative. The corresponding amount of DMSO served as control.

### UL97 In-Cell Activity:

HEK 293 cells were cultivated in 96-well plates and transfected with pcDNA-UL97, pcDNA-UL97k355m (lysine355methionine exchange=inhibitor insensitive) or pcDNA3.1 as a vector control (all transfections in triplicate). Thereafter, GCV (ganciclovir) was added to the culture media at serial concentrations (5, 10, 20, 40, 80 μM) to induce pUL97-dependent cytotoxic effects. Cytotoxicity was quantified after 5d by measuring the colour conversion of culture media at OD560. Inhibition of the UL97 kinase activity was tested by incubation with the indicated substances at 20 μM concentrations, or indolocarbazole (NGICI-I (100 nM) as a pUL97-specific control inhibitor (cf. FIG. 4).

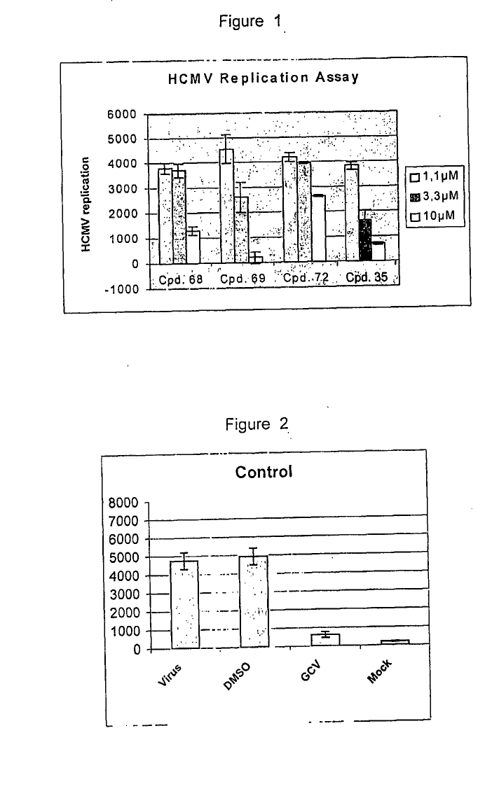

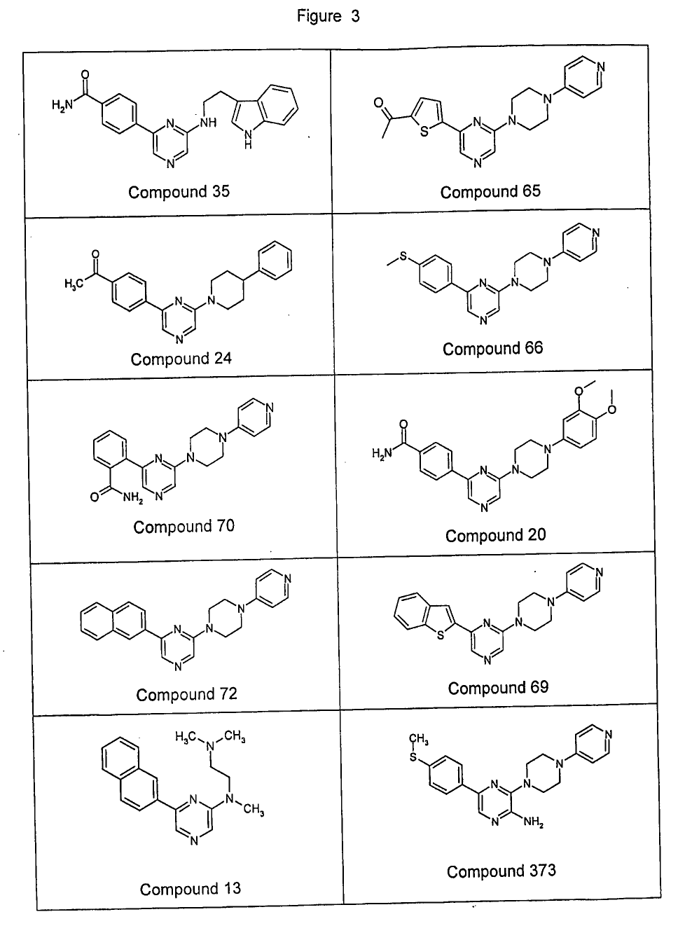

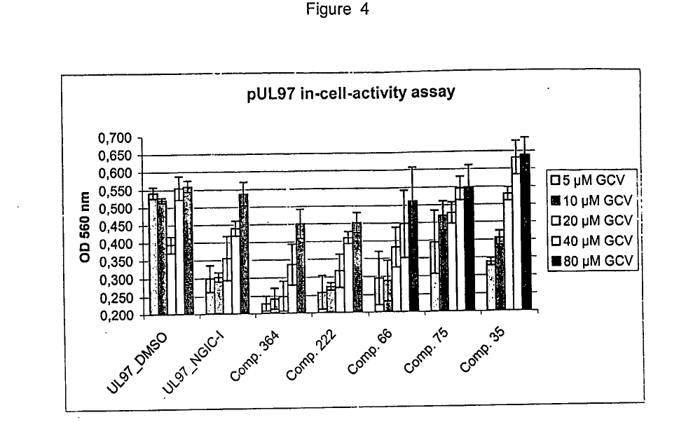

3) Results (see also Table I and II and FIGS. 1 and 2):

The results summarized in Table I and II show, that compounds belonging to the class of pyrazine derivatives have been identified as inhibitors of a broad range of kinases, especially for pUL-97, as well as the tyrosine kinases EGFR, PDGFRbeta, c-Kit and p56Lck, and RICK, SRPK1 and CDK9. Furthermore, compounds belonging to the class of pyrazine derivatives have been identified as inhibitors of HCMV replication in cell culture. Half maximal inhibition constant values (IC₅₀ values) were as low as 3 μM in inhibiting replication of the HCMV strain AD169 in HFF cells and thus were as potent as the standard ganciclovir (IC₅₀ 3-4 μM) (FIG. 1 and FIG. 2).

Additionally, pyrazine derivatives did not show any or low toxicity up to concentrations of 10 μM in HFF cells.

1. Compounds having the general formula (I): 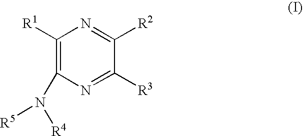
wherein
R¹ and R² are independently selected from the group comprising:
—H, —F, —Cl, —Br, —I, —NH₂, —OH, —SH, —CN, linear or branched, substituted or unsubstituted C₁-C₆ alkyl, linear or branched, substituted or unsubstituted C₂-C₆ alkenyl, linear or branched, substituted or unsubstituted C₂-C₆ alkinyl, substituted or unsubstituted C₃-C₈ cycloalkyl, linear or branched, substituted or unsubstituted C₁-C₆ alkoxy, linear or branched, substituted or unsubstituted C₁-C₆ haloalkyl or linear or branched, substituted or unsubstituted C₁-C₆ thioalkyl;
R³ is selected from substituted or unsubstituted C₃-C₈ cycloalkyl, substituted or unsubstituted aryl, substituted or unsubstituted heteroaryl, or substituted or unsubstituted heterocyclyl;
R⁴ is selected from —H or linear or branched C₁-C₆ alkyl;
R⁵ is selected from the group consisting of:
—H, substituted or unsubstituted, linear or branched C₁-C₆ alkyl, substituted or unsubstituted C₃-C₈ cycloalkyl, substituted or unsubstituted aryl, substituted or unsubstituted heteroaryl, substituted or unsubstituted heterocyclyl,
—(CH₂)ₘ—R⁶
wherein m is selected to be an integer from 0 to 6 and
R⁶ is selected from —H, substituted or unsubstituted C₃-C₈ cycloalkyl, aryl substituted or unsubstituted, substituted or unsubstituted heteroaryl, or substituted or unsubstituted heterocyclyl, if m is selected to be an integer from 1 to 6, at least one, preferably one to two hydrogen atoms bonded to the —(CH₂)ₘ carbon chain are optionally substituted by —F, —Cl, —Br, —I, —OH, —NH₂, linear or branched C₁-C₆ alkyl or linear or branched C₁-C₆ alkoxy,
or —(CH₂)ₙ—N(R⁷)₂
wherein n is an integer from 1 to 6 and R⁷ is selected from —H or linear or branched C₁-C₆ alkyl; if n is selected to be an integer from 1 to 6, at least one, preferably one to two hydrogen atoms bonded to the —(CH₂)ₙ carbon chain are optionally substituted by —F, —Cl, —Br, —I, —OH, —NH₂, linear or branched C₁-C₆ alkyl or linear or branched C₁-C₆ alkoxy
under the proviso, that if R₄ is —H, R₅ is different from —H;
or wherein
R⁴ and R⁵ together form a ring system represented by the formula (II) 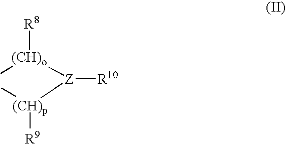
wherein o and p are independently selected to be an integer from 1 to 3,
Z is selected from CH or N,
each R⁸ and each R⁹ represent independently from each other —H, linear or branched C₁-C₆ alkyl or
—(CH₂)ᵤ—OH
wherein u is selected to be an integer from 0 to 6 and if u is selected to be an integer from 1 to 6, at least one, preferably one to two hydrogen atoms bonded to the —(CH₂)ᵤ carbon chain are optionally substituted by —F, —Cl, —Br, —I, —OH, —NH₂, linear or branched C₁-C₆ alkyl or linear or branched C₁-C₆ alkoxy,
R¹⁰ is selected from the group comprising:
—H, linear or branched, substituted or unsubstituted C₁-C₆ alkyl, linear or branched, substituted or unsubstituted C₂-C₆ alkenyl, linear or branched, substituted or unsubstituted C₂-C₆ alkinyl, substituted or unsubstituted C₃-C₈ cycloalkyl, linear or branched, substituted or unsubstituted C₁-C₆ alkoxy, linear or branched, substituted or unsubstituted C₁-C₆ haloalkyl or linear or branched, substituted or unsubstituted C₁-C₆ thioalkyl, substituted or unsubstituted aryl, substituted or unsubstituted heteroaryl or substituted or unsubstituted heterocyclyl,
—C(O)—R¹¹ or —(CH₂)_{q}—R¹¹
wherein q is an integer from 0 to 6 and R₁₁ is selected from linear or branched, substituted or unsubstituted C₁-C₆ alkyl, linear or branched, substituted or unsubstituted C₂-C₆ alkenyl, linear or branched, substituted or unsubstituted C₂-C₆ alkinyl, substituted or unsubstituted C₃-C₈ cycloalkyl, linear or branched, substituted or unsubstituted C₁-C₆ alkoxy, linear or branched, substituted or unsubstituted C₁-C₆ haloalkyl or linear or branched, substituted or unsubstituted C₁-C₆ thioalkyl, substituted or unsubstituted aryl, substituted or unsubstituted heteroaryl, or substituted or unsubstituted heterocyclyl,
and stereoisomeric forms, prodrugs and/or pharmaceutically acceptable salts thereof.

2. The compound according to claim 1, wherein R¹ is selected from —H, linear or branched C₁-C₆ alkyl or —NH₂, preferably from —H, linear or branched C₁-C₄ alkyl or —NH₂, and more preferably from —H, —CH₃ or —NH₂ and most preferably from —H.

3. The compound according to any one of claims 1 to 2, wherein R² is selected from —H, linear or branched C₁-C₆ alkyl or —NH₂, preferably from —H, linear or branched C₁-C₄ alkyl or —NH₂, and more preferably from —H or —CH₃, and most preferably from —H.

4. The compound according to any one of claims 1 to 3, wherein R³ is selected from the group consisting of:
substituted or unsubstituted aryl, substituted or unsubstituted heteroaryl, or substituted or unsubstituted heterocyclyl, and preferably is substituted or unsubstituted aryl or substituted or unsubstituted heteroaryl.

5. The compound according to any one of claims 1 to 4, wherein R⁴ is selected from —H or linear or branched C₁-C₄ alkyl, preferably from —H or —CH₃, and is most preferably —H,
R⁵ is selected from linear or branched C₁-C₆ alkyl, preferably from linear or branched C₁-C₄ alkyl, more preferably from —C₂H₅ or —C₃H₇,
(CH₂)ₘ—R⁶
wherein m is selected to be an integer from 0 to 4, preferably from 0 to 2, and wherein R⁶ is selected from the group comprising: —H, substituted or unsubstituted aryl, preferably substituted or unsubstituted phenyl, 2-thiophenyl, 3-indolyl,
heteroaryl, selected from the group comprising: pyridinyl, pyrrolyl, furanyl, thiophenyl, benzo[b]thiophenyl, benzofuranyl or indolyl, preferably 4-pyridinyl, 2-furanyl,
or —(CH₂)ₙ—N(R⁷)₂,
wherein n is selected to be an integer from 1 to 4, preferably from 1 to 2 and each R⁷ is independently selected from —H or linear or branched C₁-C₄ alkyl, and preferably is —H or —CH₃.

6. The compound according to claim 5, wherein R³ is selected from the group consisting of:
substituted or unsubstituted aryl or substituted or unsubstituted heteroaryl, preferably aryl or bicyclic heteroaryl, more preferably phenyl, naphthyl, benzofuranyl or indolyl, and is most preferably phenyl.

7. The compound according to claim 6, wherein R³, and R⁶ are selected to be phenyl, each independently of the other partially or fully substituted with members selected from the group consisting of:
—F, —Cl, —Br, —I, —OH, linear or branched C₁-C₄ alkoxy, linear or branched C₁-C₄ alkyl, linear or branched C₁-C₄ haloalkyl, —CN, —C(O)—NH—(CH₂)ₛ—N(R¹²)₂ group,
wherein s is an integer from 0 to 4, preferably an integer from 0 to 3, and is most preferably 2 and wherein each R¹² represents independently from each other —H or linear or branched C₁-C₄ alkyl, and preferably is —H or —CH₃ or
—C(O)—R¹³ group
wherein R¹³ is selected to be —N(R¹²)₂
wherein each R¹² represents independently from each other —H or linear or branched C₁-C₄ alkyl, and preferably is —H.

8. The compound according to claim 7, wherein R³ and R⁶ are independently of the other substituted with members selected from the group comprising:
—F, —Cl, —CN, —OH, —OCH₃, —CF₃, —CH₃ or —C(O)NH₂, and wherein each phenyl is preferably mono-, di- or trisubstituted, more preferably mono- or disubstituted.

9. The compound according to any one of claims 1 to 8, wherein R⁵ is selected to be —(CH₂)ₙ—N(R⁷)₂, wherein n is selected to be an integer from 1 to 4, preferably from 1 to 3, and is most preferably 2, and wherein each R⁷ is independently selected from —H, linear or branched C₁-C₄ alkyl, and preferably is —CH₃ and R₃ is selected from aryl or heteroaryl, preferably from aryl, and is most preferably naphthyl.

10. The compound according to claim 1 to 5, wherein R⁴ and R⁵ form a ring system represented by the formula (II) 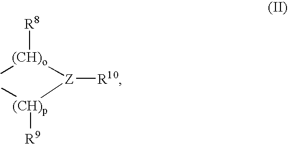
wherein o and p are independently selected to be 1 or 2, preferably o is selected to be 1 or 2 and p is selected to be 2, and most preferably o and p are both selected to be 2,
each R⁸ and each R⁹ is independently selected from the group comprising:
—(CH₂)ᵤ—OH, wherein u is an integer from 0 to 4, preferably from 0 to 2,
—H or linear or branched C₁-C₄ alkyl, preferably —H or —CH₃,
Z is N or CH,
R¹⁰ is selected from the consisting of:
—H, substituted or unsubstituted aryl, substituted or unsubstituted heteroaryl, substituted or unsubstituted heterocyclyl,
—(CH₂)_{q}—R¹¹ or —C(O)—R¹¹
wherein q is selected to be an integer from 0 to 6,
R¹¹ is selected to be a heteroaryl or heterocyclyl, preferably a 5 membered monocyclic heteroaryl or a 5 membered monocyclic heterocyclyl, and is most preferably thiophenyl, furanyl, pyrroyl or pyrrolidinyl.

11. The compound according to claim 10, wherein Z is selected to be CH.

12. The compound according to claim 11, wherein o is selected to be 1 and p is selected to be 2, R⁸ is —H, each R⁹ represents independently from each other —H or —CH₂—OH, R³ is a heteroaryl, preferably a bicyclic heteroaryl, and is most preferably benzofuranyl and R¹⁰ is —H.

13. The compound according to claim 11, wherein o and p are selected to be 2, each R⁸ and each R⁹ are selected to be —H,
R³ is selected to be substituted or unsubstituted aryl or substituted or unsubstituted heteroaryl, and
R¹⁰ is selected to be substituted or unsubstituted aryl, preferably substituted or unsubstituted phenyl.

14. The compound according to claim 13, wherein R³ is selected from the group comprising:
furanyl, thiophenyl, pyrrolidinyl, preferably 3-furanyl or 3-thiophenyl, benzo[b]thiophenyl, benzofuranyl, indolyl, preferably 3-benzo[b]thiophenyl, naphthyl or phenyl, each of these moieties being substituted or unsubstituted with members of the group consisting of:
—F, —Cl, —Br, —I, preferably —F or —Cl,
linear or branched C₁-C₄ alkoxy, preferably —OCH₃,
linear or branched C₁-C₄ thioalkyl, preferably —SCH₃,
—(CH₂)ᵣ—N(R¹²)₂,
wherein r is an integer from 0 to 4, preferably from 0 to 2, and is most preferably 0 and wherein each R¹² represents independently from each other —H or linear or branched C₁-C₄ alkyl, preferably —CH₃,
or
—C(O)—R¹³
wherein R¹³ is selected to be linear or branched C₁-C₄ alkyl, preferably —CH₃.

15. The compound according to claim 10, wherein Z is selected to be N and wherein o and p are independently selected to be an integer from 1 to 3, preferably o and p are selected to be 2, each R⁸ and each R⁹ are independently selected from —H or —CH₃,
R³ is selected from substituted or unsubstituted aryl or substituted or unsubstituted heteroaryl,
and
R¹⁰ is selected from the group comprising:
substituted or unsubstituted aryl, substituted or unsubstituted heteroaryl,
or —C(O)—R¹¹ or —(CH₂)_{q}—R¹¹
wherein R¹¹ is selected from heteroaryl or heterocyclyl and q is an integer from 0 to 4, preferably from 0 to 2.

16. The compound according to claim 15, wherein each R⁸ and one R⁹ represent —H, and one R⁹ represents —CH₃.

17. The compound according to claim 16, wherein R¹⁰ is phenyl, optionally fully or partially substituted, preferably monosubstituted, with linear or branched C₁-C₄ alkyl, preferably —CH₃,
and wherein R³ is selected from the group comprising:
substituted or unsubstituted naphthyl, bicyclic heteroraryl, preferably benzo[b]thiophenyl, or phenyl, preferably mono- or disubstituted, with members of the group consisting of:
linear or branched C₁-C₄ alkyl, preferably —CH₃, linear or branched C₁-C₄ thioalkyl, preferably —S—CH₃ or —CO(NR¹²)₂, wherein each R¹² represents independently from each other —H or linear or branched C₁-C₄ alkyl, preferably —H

18. The compound according to claim 15, wherein each R⁸ and R⁹ is —H.

19. The compound according to claim 18, wherein R¹⁰ is selected from the group consisting of: Phenyl, pyridinyl, pyrimidinyl, pyrazinyl, benzo[b]thiophenyl, benzofuranyl, indolyl,
—C(O)—R¹¹
wherein R¹¹ is a monocyclic heteroaryl, preferably furanyl,
or —(CH₂)_{q}—R¹¹
wherein q is selected to be 2 and R¹¹ is a monocyclic heterocyclyl, preferably pyrrolidinyl.

20. The compound according to claim 19, wherein R¹⁰ is selected from the group comprising: pyridinyl, pyrazinyl, indolyl or phenyl.

21. The compound according to claim 21, wherein R¹⁰ is pyridinyl, preferably 2-pyridinyl or 4-pyridinyl, more preferably 4-pyridinyl or phenyl, which is optionally fully or partially substituted, preferably mono- or disubstituted, with members of the group consisting of:
—F, —Cl, —Br, or —I, preferably —F or —Cl,
linear or branched C₁-C₄ alkoxy, preferably —OCH₃,
linear or branched C₁-C₄ alkyl, preferably —CH₃,
linear or branched C₁-C₄ haloalkyl, preferably —CF₃
—CN or —(CO)—R¹³, wherein R¹³ represents linear or branched C₁-C₄ alkyl, preferably —CH₃.

22. The compound according to any one of claims 18 to 21, wherein R³ is selected from the group consisting of: phenyl, naphthyl, pyrrolyl, thiophenyl, furanyl, preferably 2-thiophenyl, 3-thiophenyl, 2-furanyl or 3-furanyl; pyridinyl, preferably 3-pyridinyl; benzo[b]thiophenyl, benzofuranyl, indolyl, preferably benzo[b]thiophenyl or benzofuranyl and wherein all of these group members are optionally partially or fully substituted.

23. The compound according to claim 22, wherein naphthyl is optionally partially or fully substituted with C₁-C₄ alkoxy, preferably with —OCH₃,
thiophenyl is optionally partially or fully substituted with
—(CO)—R¹³
wherein R¹³ represents linear or branched C₁-C₄ alkyl, preferably —CH₃ and wherein naphthyl or thiophenyl are preferably monosubstituted.

24. The compound according to claim 23, wherein R³ is phenyl, which is optionally partially or fully substituted with members of the group comprising:
—F, —Cl, —Br, —I, —CN, linear or branched C₁-C₄ alkoxy, —OPh, linear or branched C₁-C₄ alkyl, phenyl, linear or branched C₁-C₄ haloalkyl, linear or branched C₁-C₄ thioalkyl,
—(CH₂)ᵣ—X,
wherein X is selected to be —OH or —(NR¹²)₂
wherein r is an integer from 0 to 2, preferably r is 0 or 1 and each R¹² represents independently from each other —H or linear or branched C₁-C₄ alkyl, preferably —H or —CH₃,
—(CO)—R¹³
wherein R¹³ represents linear or branched C₁-C₄ alkyl, preferably —CH₃ or —(NR¹²)₂, wherein each R¹² represents independently from each other —H or linear or branched C₁-C₄ alkyl, preferably —H,
—NH—(CO)—R¹⁴.
wherein R¹⁴ is selected from —H or linear or branched C₁-C₄ alkyl, preferably —CH₃,
or
—C(O)—NH—(CH₂)ₛ—OH, wherein s is selected to be an integer from 0 to 4, and is preferably 2.

25. The compound according to claim 24, wherein the R³-phenyl group is preferably substituted with members selected from the group comprising:
—F, —Cl, —Br, preferably —F or —Cl, —O—CH₃, —O—C₂H₅, —SCH₃, —CH₃, —CH(CH₃)₂, —C(CH₃)₃, —CH₂OH, —N(CH₃)₂, —CF₃ or —C(O)NH₂ and wherein the phenyl is preferably mono-, di- or trisubstituted, more preferably mono- or disubstituted.

26. The compound according to any one of claims 18-25, wherein R¹ and R² are —H.

27. The compound according to any one of the preceding claims, wherein the compound is selected from the group of compounds consisting of:
(Compound 1) 4-(3,4-Dimethoxy-phenyl)-6′-naphthalen-2-yl-3,4,5,6-tetrahydro-2H-[1,2′]bipyrazinyl;
(Compound 2) 6′-(3-Chloro-phenyl)-4-pyridin-4-yl-3,4,5,6-tetrahydro-2H-[1,2′]bipyrazinyl;
(Compound 3) 6′-(4-Isopropyl-phenyl)-4-pyridin-4-yl-3,4,5,6-tetrahydro-2H-[1,2′]bipyrazinyl;
(Compound 4) 3-(4-Pyridin-4-yl-3,4,5,6-tetrahydro-2H-[1,2′]bipyrazinyl-6′-yl)-phenol;
(Compound 5) 4-(3,4-Dimethoxy-phenyl)-6′-(4-isopropyl-phenyl)-3,4,5,6-tetrahydro-2H-[1,2′]bipyrazinyl;
(Compound 6) {4-[4-(3,4-Dimethoxy-phenyl)-3,4,5,6-tetrahydro-2H-[1,2′]bipyrazinyl-6′-yl]-phenyl}-methanol;
(Compound 7) [6-(3-Fluoro-phenyl)-pyrazin-2-yl]-(3,4,5-trimethoxy-phenyl)-amine;
(Compound 8) 6′-(3-Fluoro-phenyl)-4-pyridin-4-yl-3,4,5,6-tetrahydro-2H-[1,2′]bipyrazinyl;
(Compound 9) (6-Naphthalen-2-yl-pyrazin-2-yl)-(2-pyridin-4-yl-ethyl)-amine;
(Compound 10) 6′-Naphthalen-2-yl-4-pyridin-2-yl-3,4,5,6-tetrahydro-2H-[1,2′]bipyrazinyl;
(Compound 11) 4-[6-(3-Chloro-4-fluoro-phenylamino pyrazin-2-yl]-phenol;
(Compound 12) 6′-(4-Methoxy-phenyl)-4-pyridin-4-yl-3,4,5,6-tetrahydro-2H-[1,2′]bipyrazinyl;
(Compound 13) N,N,N′-Trimethyl-N′-(6-naphthalen-2-yl-pyrazin-2-yl)-ethane-1,2-diamine;
(Compound 14) 6′-(4-Chloro-phenyl)-4-pyridin-2-yl-3,4,5,6-tetrahydro-2H-[1,2′]bipyrazinyl;
(Compound 15) 4-(4-Pyridin-2-yl-3,4,5,6-tetrahydro-2H-[1,2′]bipyrazinyl-6′-yl)-benzamide;
(Compound 16) Dimethyl-[3-(4-pyridin-4-yl-3,4,5,6-tetrahydro-2H-[1,2′]bipyrazinyl-6′-yl)-phenyl]-amine;
(Compound 17) 6′-Naphthalen-2-yl-4-(2-pyrrolidin-1-yl-ethyl)-3,4,5,6-tetrahydro-2H-[1,2′]bipyrazinyl;
(Compound 18) [6-(4-Methoxy-phenyl pyrazin-2-yl]-(2-pyridin-3-yl-ethyl)-amine;
(Compound 19) 6′-(2,4-Difluoro-phenyl)-4-pyridin-2-yl-3,4,5,6-tetrahydro-2H-[1,2′]bipyrazinyl;
(Compound 20) 4-[4-(3,4-Dimethoxy-phenyl)-3,4,5,6-tetrahydro-2H-[1,2′]bipyrazinyl-6′-yl]-benzamide;
(Compound 21) 4-(4-Pyridin-4-yl-3,4,5,6-tetrahydro-2H-[1,2′]bipyrazinyl-6′-yl)-benzamide;
(Compound 22) 3-[6-(3-Chloro-4-cyano-phenylamino)-pyrazin-2-yl]-N-(2-dimethylamino-ethyl)-benzamide;
(Compound 23) (6-Benzofuran-2-yl-pyrazin-2-yl)-furan-2-ylmethyl-amine;
(Compound 24) [1-(6-Benzofuran-2-yl-pyrazin-2-yl)-pyrrolidin-2-yl]-methanol;
(Compound 25) 4-(6-Propylamino-pyrazin-2-yl)-benzamide;
(Compound 26) [6-(4-Chloro-phenyl)-pyrazin-2-yl]-furan-2-ylmethyl-amine;
(Compound 27) Benzyl-[6-(4-chloro-phenyl)-pyrazin-2-yl]-amine;
(Compound 28) 4-(6-Benzylamino-pyrazin-2-yl)-benzamide;
(Compound 29) 4-[6-(3-Trifluoromethyl-phenylamino)-pyrazin-2-yl]-phenol;
(Compound 30) 4-{6-[(Thiophen-2-ylmethyl)-amino]-pyrazin-2-yl}-phenol;
(Compound 31) 4-Pyridin-2-yl-6′-thiophen-2-yl-3,4,5,6-tetrahydro-2H-[1,2′]bipyrazinyl;
(Compound 32) 4-Pyridin-4-yl-6′-(3-trifluoromethyl-phenyl)-3,4,5,6-tetrahydro-2H-[1,2′]bipyrazinyl;
(Compound 33) 6′-(3-Chloro-4-fluoro-phenyl)-4-pyridin-4-yl-3,4,5,6-tetrahydro-2H-[1,2′]bipyrazinyl;
(Compound 34) 4-[4-(Furan-2-carbonyl)-3,4,5,6-tetrahydro-2H-[1,2′]bipyrazinyl-6′-yl]-benzamide;
(Compound 35) 4-{6-[2-(1H-Indol-3-yl)-ethylamino]-pyrazin-2-yl}-benzamide;
(Compound 36) N-{3-[4-(1H-Indol-4-yl)-3,4,5,6-tetrahydro-2H-[1,2′]bipyrazinyl-6′-yl]-phenyl}-acetamide;
(Compound 37) 4-(1H-Indol-4-yl)-6′-(3,4,5-trimethoxy-phenyl)-3,4,5,6-tetrahydro-2H-[1,2′]bipyrazinyl;
(Compound 38) 4-(1H-Indol-4-yl)-6′-(4-methoxy-phenyl)-3,4,5,6-tetrahydro-2H-[1,2′]bipyrazinyl;
(Compound 39) N-(2-Hydroxy-ethyl)-3-(4-pyridin-4-yl-3,4,5,6-tetrahydro-2H-[1,2′]bipyrazinyl-6′-yl)-benzamide;
(Compound 40) 4-(6-Ethylamino-pyrazin-2-yl)-benzamide;
(Compound 41) 6′-(2,4-Difluoro-phenyl)-4-(3,4-dimethoxy-phenyl)-3,4,5,6-tetrahydro-2H-[1,2′]bipyrazinyl;
(Compound 42) 2-[6′-(4-Chloro-phenyl)-2,3,5,6-tetrahydro-[1,2′]bipyrazinyl-4-yl]-benzonitrile;
(Compound 43) 4-(3,4-Dimethoxy-phenyl)-6′-naphthalen-1-yl-3,4,5,6-tetrahydro-2H-[1,2′]bipyrazinyl;
(Compound 44) 4-(3,4-Dimethoxy-phenyl)-6′-thiophen-2-yl-3,4,5,6-tetrahydro-2H-[1,2′]bipyrazinyl;
(Compound 45) 4-(3,4-Dimethoxy-phenyl)-6′-(4-ethoxy-phenyl)-3,4,5,6-tetrahydro-2H-[1,2′]bipyrazinyl;
(Compound 46) 6′-(2,4-Difluoro-phenyl)-4-pyridin-4-yl-3,4,5,6-tetrahydro-2H-[1,2′]bipyrazinyl;
(Compound 47) 6′-(4-Chloro-phenyl)-4-pyridin-4-yl-3,4,5,6-tetrahydro-2H-[1,2′]bipyrazinyl;
(Compound 48) [6-(2,4-Difluoro-phenyl)-pyrazin-2-yl]-furan-2-ylmethyl-amine;
(Compound 49) 6′-Phenyl)-4-pyridin-2-yl-3,4,5,6-tetrahydro-2H-[1,2′]bipyrazinyl;
(Compound 50) 6′-(3,4-Dimethyl-phenyl)-4-pyridin-2-yl-3,4,5,6-tetrahydro-2H-[1,2′]bipyrazinyl;
(Compound 51) [6-(3,4-Dimethyl-phenyl)-pyrazin-2-yl]-furan-2-ylmethyl-amine;
(Compound 52) [3-(4-Pyridin-4-yl-3,4,5,6-tetrahydro-2H-[1,2′]bipyrazinyl-6′-yl)-phenyl]-methanol;
(Compound 53) 6′-(3-Chloro-phenyl)-4-pyridin-2-yl-3,4,5,6-tetrahydro-2H-[1,2′]bipyrazinyl;
(Compound 54) 4-Pyridin-4-yl-6′-pyridin-3-yl-3,4,5,6-tetrahydro-2H-[1,2′]bipyrazinyl;
(Compound 55) 6′-(5-Isopropyl-2-methoxy-phenyl)-4-pyridin-4-yl-3,4,5,6-tetrahydro-2H-[1,2′]bipyrazinyl;
(Compound 56) 6′-(3-Fluoro-phenyl)-4-pyridin-2-yl-3,4,5,6-tetrahydro-2H-[1,2′]bipyrazinyl;
(Compound 57) 4-(4-Pyridin-4-yl-3,4,5,6-tetrahydro-2H-[1,2′]bipyrazinyl-6′-yl)-phenol;
(Compound 58) Furan-2-yl-[6′-(3-hydroxymethyl-phenyl)-2,3,5,6-tetrahydro-[1,2′]bipyrazinyl-4-yl]-methanone;
(Compound 59) [6′-(4-Aminomethyl-phenyl)-2,3,5,6-tetrahydro-[1,2′]bipyrazinyl-4-yl]-furan-2-yl-methanone;
(Compound 60) 4-[4-(3,4-Dimethoxy-phenyl)-3,4,5,6-tetrahydro-2H-[1,2′]bipyrazinyl-6′-yl]-benzylamine;
(Compound 61) {4-[4-(1H-Indol-4-yl)-3,4,5,6-tetrahydro-2H-[1,2′]bipyrazinyl-6′-yl]-phenyl}-methanol;
(Compound 62) 2-[6′-(3-Chloro-4-fluoro-phenyl)-2,3,5,6-tetrahydro-[1,2′]bipyrazinyl-4-yl]-benzonitrile;
(Compound 63) {3-[4-(2-Pyrrolidin-1-yl-ethyl)-3,4,5,6-tetrahydro-2H-[1,2′]bipyrazinyl-6′-yl]-phenyl}-methanol;
(Compound 64) 4-(1H-Indol-4-yl)-6′-pyridin-3-yl-3,4,5,6-tetrahydro-2H-[1,2′]bipyrazinyl;
(Compound 65) 1-[5-(4-Pyridin-4-yl-3,4,5,6-tetrahydro-2H-[1,2′]bipyrazinyl-6′-yl)-thiophen-2-yl]-ethanone;
(Compound 66) 6′-(4-Methylsulfanyl-phenyl)-4-pyridin-4-yl-3,4,5,6-tetrahydro-2H-[1,2′]bipyrazinyl;
(Compound 67) 6′-Phenyl-4-pyridin-4-yl-3,4,5,6-tetrahydro-2H-[1,2′]bipyrazinyl;
(Compound 68) 4-(4-Phenyl-3,4,5,6-tetrahydro-2H-[1,2′]bipyrazinyl-6′-yl)-benzamide;
(Compound 69) 6′-Benzo[b]thiophen-2-yl-4-pyridin-4-yl-3,4,5,6-tetrahydro-2H-[1,2′]bipyrazinyl;
(Compound 70) 2-(4-Pyridin-4-yl-3,4,5,6-tetrahydro-2H-[1,2′]bipyrazinyl-6′-yl)-benzamide;
(Compound 71) 3-(4-Pyridin-4-yl-3,4,5,6-tetrahydro-2H-[1,2′]bipyrazinyl-6′-yl)-benzamide;
(Compound 72) 6′-Naphthalen-2-yl-4-pyridin-4-yl-3,4,5,6-tetrahydro-2H-[1,2′]bipyrazinyl;
(Compound 73) 6′-Naphthalen-2-yl-4-pyridin-4-yl-3,4,5,6-tetrahydro-2H-[1,2′]bipyrazinyl-3′-ylamine;
(Compound 74) 4-(3′-Amino-4-pyridin-4-yl-3,4,5,6-tetrahydro-2H-[1,2′]bipyrazinyl-6′-yl)-benzamide;
(Compound 75) 6′-(4-Methylsulfanyl-phenyl)-4-pyridin-4-yl-3,4,5,6-tetrahydro-2H-[1,2′]bipyrazinyl-3′-ylamine;
(Compound 76) 6′-(4-Fluoro-phenyl)-4-pyridin-4-yl-3,4,5,6-tetrahydro-2H-[1,2′]bipyrazinyl;
(Compound 77) 6′-(3,4-Dimethoxy-phenyl)-4-pyridin-4-yl-3,4,5,6-tetrahydro-2H-[1,2′]bipyrazinyl;
(Compound 78) 6′-(3-Methoxy-phenyl)-4-pyridin-4-yl-3,4,5,6-tetrahydro-2H-[1,2′]bipyrazinyl;
(Compound 79) 4-Pyridin-4-yl-6′-thiophen-3-yl-3,4,5,6-tetrahydro-2H-[1,2′]bipyrazinyl;
(Compound 80) 4-(3-Chloro-phenyl)-6′-thiophen-3-yl-3,4,5,6-tetrahydro-2H-[1,2′]bipyrazinyl;
(Compound 81) 4-(3-Chloro-phenyl)-6′-naphthalen-2-yl-3,4,5,6-tetrahydro-2H-[1,2′]bipyrazinyl;
(Compound 82) 4-(3-Chloro-phenyl)-6′-o-tolyl-3,4,5,6-tetrahydro-2H-[1,2′]bipyrazinyl;
(Compound 83) {4-[4-(3-Chloro-phenyl)-3,4,5,6-tetrahydro-2H-[1,2′]bipyrazinyl-6′-yl]-phenyl}-methanol;
(Compound 84) 4-(3-Chloro-phenyl)-6′-furan-3-yl-3,4,5,6-tetrahydro-2H-[1,2′]bipyrazinyl;
(Compound 85) 4-(2-Methoxy-phenyl)-6′-thiophen-3-yl-3,4,5,6-tetrahydro-2H-[1,2′]bipyrazinyl;
(Compound 86) 4-(2-Methoxy-phenyl)-6′-naphthalen-2-yl-3,4,5,6-tetrahydro-2H-[1,2′]bipyrazinyl;
(Compound 87) 4-(2-Methoxy-phenyl)-6′-o-tolyl-3,4,5,6-tetrahydro-2H-[1,2′]bipyrazinyl;
(Compound 88) 6′-(4-tert-Butyl-phenyl)-4-(2-methoxy-phenyl)-3,4,5,6-tetrahydro-2H-[1,2′]bipyrazinyl;
(Compound 89) 4-[4-(3-Chloro-phenyl)-3,4,5,6-tetrahydro-2H-[1,2′]bipyrazinyl-6′-yl]-benzamide;
(Compound 90) 6′-Furan-3-yl-4-(2-methoxy-phenyl)-3,4,5,6-tetrahydro-2H-[1,2′]bipyrazinyl;
(Compound 91) 6′-(5-Chloro-2-methoxy-phenyl)-4-(2-methoxy-phenyl)-3,4,5,6-tetrahydro-2H-[1,2′]bipyrazinyl;
(Compound 92) 6′-(4-Ethoxy-phenyl)-4-(2-methoxy-phenyl)-3,4,5,6-tetrahydro-2H-[1,2′]bipyrazinyl;
(Compound 93) 6′-Naphthalen-2-yl-4-phenyl-3,4,5,6-tetrahydro-2H-[1,2′]bipyrazinyl;
(Compound 94) 4-Phenyl-6′-o-tolyl-3,4,5,6-tetrahydro-2H-[1,2′]bipyrazinyl;
(Compound 95) 6′-(4-tert-Butyl-phenyl)-4-phenyl-3,4,5,6-tetrahydro-2H-[1,2′]bipyrazinyl;
(Compound 96) 6′-(2-Fluoro-biphenyl-4-yl)-4-phenyl-3,4,5,6-tetrahydro-2H-[1,2′]bipyrazinyl;
(Compound 97) 4-(3-Chloro-phenyl)-6′-phenyl-3,4,5,6-tetrahydro-2H-[1,2′]bipyrazinyl;
(Compound 98) 4-(3-Chloro-phenyl)-6′-(3-methoxy-phenyl)-3,4,5,6-tetrahydro-2H-[1,2′]bipyrazinyl;
(Compound 99) 4-(3-Chloro-phenyl)-6′-(4-fluoro-phenyl)-3,4,5,6-tetrahydro-2H-[1,2′]bipyrazinyl;
(Compound 100) 4-(3-Chloro-phenyl)-6′-(3,4-dimethoxy-phenyl)-3,4,5,6-tetrahydro-2H-[1,2′]bipyrazinyl;
(Compound 101) 1-{3-[4-(3-Chloro-phenyl)-3,4,5,6-tetrahydro-2H-[1,2′]bipyrazinyl-6′-yl]-phenyl}-ethanone;
(Compound 102) 4-(3-Chloro-phenyl)-6′-(4-methylsulfanyl-phenyl)-3,4,5,6-tetrahydro-2H-[1,2′]bipyrazinyl;
(Compound 103) 1-{4-[4-(3-Chloro-phenyl)-3,4,5,6-tetrahydro-2H-[1,2′]bipyrazinyl-6′-yl]-phenyl}-ethanone;
(Compound 104) 4-[4-(2-Methoxy-phenyl)-3,4,5,6-tetrahydro-2H-[1,2′]bipyrazinyl-6′-yl]-benzamide;
(Compound 105) 4-(2-Methoxy-phenyl)-6′-(3-methoxy-phenyl)-3,4,5,6-tetrahydro-2H-[1,2′]bipyrazinyl;
(Compound 106) 6′-(4-Fluoro-phenyl)-4-(2-methoxy-phenyl)-3,4,5,6-tetrahydro-2H-[1,2′]bipyrazinyl;
(Compound 107) 6′-(3,4-Dimethoxy-phenyl)-4-(2-methoxy-phenyl)-3,4,5,6-tetrahydro-2H-[1,2′]bipyrazinyl;
(Compound 108) 4-(2-Methoxy-phenyl)-6′-(4-methylsulfanyl-phenyl)-3,4,5,6-tetrahydro-2H-[1,2′]bipyrazinyl;
(Compound 109) 4,6′-Diphenyl-3,4,5,6-tetrahydro-2H-[1,2′]bipyrazinyl;
(Compound 110) 6′-(3-Methoxy-phenyl)-4-phenyl-3,4,5,6-tetrahydro-2H-[1,2′]bipyrazinyl;
(Compound III) 6′-(4-Fluoro-phenyl)-4-phenyl-3,4,5,6-tetrahydro-2H-[1,2′]bipyrazinyl;
(Compound 112) 1-[3-(4-Phenyl-3,4,5,6-tetrahydro-2H-[1,2′]bipyrazinyl-6′-yl)-phenyl]-ethanone;
(Compound 113) 4-(2-Fluoro-phenyl)-6′-(3-methoxy-phenyl)-3,4,5,6-tetrahydro-2H-[1,2′]bipyrazinyl;
(Compound 114) 4-(2-Fluoro-phenyl)-6′-(4-fluoro-phenyl)-3,4,5,6-tetrahydro-2H-[1,2′]bipyrazinyl;
(Compound 115) 1-{3-[4-(2-Fluoro-phenyl)-3,4,5,6-tetrahydro-2H-[1,2′]bipyrazinyl-6′-yl]-phenyl}-ethanone;
(Compound 116) 4-(2-Fluoro-phenyl)-6′-(4-methylsulfanyl-phenyl)-3,4,5,6-tetrahydro-2H-[1,2′]bipyrazinyl;
(Compound 117) [4-(4-Phenyl-3,4,5,6-tetrahydro-2H-[1,2′]bipyrazinyl-6′-yl)-phenyl]-methanol;
(Compound 118) 6′-Furan-3-yl-4-phenyl-3,4,5,6-tetrahydro-2H-[1,2′]bipyrazinyl;
(Compound 119) 6′-(5-Chloro-2-methoxy-phenyl)-4-phenyl-3,4,5,6-tetrahydro-2H-[1,2′]bipyrazinyl;
(Compound 120) 6′-(4-Methylsulfanyl-phenyl)-4-phenyl-3,4,5,6-tetrahydro-2H-[1,2′]bipyrazinyl;
(Compound 121) 6′-(4-Ethoxy-phenyl)-4-phenyl-3,4,5,6-tetrahydro-2H-[1,2′]bipyrazinyl;
(Compound 122) 4-(2-Fluoro-phenyl)-6′-thiophen-3-yl-3,4,5,6-tetrahydro-2H-[1,2′]bipyrazinyl;
(Compound 123) 4-(2-Fluoro-phenyl)-6′-naphthalen-2-yl-3,4,5,6-tetrahydro-2H-[1,2′]bipyrazinyl;
(Compound 124) 4-(2-Fluoro-phenyl)-6′-o-tolyl-3,4,5,6-tetrahydro-2H-[1,2′]bipyrazinyl;
(Compound 125) {4-[4-(2-Fluoro-phenyl)-3,4,5,6-tetrahydro-2H-[1,2′]bipyrazinyl-6′-yl]-phenyl}-methanol;
(Compound 126) 4-(2-Fluoro-phenyl)-6′-furan-3-yl-3,4,5,6-tetrahydro-2H-[1,2′]bipyrazinyl;
(Compound 127) 6′-(4-Ethoxy-phenyl)-4-(2-fluoro-phenyl)-3,4,5,6-tetrahydro-2H-[1,2′]bipyrazinyl;
(Compound 128) 6′-(2-Fluoro-biphenyl-4-yl)-4-(2-fluoro-phenyl)-3,4,5,6-tetrahydro-2H-[1,2′]bipyrazinyl;
(Compound 129) 4-(3-Chloro-phenyl)-6′-(4-ethoxy-phenyl)-3,4,5,6-tetrahydro-2H-[1,2′]bipyrazinyl;
(Compound 130) 1-{4-[4-(2-Fluoro-phenyl)-3,4,5,6-tetrahydro-2H-[1,2′]bipyrazinyl-6′-yl]-phenyl}-ethanone;
(Compound 131) 4-(4-Pyridin-4-yl-3,4,5,6-tetrahydro-2H-[1,2′]bipyrazinyl-6′-yl)-benzonitrile;
(Compound 132) 1-[3-(4-Pyridin-4-yl-3,4,5,6-tetrahydro-2H-[1,2′]bipyrazinyl-6′-yl)-phenyl]-ethanone;
(Compound 133) 1-[4-(4-Pyridin-4-yl-3,4,5,6-tetrahydro-2H-[1,2′]bipyrazinyl-6′-yl)-phenyl]-ethanone;
(Compound 134) 4-(3,4-Dimethoxy-phenyl)-6′-(3-methoxy-phenyl)-3,4,5,6-tetrahydro-2H-[1,2′]bipyrazinyl;
(Compound 135) 6′-Phenyl-4-p-tolyl-3,4,5,6-tetrahydro-2H-[1,2′]bipyrazinyl;
(Compound 136) 6′-(4-Fluoro-phenyl)-4-p-tolyl-3,4,5,6-tetrahydro-2H-[1,2′]bipyrazinyl;
(Compound 137) 6′-(4-Methylsulfanyl-phenyl)-4-p-tolyl-3,4,5,6-tetrahydro-2H-[1,2′]bipyrazinyl;
(Compound 138) 4,6′-Bis-(4-fluoro-phenyl)-3,4,5,6-tetrahydro-2H-[1,2′]bipyrazinyl;
(Compound 139) 6′-(3,4-Dimethoxy-phenyl)-4-(4-fluoro-phenyl)-3,4,5,6-tetrahydro-2H-[1,2′]bipyrazinyl;
(Compound 140) 4-(4-Fluoro-phenyl)-6′-(4-methylsulfanyl-phenyl)-3,4,5,6-tetrahydro-2H-[1,2′]bipyrazinyl;
(Compound 141) 1-{2-[4-(2,4-Difluoro-phenyl)-3,4,5,6-tetrahydro-2H-[1,2′]bipyrazinyl-6′-yl]-phenyl}-ethanone;
(Compound 142) 4-(2,4-Difluoro-phenyl)-6′-p-tolyl-3,4,5,6-tetrahydro-2H-[1,2′]bipyrazinyl;
(Compound 143) 4-(2,4-Difluoro-phenyl)-6′-naphthalen-1-yl-3,4,5,6-tetrahydro-2H-[1,2′]bipyrazinyl;
(Compound 144) 4-(2,4-Difluoro-phenyl)-6′-(2,5-dimethoxy-phenyl)-3,4,5,6-tetrahydro-2H-[1,2′]bipyrazinyl;
(Compound 145) 6′-(3-Chloro-4-fluoro-phenyl)-4-(2,4-difluoro-phenyl)-3,4,5,6-tetrahydro-2H-[1,2′]bipyrazinyl;
(Compound 146) 1-{2-[4-(4-Acetyl-phenyl)-3,4,5,6-tetrahydro-2H-[1,2′]bipyrazinyl-6′-yl]-phenyl}-ethanone;
(Compound 147) 1-[4-(6′-m-Tolyl-2,3,5,6-tetrahydro-[1,2′]bipyrazinyl-4-yl)-phenyl]-ethanone;
(Compound 148) 1-[4-(6′-p-Tolyl-2,3,5,6-tetrahydro-[1,2′]bipyrazinyl-4-yl)-phenyl]-ethanone;
(Compound 149) 1-[4-(6′-Naphthalen-1-yl-2,3,5,6-tetrahydro-[1,2′]bipyrazinyl-4-yl)-phenyl]-ethanone;
(Compound 150) 1-{4-[6′-(2,3-Dimethyl-phenyl)-2,3,5,6-tetrahydro-[1,2′]bipyrazinyl-4-yl]-phenyl}ethanone;
(Compound 151) 1-{4-[6′-(3-Chloro-4-fluoro-phenyl)-2,3,5,6-tetrahydro-[1,2′]bipyrazinyl-4-yl]-phenyl}ethanone;
(Compound 152) 1-[4-(6′-Benzo[b]thiophen-3-yl-2,3,5,6-tetrahydro-[1,2′]bipyrazinyl-4-yl)-phenyl]-ethanone;
(Compound 153) 1-{2-[4-(3-Trifluoromethyl-phenyl)-3,4,5,6-tetrahydro-2H-[1,2′]bipyrazinyl-6′-yl]-phenyl}-ethanone;
(Compound 154) 6′-m-Tolyl-4-(3-trifluoromethyl-phenyl)-3,4,5,6-tetrahydro-2H-[1,2′]bipyrazinyl;
(Compound 155) 6′-p-Tolyl-4-(3-trifluoromethyl-phenyl)-3,4,5,6-tetrahydro-2H-[1,2′]bipyrazinyl;
(Compound 156) 6′-(2,3-Dimethyl-phenyl)-4-(3-trifluoromethyl-phenyl)-3,4,5,6-tetrahydro-2H-[1,2′]bipyrazinyl;
(Compound 157) 6′-(3-Chloro-4-fluoro-phenyl)-4-(3-trifluoromethyl-phenyl)-3,4,5,6-tetrahydro-2H-[1,2′]bipyrazinyl;
(Compound 158) 6′-Benzo[b]thiophen-3-yl-4-(3-trifluoromethyl-phenyl)-3,4,5,6-tetrahydro-2H-[1,2′]bipyrazinyl;
(Compound 159) 1-{4-[6′-(4-Methylsulfanyl-phenyl)-2,3,5,6-tetrahydro-[1,2′]bipyrazinyl-4-yl]-phenyl}ethanone;
(Compound 160) 6′-Benzo[b]thiophen-2-yl-4-(3-trifluoromethyl-phenyl)-3,4,5,6-tetrahydro-2H-[1,2′]bipyrazinyl;
(Compound 161) 6′-(3,4-Dimethoxy-phenyl)-4-(3-trifluoromethyl-phenyl)-3,4,5,6-tetrahydro-2H-[1,2′]bipyrazinyl;
(Compound 162) 1-{3-[4-(3-Trifluoromethyl-phenyl)-3,4,5,6-tetrahydro-2H-[1,2′]bipyrazinyl-6′-yl]-phenyl}-ethanone;
(Compound 163) 6′-(4-Phenoxy-phenyl)-4-pyridin-4-yl-3,4,5,6-tetrahydro-2H-[1,2′]bipyrazinyl;
(Compound 164) 6′-(3,5-Bis-trifluoromethyl-phenyl)-4-pyridin-4-yl-3,4,5,6-tetrahydro-2H-[1,2′]bipyrazinyl;
(Compound 165) 6′-(3,5-Dimethyl-phenyl)-4-pyridin-4-yl-3,4,5,6-tetrahydro-2H-[1,2′]bipyrazinyl;
(Compound 166) 6′-(2-Methylsulfanyl-phenyl)-4-pyridin-4-yl-3,4,5,6-tetrahydro-2H-[1,2′]bipyrazinyl;
(Compound 167) 3-(4-Pyridin-4-yl-3,4,5,6-tetrahydro-2H-[1,2′]bipyrazinyl-6′-yl)-benzonitrile;
(Compound 168) 4-(3,4-Dimethoxy-phenyl)-6′-(4-phenoxy-phenyl)-3,4,5,6-tetrahydro-2H-[1,2′]bipyrazinyl;
(Compound 169) 4-(3,4-Dimethoxy-phenyl)-6′-(6-methoxy-naphthalen-2-yl)-3,4,5,6-tetrahydro-2H-[1,2′]bipyrazinyl;
(Compound 170) 4-(3,4-Dimethoxy-phenyl)-6′-(3,5-dimethyl-phenyl)-3,4,5,6-tetrahydro-2H-[1,2′]bipyrazinyl;
(Compound 171) 4-(3,4-Dimethoxy-phenyl)-6′-(2,3-dimethoxy-phenyl)-3,4,5,6-tetrahydro-2H-[1,2′]bipyrazinyl;
(Compound 172) 4-(3,4-Dimethoxy-phenyl)-6′-(4-trifluoromethyl-phenyl)-3,4,5,6-tetrahydro-2H-[1,2′]bipyrazinyl;
(Compound 173) 3-[4-(3,4-Dimethoxy-phenyl)-3,4,5,6-tetrahydro-2H-[1,2′]bipyrazinyl-6′-yl]-benzonitrile;
(Compound 174) 4-(3,4-Dimethoxy-phenyl)-6′-(2-phenoxy-phenyl)-3,4,5,6-tetrahydro-2H-[1,2′]bipyrazinyl;
(Compound 175) 6′-(2-Ethoxy-phenyl)-4-p-tolyl-3,4,5,6-tetrahydro-2H-[1,2′]bipyrazinyl;
(Compound 176) Dimethyl-[4-(4-p-tolyl-3,4,5,6-tetrahydro-2H-[1,2′]bipyrazinyl-6′-yl)-phenyl]-amine;
(Compound 177) 6′-(3,5-Dimethyl-phenyl)-4-p-tolyl-3,4,5,6-tetrahydro-2H-[1,2′]bipyrazinyl;
(Compound 178) 4-p-Tolyl-6′-(4-trifluoromethyl-phenyl)-3,4,5,6-tetrahydro-2H-[1,2′]bipyrazinyl;
(Compound 179) 6′-(3,5-Dimethyl-phenyl)-4-(4-fluoro-phenyl)-3,4,5,6-tetrahydro-2H-[1,2′]bipyrazinyl;
(Compound 180) 4-(4-Fluoro-phenyl)-6′-(4-trifluoromethyl-phenyl)-3,4,5,6-tetrahydro-2H-[1,2′]bipyrazinyl;
(Compound 181) 4-(4-Fluoro-phenyl)-6′-(3-fluoro-phenyl)-3,4,5,6-tetrahydro-2H-[1,2′]bipyrazinyl;
(Compound 182) {4-[4-(3-Chloro-phenyl)-3,4,5,6-tetrahydro-2H-[1,2′]bipyrazinyl-6′-yl]-phenyl}-dimethyl-amine;
(Compound 183) 4-(3-Chloro-phenyl)-6′-(2,3-dimethoxy-phenyl)-3,4,5,6-tetrahydro-2H-[1,2′]bipyrazinyl;
(Compound 184) 4-(3-Chloro-phenyl)-6′-(4-trifluoromethyl-phenyl)-3,4,5,6-tetrahydro-2H-[1,2′]bipyrazinyl;
(Compound 185) 4-(3-Chloro-phenyl)-6′-(2-phenoxy-phenyl)-3,4,5,6-tetrahydro-2H-[1,2′]bipyrazinyl;
(Compound 186) {4-[4-(2-Methoxy-phenyl)-3,4,5,6-tetrahydro-2H-[1,2′]bipyrazinyl-6′-yl]-phenyl}-dimethyl-amine;
(Compound 187) 6′-(6-Methoxy-naphthalen-2-yl)-4-(2-methoxy-phenyl)-3,4,5,6-tetrahydro-2H-[1,2′]bipyrazinyl;
(Compound 188) 4-(2-Methoxy-phenyl)-6′-(4-trifluoromethyl-phenyl)-3,4,5,6-tetrahydro-2H-[1,2′]bipyrazinyl;
(Compound 189) 6′-(4-Phenoxy-phenyl)-4-phenyl-3,4,5,6-tetrahydro-2H-[1,2′]bipyrazinyl;
(Compound 190) Dimethyl-[4-(4-phenyl-3,4,5,6-tetrahydro-2H-[1,2′]bipyrazinyl-6′-yl)-phenyl]-amine;
(Compound 191) 6′-(3,5-Bis-trifluoromethyl-phenyl)-4-phenyl-3,4,5,6-tetrahydro-2H-[1,2′]bipyrazinyl;
(Compound 192) 6′-(3,5-Dimethyl-phenyl)-4-phenyl-3,4,5,6-tetrahydro-2H-[1,2′]bipyrazinyl;
(Compound 193) 4-Phenyl-6′-(4-trifluoromethyl-phenyl)-3,4,5,6-tetrahydro-2H-[1,2′]bipyrazinyl;
(Compound 194) 6′-(3-Fluoro-phenyl)-4-phenyl-3,4,5,6-tetrahydro-2H-[1,2′]bipyrazinyl;
(Compound 195) {4-[4-(2-Fluoro-phenyl)-3,4,5,6-tetrahydro-2H-[1,2′]bipyrazinyl-6′-yl]-phenyl}-dimethyl-amine;
(Compound 196) 6′-(3,5-Bis-trifluoromethyl-phenyl)-4-(2-fluoro-phenyl)-3,4,5,6-tetrahydro-2H-[1,2′]bipyrazinyl;
(Compound 197) 4-(2-Fluoro-phenyl)-6′-(4-trifluoromethyl-phenyl)-3,4,5,6-tetrahydro-2H-[1,2′]bipyrazinyl;
(Compound 198) 4-(2-Fluoro-phenyl)-6′-(3-fluoro-phenyl)-3,4,5,6-tetrahydro-2H-[1,2′]bipyrazinyl;
(Compound 199) 4-(2,4-Difluoro-phenyl)-6′-naphthalen-2-yl-3,4,5,6-tetrahydro-2H-[1,2′]bipyrazinyl;
(Compound 200) {4-[4-(2,4-Difluoro-phenyl)-3,4,5,6-tetrahydro-2H-[1,2′]bipyrazinyl-6′-yl]-phenyl}-methanol;
(Compound 201) 4-(2,4-Difluoro-phenyl)-6′-furan-3-yl-3,4,5,6-tetrahydro-2H-[1,2′]bipyrazinyl;
(Compound 202) {2-[4-(2,4-Difluoro-phenyl)-3,4,5,6-tetrahydro-2H-[1,2′]bipyrazinyl-6′-yl]-phenyl}-methanol;
(Compound 203) 4-(2,4-Difluoro-phenyl)-6′-(4-ethoxy-phenyl)-3,4,5,6-tetrahydro-2H-[1,2′]bipyrazinyl;
(Compound 204) 1-{4-[6′-(4-tert-Butyl-phenyl)-2,3,5,6-tetrahydro-[1,2′]bipyrazinyl-4-yl]-phenyl}-ethanone;
(Compound 205) 1-[4-(6′-Furan-3-yl-2,3,5,6-tetrahydro-[1,2′]bipyrazinyl-4-yl)-phenyl]-ethanone;
(Compound 206) 1-{4-[6′-(3-Fluoro-4-methoxy-phenyl)-2,3,5,6-tetrahydro-[1,2′]bipyrazinyl-4-yl]-phenyl}-ethanone;
(Compound 207) 1-{4-[6′-(4-Ethoxy-phenyl)-2,3,5,6-tetrahydro-[1,2′]bipyrazinyl-4-yl]-phenyl}-ethanone;
(Compound 208) {4-[4-(2,4-Difluoro-phenyl)-3,4,5,6-tetrahydro-2H-[1,2′]bipyrazinyl-6′-yl]-phenyl}dimethyl-amine;
(Compound 209) 4-(2,4-Difluoro-phenyl)-6′-(3,5-dimethyl-phenyl)-3,4,5,6-tetrahydro-2H-[1,2′]bipyrazinyl;
(Compound 210) 4-(2,4-Difluoro-phenyl)-6′-(4-trifluoromethyl-phenyl)-3,4,5,6-tetrahydro-2H-[1,2′]bipyrazinyl;
(Compound 211) 4-(2,4-Difluoro-phenyl)-6′-(3-fluoro-phenyl)-3,4,5,6-tetrahydro-2H-[1,2′]bipyrazinyl;
(Compound 212) 1-{4-[6′-(2-Ethoxy-phenyl)-2,3,5,6-tetrahydro-[1,2′]bipyrazinyl-4-yl]-phenyl}-ethanone;
(Compound 213) 1-{4-[6′-(6-Methoxy-naphthalen-2-yl)-2,3,5,6-tetrahydro-[1,2′]bipyrazinyl-4-yl]-phenyl}-ethanone;
(Compound 214) 1-{4-[6′-(3-Fluoro-phenyl)-2,3,5,6-tetrahydro-[1,2′]bipyrazinyl-4-yl]-phenyl}-ethanone;
(Compound 215) 1-{4-[6′-(2-Phenoxy-phenyl)-2,3,5,6-tetrahydro-[1,2′]bipyrazinyl-4-yl]-phenyl}-ethanone;
(Compound 216) Dimethyl-{4-[4-(3-trifluoromethyl-phenyl)-3,4,5,6-tetrahydro-2H-[1,2′]bipyrazinyl-6′-yl]-phenyl}-amine;
(Compound 217) 6′-(2-Phenoxy-phenyl)-4-(3-trifluoromethyl-phenyl)-3,4,5,6-tetrahydro-2H-[1,2′]bipyrazinyl;
(Compound 218) 1-[2-(4-Pyridin-4-yl-3,4,5,6-tetrahydro-2H-[1,2′]bipyrazinyl-6′-yl)-phenyl]-ethanone;
(Compound 219) 4-Pyridin-4-yl-6′-m-tolyl-3,4,5,6-tetrahydro-2H-[1,2′]bipyrazinyl;
(Compound 220) 4-Pyridin-4-yl-6′-p-tolyl-3,4,5,6-tetrahydro-2H-[1,2′]bipyrazinyl;
(Compound 221) 6′-Naphthalen-1-yl-4-pyridin-4-yl-3,4,5,6-tetrahydro-2H-[1,2′]bipyrazinyl;
(Compound 222) 6′-Benzo[b]thiophen-3-yl-4-pyridin-4-yl-3,4,5,6-tetrahydro-2H-[1,2′]bipyrazinyl;
(Compound 223) 4-(3,4-Dimethoxy-phenyl)-6′-m-tolyl-3,4,5,6-tetrahydro-2H-[1,2′]bipyrazinyl;
(Compound 224) 4-(3,4-Dimethoxy-phenyl)-6′-p-tolyl-3,4,5,6-tetrahydro-2H-[1,2′]bipyrazinyl;
(Compound 225) 4-(3,4-Dimethoxy-phenyl)-6′-(2,5-dimethoxy-phenyl)-3,4,5,6-tetrahydro-2H-[1,2′]bipyrazinyl;
(Compound 226) 4-(3,4-Dimethoxy-phenyl)-6′-(2,3-dimethyl-phenyl)-3,4,5,6-tetrahydro-2H-[1,2′]bipyrazinyl;
(Compound 227) 6′-Benzo[b]thiophen-3-yl-4-(3,4-dimethoxy-phenyl)-3,4,5,6-tetrahydro-2H-[1,2′]bipyrazinyl;
(Compound 228) 6′-m-Tolyl-4-p-tolyl-3,4,5,6-tetrahydro-2H-[1,2′]bipyrazinyl;
(Compound 229) 4,6′-Di-p-tolyl-3,4,5,6-tetrahydro-2H-[1,2′]bipyrazinyl;
(Compound 230) 6′-Naphthalen-1-yl-4-p-tolyl-3,4,5,6-tetrahydro-2H-[1,2′]bipyrazinyl;
(Compound 231) 6′-Benzo[b]thiophen-3-yl-4-p-tolyl-3,4,5,6-tetrahydro-2H-[1,2′]bipyrazinyl;
(Compound 232) 4-(4-Fluoro-phenyl)-6′-m-tolyl-3,4,5,6-tetrahydro-2H-[1,2′]bipyrazinyl;
(Compound 233) 4-(4-Fluoro-phenyl)-6′-p-tolyl-3,4,5,6-tetrahydro-2H-[1,2′]bipyrazinyl;
(Compound 234) 6′-(2,5-Dimethoxy-phenyl)-4-(4-fluoro-phenyl)-3,4,5,6-tetrahydro-2H-[1,2′]bipyrazinyl;
(Compound 235) 6′-Benzo[b]thiophen-3-yl-4-(4-fluoro-phenyl)-3,4,5,6-tetrahydro-2H-[1,2′]bipyrazinyl;
(Compound 236) 1-{2-[4-(3-Chloro-phenyl)-3,4,5,6-tetrahydro-2H-[1,2′]bipyrazinyl-6′-yl]-phenyl}-ethanone;
(Compound 237) 4-(3-Chloro-phenyl)-6′-p-tolyl-3,4,5,6-tetrahydro-2H-[1,2′]bipyrazinyl;
(Compound 238) 6′-(3-Chloro-4-fluoro-phenyl)-4-(3-chloro-phenyl)-3,4,5,6-tetrahydro-2H-[1,2′]bipyrazinyl;
(Compound 239) 6′-Benzo[b]thiophen-3-yl-4-(3-chloro-phenyl)-3,4,5,6-tetrahydro-2H-[1,2′]bipyrazinyl;
(Compound 240) 1-{2-[4-(2-Methoxy-phenyl)-3,4,5,6-tetrahydro-2H-[1,2′]bipyrazinyl-6′-yl]-phenyl}-ethanone;
(Compound 241) 4-(2-Methoxy-phenyl)-6′-p-tolyl-3,4,5,6-tetrahydro-2H-[1,2′]bipyrazinyl;
(Compound 242) 6′-(3-Chloro-4-fluoro-phenyl)-4-(2-methoxy-phenyl)-3,4,5,6-tetrahydro-2H-[1,2′]bipyrazinyl;
(Compound 243) 6′-Benzo[b]thiophen-3-yl-4-(2-methoxy-phenyl)-3,4,5,6-tetrahydro-2H-[1,2′]bipyrazinyl;
(Compound 244) 4-Phenyl-6′-m-tolyl-3,4,5,6-tetrahydro-2H-[1,2′]bipyrazinyl;
(Compound 245) 4-Phenyl-6′-p-tolyl-3,4,5,6-tetrahydro-2H-[1,2′]bipyrazinyl;
(Compound 246) 6′-Naphthalen-1-yl-4-phenyl-3,4,5,6-tetrahydro-2H-[1,2′]bipyrazinyl;
(Compound 247) 6′-Benzo[b]thiophen-3-yl-4-phenyl-3,4,5,6-tetrahydro-2H-[1,2′]bipyrazinyl;
(Compound 248) 4-(2-Fluoro-phenyl)-6′-p-tolyl-3,4,5,6-tetrahydro-2H-[1,2′]bipyrazinyl;
(Compound 249) 4-(2-Fluoro-phenyl)-6′-naphthalen-1-yl-3,4,5,6-tetrahydro-2H-[1,2′]bipyrazinyl;
(Compound 250) 6′-(2,3-Dimethyl-phenyl)-4-(2-fluoro-phenyl)-3,4,5,6-tetrahydro-2H-[1,2′]bipyrazinyl;
(Compound 251) 6′-Benzo[b]thiophen-3-yl-4-(2-fluoro-phenyl)-3,4,5,6-tetrahydro-2H-[1,2′]bipyrazinyl;
(Compound 252) 6′-Naphthalen-2-yl-4-(3-trifluoromethyl-phenyl)-3,4,5,6-tetrahydro-2H-[1,2′]bipyrazinyl;
(Compound 253) 6′-(4-tert-Butyl-phenyl)-4-(3-trifluoromethyl-phenyl)-3,4,5,6-tetrahydro-2H-[1,2′]bipyrazinyl;
(Compound 254) {4-[4-(3-Trifluoromethyl-phenyl)-3,4,5,6-tetrahydro-2H-[1,2′]bipyrazinyl-6′-yl]-phenyl}-methanol;
(Compound 255) 6′-Furan-3-yl-4-(3-trifluoromethyl-phenyl)-3,4,5,6-tetrahydro-2H-[1,2′]bipyrazinyl;
(Compound 256) 4-Pyridin-4-yl-6′-o-tolyl-3,4,5,6-tetrahydro-2H-[1,2′]bipyrazinyl;
(Compound 257) 6′-(4-tert-Butyl-phenyl)-4-pyridin-4-yl-3,4,5,6-tetrahydro-2H-[1,2′]bipyrazinyl;
(Compound 258) [4-(4-Pyridin-4-yl-3,4,5,6-tetrahydro-2H-[1,2′]bipyrazinyl-6′-yl)-phenyl]-methanol;
(Compound 259) 6′-(5-Chloro-2-methoxy-phenyl)-4-pyridin-4-yl-3,4,5,6-tetrahydro-2H-[1,2′]bipyrazinyl;
(Compound 260) 6′-(4-Ethoxy-phenyl)-4-pyridin-4-yl-3,4,5,6-tetrahydro-2H-[1,2′]bipyrazinyl;
(Compound 261) 4-(3,4-Dimethoxy-phenyl)-6′-thiophen-3-yl-3,4,5,6-tetrahydro-2H-[1,2′]bipyrazinyl;
(Compound 262) 4-(3,4-Dimethoxy-phenyl)-6′-o-tolyl-3,4,5,6-tetrahydro-2H-[1,2′]bipyrazinyl;
(Compound 263) 6′-(4-tert-Butyl-phenyl)-4-(3,4-dimethoxy-phenyl)-3,4,5,6-tetrahydro-2H-[1,2′]bipyrazinyl;
(Compound 264) {2-[4-(3,4-Dimethoxy-phenyl)-3,4,5,6-tetrahydro-2H-[1,2′]bipyrazinyl-6′-yl]-phenyl}-methanol;
(Compound 265) 6′-(3-Fluoro-4-methoxy-phenyl)-4-p-tolyl-3,4,5,6-tetrahydro-2H-[1,2′]bipyrazinyl;
(Compound 266) 6′-(4-Ethoxy-phenyl)-4-p-tolyl-3,4,5,6-tetrahydro-2H-[1,2′]bipyrazinyl;
(Compound 267) 4-(4-Fluoro-phenyl)-6′-naphthalen-2-yl-3,4,5,6-tetrahydro-2H-[1,2′]bipyrazinyl;
(Compound 268) 6′-(2-Fluoro-biphenyl-4-yl)-4-(4-fluoro-phenyl)-3,4,5,6-tetrahydro-2H-[1,2′]bipyrazinyl;
(Compound 269) {4-[4-(4-Fluoro-phenyl)-3,4,5,6-tetrahydro-2H-[1,2′]bipyrazinyl-6′-yl]-phenyl}-methanol;
(Compound 270) 4-(4-Fluoro-phenyl)-6′-furan-3-yl-3,4,5,6-tetrahydro-2H-[1,2′]bipyrazinyl;
(Compound 271) 6′-(4-Ethoxy-phenyl)-4-(4-fluoro-phenyl)-3,4,5,6-tetrahydro-2H-[1,2′]bipyrazinyl;
(Compound 272) 6′-Benzo[b]thiophen-2-yl-4-(2,4-difluoro-phenyl)-3,4,5,6-tetrahydro-2H-[1,2′]bipyrazinyl;
(Compound 273) 1-{3-[4-(2,4-Difluoro-phenyl)-3,4,5,6-tetrahydro-2H-[1,2′]bipyrazinyl-6′-yl]-phenyl}-ethanone;
(Compound 274) 4-(2,4-Difluoro-phenyl)-6′-(4-methylsulfanyl-phenyl)-3,4,5,6-tetrahydro-2H-[1,2′]bipyrazinyl;
(Compound 275) 1-{4-[4-(2,4-Difluoro-phenyl)-3,4,5,6-tetrahydro-2H-[1,2′]bipyrazinyl-6′-yl]-phenyl}-ethanone;
(Compound 276) 1-{4-[6′-(4-Fluoro-phenyl)-2,3,5,6-tetrahydro-[1,2′]bipyrazinyl-4-yl]-phenyl}-ethanone;
(Compound 277) 2-Furan-3-yl-6-(4-phenyl-piperidin-1-yl)-pyrazine;
(Compound 278) 2-(3-Fluoro-4-methoxy-phenyl)-6-(4-phenyl-piperidin-1-yl)-pyrazine;
(Compound 279) 6′-Benzo[b]thiophen-3-yl-3-methyl-4-m-tolyl-3,4,5,6-tetrahydro-2H-[1,2′]bipyrazinyl;
(Compound 280) Dimethyl-{4-[6-(4-phenyl-piperidin-1-yl)-pyrazin-2-yl]-phenyl}-amine;
(Compound 281) 6′-(3-Chloro-4-fluoro-phenyl)-4-(2,4-dimethyl-phenyl)-3,4,5,6-tetrahydro-2H-[1,2′]bipyrazinyl;
(Compound 282) 6′-Benzofuran-2-yl-4-(4-methoxy-phenyl)-3,4,5,6-tetrahydro-2H-[1,2′]bipyrazinyl;
(Compound 283) 6′-Benzo[b]thiophen-3-yl-4-(4-methoxy-phenyl)-3,4,5,6-tetrahydro-2H-[1,2′]bipyrazinyl;
(Compound 284) 4-(3-Methoxy-phenyl)-6′-p-tolyl-3,4,5,6-tetrahydro-2H-[1,2′]bipyrazinyl;
(Compound 285) 6′-Benzofuran-2-yl-4-(3-methoxy-phenyl)-3,4,5,6-tetrahydro-2H-[1,2′]bipyrazinyl;
(Compound 286) 6′-Benzo[b]thiophen-3-yl-4-(3-methoxy-phenyl)-3,4,5,6-tetrahydro-2H-[1,2′]bipyrazinyl;
(Compound 287) 4-(3,4-Dichloro-phenyl)-6′-naphthalen-1-yl-3,4,5,6-tetrahydro-2H-[1,2′]bipyrazinyl;
(Compound 288) 6′-(3,4-Dimethoxy-phenyl)-4-(2,4-dimethyl-phenyl)-3,4,5,6-tetrahydro-2H-[1,2′]bipyrazinyl;
(Compound 289) 1-{5-[4-(2,4-Dimethyl-phenyl)-3,4,5,6-tetrahydro-2H-[1,2′]bipyrazinyl-6′-yl]-thiophen-2-yl}-ethanone;
(Compound 290) 4-[4-(4-Methoxy-phenyl)-3,4,5,6-tetrahydro-2H-[1,2′]bipyrazinyl-6′-yl]-benzamide;
(Compound 291) 4-(4-Methoxy-phenyl)-6′-phenyl-3,4,5,6-tetrahydro-2H-[1,2′]bipyrazinyl;
(Compound 292) 6′-Benzo[b]thiophen-2-yl-4-(4-methoxy-phenyl)-3,4,5,6-tetrahydro-2H-[1,2′]bipyrazinyl;
(Compound 293) 4-[4-(4-Methoxy-phenyl)-3,4,5,6-tetrahydro-2H-[1,2′]bipyrazinyl-6′-yl]-benzonitrile;
(Compound 294) 4-(4-Methoxy-phenyl)-6′-(3-methoxy-phenyl)-3,4,5,6-tetrahydro-2H-[1,2′]bipyrazinyl;
(Compound 295) 6′-Benzo[b]thiophen-2-yl-4-(2,3-dimethyl-phenyl)-3,4,5,6-tetrahydro-2H-[1,2′]bipyrazinyl;
(Compound 296) 1-{5-[4-(2,3-Dimethyl-phenyl)-3,4,5,6-tetrahydro-2H-[1,2′]bipyrazinyl-6′-yl]-thiophen-2-yl}-ethanone;
(Compound 297) 1-{3-[4-(2,3-Dimethyl-phenyl)-3,4,5,6-tetrahydro-2H-[1,2′]bipyrazinyl-6′-yl]-phenyl}-ethanone;
(Compound 298) 1-{4-[4-(2,3-Dimethyl-phenyl)-3,4,5,6-tetrahydro-2H-[1,2′]bipyrazinyl-6′-yl]-phenyl}-ethanone;
(Compound 299) 4-(3-Methyl-4-m-tolyl-3,4,5,6-tetrahydro-2H-[1,2′]bipyrazinyl-6′-yl)-benzamide;
(Compound 300) 6′-Benzo[b]thiophen-2-yl-3-methyl-4-m-tolyl-3,4,5,6-tetrahydro-2H-[1,2′]bipyrazinyl;
(Compound 301) 3-Methyl-6′-(4-methylsulfanyl-phenyl)-4-m-tolyl-3,4,5,6-tetrahydro-2H-[1,2′]bipyrazinyl;
(Compound 302) 4-(4-Chloro-phenyl)-6′-(3-methoxy-phenyl)-3,4,5,6-tetrahydro-2H-[1,2′]bipyrazinyl;
(Compound 303) 4-(4-Pyridin-2-yl-3,4,5,6-tetrahydro-2H-[1,2′]bipyrazinyl-6′-yl)-benzonitrile;
(Compound 304) 6′-(3-Methoxy-phenyl)-4-pyridin-2-yl-3,4,5,6-tetrahydro-2H-[1,2′]bipyrazinyl;
(Compound 305) 6′-(4-Fluoro-phenyl)-4-pyridin-2-yl-3,4,5,6-tetrahydro-2H-[1,2′]bipyrazinyl;
(Compound 306) 6′-(3,4-Dimethoxy-phenyl)-4-pyridin-2-yl-3,4,5,6-tetrahydro-2H-[1,2′]bipyrazinyl;
(Compound 307) 1-[3-(4-Pyridin-2-yl-3,4,5,6-tetrahydro-2H-[1,2′]bipyrazinyl-6′-yl)-phenyl]-ethanone;
(Compound 308) 1-[4-(4-Pyridin-2-yl-3,4,5,6-tetrahydro-2H-[1,2′]bipyrazinyl-6′-yl)-phenyl]-ethanone;
(Compound 309) 6′-(4-Fluoro-phenyl)-4-(4-trifluoromethyl-phenyl)-3,4,5,6-tetrahydro-2H-[1,2′]bipyrazinyl;
(Compound 310) 6′-(3,4-Dimethoxy-phenyl)-4-(4-trifluoromethyl-phenyl)-3,4,5,6-tetrahydro-2H-[1,2′]bipyrazinyl;
(Compound 311) 6′-(4-Methylsulfanyl-phenyl)-4-(4-trifluoromethyl-phenyl)-3,4,5,6-tetrahydro-2H-[1,2′]bipyrazinyl;
(Compound 312) 1-[3-(2′,3′,5′,6′-Tetrahydro-[2,1′;4′,2″]terpyrazin-6-yl)-phenyl]-ethanone;
(Compound 313) 6-(4-Bromo-phenyl)-2′,3′,5′,6′-tetrahydro-[2,1′;4′,2″]terpyrazine;
(Compound 314) 6-(4-Methylsulfanyl-phenyl)-2′,3′,5′,6′-tetrahydro-[2,1′;4′,2″]terpyrazine;
(Compound 315) {4-[4-(3,4-Dichloro-phenyl)-3,4,5,6-tetrahydro-2H-[1,2′]bipyrazinyl-6′-yl]-phenyl}-methanol;
(Compound 316) 6′-(3-Chloro-4-fluoro-phenyl)-4-(3,4-dimethyl-phenyl)-3,4,5,6-tetrahydro-2H-[1,2′]bipyrazinyl;
(Compound 317) 6′-Benzo[b]thiophen-3-yl-4-(3,4-dimethyl-phenyl)-3,4,5,6-tetrahydro-2H-[1,2′]bipyrazinyl;
(Compound 318) 2-Naphthalen-1-yl-6-(4-phenyl-piperidin-1-yl)-pyrazine;
(Compound 319) 2-Benzo[b]thiophen-3-yl-6-(4-phenyl-piperidin-1-yl)-pyrazine;
(Compound 320) 4-(2,4-Dimethyl-phenyl)-6′-naphthalen-2-yl-3,4,5,6-tetrahydro-2H-[1,2′]bipyrazinyl;
(Compound 321) {4-[4-(2,4-Dimethyl-phenyl)-3,4,5,6-tetrahydro-2H-[1,2′]bipyrazinyl-6′-yl]-phenyl}-methanol;
(Compound 322) 4-(3-Methoxy-phenyl)-6′-naphthalen-2-yl-3,4,5,6-tetrahydro-2H-[1,2′]bipyrazinyl;
(Compound 323) {4-[4-(3-Methoxy-phenyl)-3,4,5,6-tetrahydro-2H-[1,2′]bipyrazinyl-6′-yl]-phenyl}-methanol;
(Compound 324) 6′-Furan-3-yl-4-(3-methoxy-phenyl)-3,4,5,6-tetrahydro-2H-[1,2′]bipyrazinyl;
(Compound 325) 4-(4-Chloro-phenyl)-6′-m-tolyl-3,4,5,6-tetrahydro-2H-[1,2′]bipyrazinyl;
(Compound 326) 6′-Benzo[b]thiophen-3-yl-4-(4-chloro-phenyl)-3,4,5,6-tetrahydro-2H-[1,2′]bipyrazinyl;
(Compound 327) 4-Pyridin-2-yl-6′-m-tolyl-3,4,5,6-tetrahydro-2H-[1,2′]bipyrazinyl;
(Compound 328) 4-Pyridin-2-yl-6′-p-tolyl-3,4,5,6-tetrahydro-2H-[1,2′]bipyrazinyl;
(Compound 329) 6-m-Tolyl-2′,3′,5′,6′-tetrahydro-[2,1′;4′,2″]terpyrazine;
(Compound 330) 6-p-Tolyl-2′,3′,5′,6′-tetrahydro-[2,1′;4′,2″]terpyrazine;
(Compound 331) 6-Naphthalen-1-yl-2′,3′,5′,6′-tetrahydro-[2,1′;4′,2″]terpyrazine;
(Compound 332) 6-Benzo[b]thiophen-3-yl-2′,3′,5′,6′-tetrahydro-[2,1′;4′,2″]terpyrazine;
(Compound 333) 6-(4-tert-Butyl-phenyl)-2′,3′,5′,6′-tetrahydro-[2,1′;4′,2″]terpyrazine;
(Compound 334) 6-(2-Fluoro-biphenyl-4-yl)-2′,3′,5′,6′-tetrahydro-[2,1′;4′,2″]terpyrazine;
(Compound 335) 4-(3,4-Dimethyl-phenyl)-6′-naphthalen-2-yl-3,4,5,6-tetrahydro-2H-[1,2′]bipyrazinyl;
(Compound 336) 2-(4-Phenyl-piperidin-1-yl)-6-thiophen-3-yl-pyrazine;
(Compound 337) 2-Naphthalen-2-yl-6-(4-phenyl-piperidin-1-yl)-pyrazine;
(Compound 338) 4-(2,3-Dimethyl-phenyl)-6′-(3,5-dimethyl-phenyl)-3,4,5,6-tetrahydro-2H-[1,2′]bipyrazinyl;
(Compound 339) 6′-(3,5-Dimethyl-phenyl)-3-methyl-4-m-tolyl-3,4,5,6-tetrahydro-2H-[1,2′]bipyrazinyl;
(Compound 340) {4-[4-(4-Chloro-phenyl)-3,4,5,6-tetrahydro-2H-[1,2′]bipyrazinyl-6′-yl]-phenyl}-dimethyl-amine;
(Compound 341) 6′-(4-Phenoxy-phenyl)-4-pyridin-2-yl-3,4,5,6-tetrahydro-2H-[1,2′]bipyrazinyl;
(Compound 342) 6′-(2-Methylsulfanyl-phenyl)-4-pyridin-2-yl-3,4,5,6-tetrahydro-2H-[1,2′]bipyrazinyl;
(Compound 343) Dimethyl-[4-(2′,3′,5′,6′-tetrahydro-[2,1′;4′,2″]terpyrazin-6-yl)-phenyl]-amine;
(Compound 344) 6-(3,5-Dimethyl-phenyl)-2′,3′,5′,6′-tetrahydro-[2,1′;4′,2″]terpyrazine;
(Compound 345) 6-(4-Trifluoromethyl-phenyl)-2′,3′,5′,6′-tetrahydro-[2,1′;4′,2″]terpyrazine;
(Compound 346) 4-(2,3-Dimethyl-phenyl)-6′-furan-3-yl-3,4,5,6-tetrahydro-2H-[1,2′]bipyrazinyl;
(Compound 347) 3-Methyl-6′-naphthalen-2-yl-4-m-tolyl-3,4,5,6-tetrahydro-2H-[1,2′]bipyrazinyl;
(Compound 348) 4-(4-Chloro-phenyl)-6′-naphthalen-2-yl-3,4,5,6-tetrahydro-2H-[1,2′]bipyrazinyl;
(Compound 349) 4-(4-Chloro-phenyl)-6′-furan-3-yl-3,4,5,6-tetrahydro-2H-[1,2′]bipyrazinyl;
(Compound 350) 4-Pyridin-2-yl-6′-thiophen-3-yl-3,4,5,6-tetrahydro-2H-[1,2′]bipyrazinyl;
(Compound 351) [4-(4-Pyridin-2-yl-3,4,5,6-tetrahydro-2H-[1,2′]bipyrazinyl-6′-yl)-phenyl]-methanol;
(Compound 352) 6′-Naphthalen-2-yl-4-(4-trifluoromethyl-phenyl)-3,4,5,6-tetrahydro-2H-[1,2′]bipyrazinyl;
(Compound 353) 6′-Furan-3-yl-4-(4-trifluoromethyl-phenyl)-3,4,5,6-tetrahydro-2H-[1,2′]bipyrazinyl;
(Compound 354) 4-(2,4-Dimethyl-phenyl)-6′-(4-trifluoromethyl-phenyl)-3,4,5,6-tetrahydro-2H-[1,2′]bipyrazinyl;
(Compound 355) 4-(4-Methoxy-phenyl)-6′-(4-trifluoromethyl-phenyl)-3,4,5,6-tetrahydro-2H-[1,2′]bipyrazinyl;
(Compound 356) 4-(3-Methoxy-phenyl)-6′-(4-trifluoromethyl-phenyl)-3,4,5,6-tetrahydro-2H-[1,2′]bipyrazinyl;
(Compound 357) {4-[4-(3,4-Dichloro-phenyl)-3,4,5,6-tetrahydro-2H-[1,2′]bipyrazinyl-6′-yl]-phenyl}-dimethyl-amine;
(Compound 358) 4-(3,4-Dichloro-phenyl)-6′-(3,5-dimethyl-phenyl)-3,4,5,6-tetrahydro-2H-[1,2′]bipyrazinyl;
(Compound 359) 4-(3,4-Dimethyl-phenyl)-6′-(4-methylsulfanyl-phenyl)-3,4,5,6-tetrahydro-2H-[1,2′]bipyrazinyl;
(Compound 360) 2-(3,4-Dimethoxy-phenyl)-6-(4-phenyl-piperidin-1-yl)-pyrazine;
(Compound 361) 2-(4-Methylsulfanyl-phenyl)-6-(4-phenyl-piperidin-1-yl)-pyrazine;
(Compound 362) 1-{4-[6-(4-Phenyl-piperidin-1-yl pyrazin-2-yl]-phenyl}-ethanone;
(Compound 363) 6′-(4-Fluoro-phenyl)-4-(4-methoxy-phenyl)-3,4,5,6-tetrahydro-2H-[1,2′]bipyrazinyl;
(Compound 364) 4-[4-(3-Methoxy-phenyl)-3,4,5,6-tetrahydro-2H-[1,2′]bipyrazinyl-6′-yl]-benzamide;
(Compound 365) 4-(3-Methoxy-phenyl)-6′-phenyl-3,4,5,6-tetrahydro-2H-[1,2′]bipyrazinyl;
(Compound 366) 4,6′-Bis-(3-methoxy-phenyl)-3,4,5,6-tetrahydro-2H-[1,2′]bipyrazinyl;
(Compound 367) 6′-(4-Fluoro-phenyl)-4-(3-methoxy-phenyl)-3,4,5,6-tetrahydro-2H-[1,2′]bipyrazinyl;
(Compound 368) 1-{3-[4-(3-Methoxy-phenyl)-3,4,5,6-tetrahydro-2H-[1,2′]bipyrazinyl-6′-yl]-phenyl}-ethanone;
(Compound 369) 4-(3-Methoxy-phenyl)-6′-(4-methylsulfanyl-phenyl)-3,4,5,6-tetrahydro-2H-[1,2′]bipyrazinyl;
(Compound 370) 4-(3,4-Dichloro-phenyl)-6′-(4-fluoro-phenyl)-3,4,5,6-tetrahydro-2H-[1,2′]bipyrazinyl;
(Compound 371) 4-(3,4-Dichloro-phenyl)-6′-(3,4-dimethoxy-phenyl)-3,4,5,6-tetrahydro-2H-[1,2′]bipyrazinyl;
(Compound 372) 4-(3,4-Dichloro-phenyl)-6′-(4-methylsulfanyl-phenyl)-3,4,5,6-tetrahydro-2H-[1,2′]bipyrazinyl;
(Compound 373) 6′-Benzo[b]thiophen-2-yl-3′,5′-dimethyl-4-pyridin-4-yl-3,4,5,6-tetrahydro-2H-[1,2′]bipyrazinyl;
(Compound 374) [6-(3-Amino-phenyl)-pyrazin-2-yl]-(3,4,5-trimethoxy-phenyl)-amine;
(Compound 375) N-{3-[6-(3,4,5-Trimethoxy-phenylamino)-pyrazin-2-yl]-phenyl}-acetamide;
(Compound 376) [6-(4-Isopropyl-phenyl)-pyrazin-2-yl]-(3,4,5-trimethoxy-phenyl)-amine;
(Compound 377) [6-(4-Methoxy-phenyl)-pyrazin-2-yl]-(2,2,3,3-tetrafluoro-2,3-dihydro-benzo[1,4]dioxin-6-yl)-amine;
(Compound 378) 3-[6-(2,2,3,3-Tetrafluoro-2,3-dihydro-benzo[1,4]dioxin-6-ylamino)-pyrazin-2-yl]-phenol;
(Compound 379) [6-(3-Amino-phenyl)-pyrazin-2-yl]-(3-chloro-phenyl)-amine;
(Compound 380) [6-(3-Amino-phenyl)-pyrazin-2-yl]-(4-chloro-phenyl)-amine;
(Compound 381) 3-[6-(4-Chloro-phenylamino)-pyrazin-2-yl]-N-(2-dimethylamino-ethyl)-benzamide;
(Compound 382) (3-Chloro-4-fluoro-phenyl)-[6-(3,4,5-trimethoxy-phenyl)-pyrazin-2-yl]-amine;
(Compound 383) 3-[6-(3-Chloro-4-fluoro-phenylamino)-pyrazin-2-yl]-N-(2-dimethylamino-ethyl)-benzamide;
(Compound 384) [6-(4-Methoxy-phenyl)-pyrazin-2-yl]-(3-trifluoromethyl-phenyl)-amine;
(Compound 385) (3-Chloro-phenyl)-[6-(3-chloro-phenyl)-pyrazin-2-yl]-amine;
(Compound 386) 4-(6-Phenylamino-pyrazin-2-yl)-phenol;
(Compound 387) N-{3-[6-(4-Chloro-phenylamino)-pyrazin-2-yl]-phenyl}-acetamide;
(Compound 388) 3-[6-(4-Methoxy-phenyl)-pyrazin-2-ylamino]-benzonitrile;
(Compound 389) 3-[6-(4-Amino-phenyl)-pyrazin-2-ylamino]-benzonitrile;
(Compound 390) [6-(3-Dimethylamino-phenyl)-pyrazin-2-yl]-(3,4,5-trimethoxy-phenyl)-amine;
(Compound 391) 3-[6-(3-Dimethylamino-phenyl)-pyrazin-2-ylamino]-benzonitrile;
(Compound 392) [6-(4-Ethoxy-phenyl)-pyrazin-2-yl]-(3,4,5-trimethoxy-phenyl)-amine;
(Compound 393) [6-(4-Amino-phenyl)-pyrazin-2-yl]-(3-trifluoromethyl-phenyl)-amine;
(Compound 394) (4-Chloro-phenyl)-[6-(3-dimethylamino-phenyl)-pyrazin-2-yl]-amine;
(Compound 395) [6-(3-Dimethylamino-phenyl)-pyrazin-2-yl]-phenyl-amine;
(Compound 396) 4-[6-(3,4,5-Trimethoxy-phenylamino)-pyrazin-2-yl]-phenol;
(Compound 397) N-{4-[6-(4-Chloro-phenylamino)-pyrazin-2-yl]-phenyl}-acetamide;
(Compound 398) [6-(4-Amino-phenyl)-pyrazin-2-yl]-(3,4,5-trimethoxy-phenyl)-amine;
(Compound 399) N-{4-[6-(3-Chloro-4-fluoro-phenylamino)-pyrazin-2-yl]-phenyl}-acetamide;
(Compound 400) [3-(6-Phenylamino-pyrazin-2-yl)-phenyl]-methanol;
(Compound 401) N-[4-(6-Phenylamino-pyrazin-2-yl)-phenyl]-acetamide;
(Compound 402) (6-Naphthalen-2-yl-pyrazin-2-yl)-(3,4,5-trimethoxy-phenyl)-amine;
(Compound 403) N-{3-[6-(2,2,3,3-Tetrafluoro-2,3-dihydro-benzo[1,4]dioxin-6-ylamino)-pyrazin-2-yl]-phenyl}-acetamide;
(Compound 404) 3-[6-(3-Chloro-phenylamino)-pyrazin-2-yl]-N-(2-dimethylamino-ethyl)-benzamide;
(Compound 405) N-{3-[6-(3-Chloro-4-fluoro-phenylamino)-pyrazin-2-yl]-phenyl}-acetamide;
(Compound 406) 4-(6-Phenylamino-pyrazin-2-yl)-benzamide;
(Compound 407) [6-(2,4-Difluoro-phenyl)-pyrazin-2-yl]-(3,4,5-trimethoxy-phenyl)-amine;
(Compound 408) [6-(4-Amino-phenyl)-pyrazin-2-yl]-(3-chloro-phenyl)-amine;
(Compound 409) N-{5-[6-(3-Dimethylamino-phenyl)-pyrazin-2-ylamino]-2-methyl-phenyl}-methanesulfonamide;
(Compound 410) N-{5-[6-(2,4-Difluoro-phenyl)-pyrazin-2-ylamino]-2-methyl-phenyl}-methanesulfonamide;
(Compound 411) [6-(4-Ethoxy-phenyl)-pyrazin-2-yl]-(3-trifluoromethyl-phenyl)-amine;
(Compound 412) 4-[6-(3-Chloro-phenylamino)-pyrazin-2-yl]-benzamide;
(Compound 413) 4-[6-(4-Amino-phenyl)-pyrazin-2-ylamino]-benzonitrile;
(Compound 414) 4-[6-(3,4,5-Trimethoxy-phenylamino)-pyrazin-2-yl]-benzamide;
(Compound 415) N-{3-[6-(3-Chloro-phenylamino)-pyrazin-2-yl]-phenyl}-acetamide;
(Compound 416) N-[3-(6-Phenylamino-pyrazin-2-yl)-phenyl]-acetamide;
(Compound 417) 3-[6-(3-Chloro-4-fluoro-phenylamino)-pyrazin-2-yl]-phenol;
(Compound 418) N-{3-[6-(3-Trifluoromethyl-phenylamino)-pyrazin-2-yl]-phenyl}-acetamide;
(Compound 419) (4-Chloro-phenyl)-[6-(3,4,5-trimethoxy-phenyl)-pyrazin-2-yl]-amine;
(Compound 420) {2-[6-(4-Chloro-phenylamino)-pyrazin-2-yl]-phenyl}-methanol;
(Compound 421) 3-[6-(3-Amino-phenyl)-pyrazin-2-ylamino]-benzonitrile;
(Compound 422) 3-[6-(5-Isopropyl-2-methoxy-phenyl)-pyrazin-2-ylamino]-benzonitrile;
(Compound 423) 3-[6-(3,4,5-Trimethoxy-phenylamino)-pyrazin-2-yl]-phenol;
(Compound 424) N-[2-Methyl-5-(6-naphthalen-2-yl-pyrazin-2-ylamino)-phenyl]-methanesulfonamide;
(Compound 425) 5-(3-Amino-phenyl)-N*3*-(4-methoxy-phenyl)-pyrazine-2,3-diamine;
(Compound 426) N*3*-(1H-Indol-5-yl)-5-(4-morpholin-4-yl-phenyl)-pyrazine-2,3-diamine;
(Compound 427) 5-(3-Amino-phenyl)-N*3*-(H-indol-5-yl)-pyrazine-2,3-diamine;
(Compound 428) 4-[5-Amino-6-(1H-indol-5-ylamino)-pyrazin-2-yl]-phenol;
(Compound 429) 5-(3-Dimethylamino-phenyl)-N*3*-(1H-indol-5-yl)-pyrazine-2,3-diamine;
(Compound 430) N*3*-(1H-Indol-5-yl)-5-(4-methanesulfonyl-phenyl)-pyrazine-2,3-diamine;
(Compound 431) N*3*-(1H-indol-5-yl)-5-(5-isopropyl-2-methoxy-phenyl)-pyrazine-2,3-diamine;
(Compound 432) N*3*-(1H-Indol-5-yl)-5-(3-methoxy-phenyl)-pyrazine-2,3-diamine;
(Compound 433) N*3*-(1H-Indol-5-yl)-5-quinolin-5-yl-pyrazine-2,3-diamine;
(Compound 434) 3-[5-Amino-6-(4-methoxy-phenylamino)-pyrazin-2-yl]-benzoic acid;
(Compound 435) 5-(4-Methanesulfonyl-phenyl)-N*3*-(4-methoxy-phenyl)-pyrazine-2,3-diamine;
(Compound 436) (E)-3-{3-[5-Amino-6-(4-methoxy-phenylamino)-pyrazin-2-yl]-phenyl}-acrylic acid;
(Compound 437) 5-(3-Bromo-phenyl)-N*3*-(4-methoxy-phenyl)-pyrazine-2,3-diamine;
(Compound 438) 5-(5-Isopropyl-2-methoxy-phenyl)-N*3*-(4-methoxy-2-methyl-phenyl)-pyrazine-2,3-diamine;
(Compound 439) {3-[5-Amino-6-(4-methoxy-2-methyl-phenylamino)-pyrazin-2-yl]-phenyl}-methanol;
(Compound 440) 3-[5-Amino-6-(4-methoxy-2-methyl-phenylamino)-pyrazin-2-yl]-benzamide;
(Compound 441) [6-(3-Amino-phenyl)-pyrazin-2-yl]-(3,4,5-trimethoxy-phenyl)-amine.

28. A compound according to any one of claims 1 to 27 for use as a pharmaceutically active agent.

29. Use of a compound according to any one of claims 1-28 as an inhibitor for a protein kinase.

30. Use of at least one compound according to one of claims 1-28 for the preparation of a pharmaceutical composition for the prophylaxis and/or treatment of infectious diseases, including opportunistic diseases, prion diseases, immunological diseases, autoimmune diseases, bipolar and clinical disorders, cardiovascular diseases, cell proliferative diseases, diabetes, inflammation, transplant rejections, erectile dysfunction, neurodegenerative diseases and stroke.

31. Use according to claim 30, wherein the virally induced infectious diseases, including opportunistic diseases, are caused by retroviruses, human endogenous retroviruses, lentiviruses, oncoretroviruses, hepadnaviruses, herpesviruses, flaviviridae, and/or adenoviruses.

32. Use according to claim 30 or 31, wherein the infective disease including opportunistic infection is selected from the group comprising AIDS, Alveolar Hydatid Disease (AHD, Echinococcosis), Amebiasis (Entamoeba histolytica Infection), Angiostrongylus Infection, Anisakiasis, Anthrax, Babesiosis (Babesia Infection), Balantidium Infection (Balantidiasis), Baylisascaris Infection (Raccoon Roundworm), Bilharzia (Schistosomiasis), Blastocystis hominis Infection (Blastomycosis), Boreliosis, Botulism, Brainerd Diarrhea, Brucellosis, BSE (Bovine Spongiform Encephalopathy), Candidiasis, Capillariasis (Capillaria Infection), CFS (Chronic Fatigue Syndrome), Chagas Disease (American Trypanosomiasis), Chickenpox (Varicella-Zoster virus), Chlamydia pneumoniae Infection, Cholera, Chronic Fatigue Syndrome, CJD (Creutzfeldt-Jakob Disease), Clonorchiasis (Clonorchis Infection), CLM (Cutaneous Larva Migrans, Hookworm Infection), Coccidioidomycosis, Conjunctivitis, Coxsackievirus A16 (Hand, Foot and Mouth Disease), Cryptococcosis, Cryptosporidium Infection (Cryptosporidiosis), Culex mosquito (Vector of West Nile Virus), Cutaneous Larva Migrans (CLM), Cyclosporiasis (Cyclospora Infection), Cysticercosis (Neurocysticercosis), Cytomegalovirus Infection, Dengue/Dengue Fever, Ebola Virus Hemorrhagic Fever, Echinococcosis (Alveolar Hydatid Disease), Encephalitis, Entomoeba coli Infection, Entomoeba dispar Infection, Entomoeba hartmanni Infection, Entomoeba histolytica Infection (Amebiasis), Entomoeba polecki Infection, Enterobiasis (Pinworm Infection), Enterovirus Infection (Non-Polio), Epstein-Barr Virus Infection, Escherichia coli Infection, Foodborne Infection, Foot and mouth Disease, Fungal Dermatitis, Gastroenteritis, Group A streptococcal Disease, Group B streptococcal Disease, Hansen's Disease (Leprosy), Hantavirus Pulmonary Syndrome, Helicobacter pylori Infection, Hematologic Disease, Hendra Virus Infection, Hepatitis (HCV, HBV), Herpes Zoster (Shingles), HIV Infection, Human Ehrlichiosis, Human Parainfluenza Virus Infection, Influenza, Isosporiasis (Isospora Infection), Lassa Fever, Leishmaniasis, Kala-azar (Kala-azar, Leishmania Infection), Leprosy, Lice (Body lice, Head lice, Pubic lice), Lyme Disease, Malaria, Marburg Hemorrhagic Fever, Measles, Meningitis, Mosquito-borne Diseases, Mycobacterium avium Complex (MAC) Infection, Naegleria Infection, Nosocomial Infections, Nonpathogenic Intestinal Amebae Infection, Onchocerciasis (River Blindness), Opisthorciasis (Opisthorcis Infection), Parvovirus Infection, Plague, PCP (Pneumocystis carinii Pneumonia), Polio, Q Fever, Rabies, Respiratory Syncytial Virus (RSV) Infection, Rheumatic Fever, Rift Valley Fever, River Blindness (Onchocerciasis), Rotavirus Infection, Roundworms Infection, Salmonellosis, Salmonella Enteritidis, Scabies, Shigellosis, Shingles, Sleeping Sickness, Smallpox, Streptococcal Infection, Tapeworm Infection (Taenia Infection), Tetanus, Toxic Shock Syndrome, Tuberculosis, Ulcers (Peptic Ulcer Disease), Valley Fever, Vibrio parahaemolyticus Infection, Vibrio vulnificus Infection, Viral Hemorrhagic Fever, Warts, Waterborne infectious Diseases, West Nile Virus Infection (West Nile Encephalitis), Whooping Cough, Yellow Fever, tuberculosis, leprosy, mycobacteria-induced meningitis.

33. Use according to claim 30, wherein the prion diseases is selected from the group comprising Scrapie, TME, CWD, BSE, CJD, vCJD, GSS, FFI, Kuru, and Alpers Syndrome.

34. Use according to claim 30, wherein the immunological disease and/or autoimmune disease is selected from the group comprising:
asthma, diabetes, rheumatic diseases, AIDS, rejection of transplanted organs and tissues, rhinitis, chronic obstructive pulmonary diseases, osteoporisis, ulcerative colitis, sinusitis, lupus erythematosus, recurrent infections, atopic dermatitis/eczema and occupational allergies, food allergies, drug allergies, severe anaphylactic reactions, anaphylaxis, manifestations of allergic diseases, primary immunodeficiencies, antibody deficiency states, cell mediated immunodeficiencies, severe combined immunodeficiency, DiGeorge syndrome, Hyper-IgE syndrome, Wiskott-Aldrich syndrome, ataxia-telangiectasia, immune mediated cancers, white cell defects, autoimmune diseases, systemic lupus erythematosus, rheumatoid arthritis (RA), multiple sclerosis (MS), immune-mediated or Type 1 Diabetes Mellitus, immune mediated glomerulonephritis, scleroderma, pernicious anemia, alopecia, pemphigus, pemphigus vulgaris, myasthenia gravis, inflammatory bowel diseases, Crohn's disease, psoriasis, autoimmune thyroid diseases, Hashimoto's disease, dermatomyositis, goodpastture syndrome, myasthenia gravis pseudoparalytica, ophtalmia sympatica, phakogene uveitis, chronical aggressive hepatitis, primary billiary cirrhosis, autoimunehemolytic anemy, Werlof disease.

35. Use according to claim 30, wherein the bipolar and/or clinical disorder is selected from the group comprising: adjustment disorders, anxiety disorders, delirium, dementia, amnestic and other cognitive disorders, disorders usually first diagnosed in infancy, childhood, or adolescence, dissociative disorders, eating disorders, factitious disorders, impulse-control disorders, mental disorders due to a general medical condition, mood disorders, other conditions that may be a focus of clinical attention, personality disorders, schizophrenia and other psychotic disorders, sleep disorders, somatoform disorders, substance-related disorders, generalized anxiety disorder, panic disorder, phobia, agoraphobia, obsessive-compulsive disorder, stress, acute stress disorder, anxiety neurosis, nervousness, phobia, posttraumatic stress disorder, posttraumatic stress disorder (PTSD), abuse, ADHD, obsessive-compulsive disorder (OCD), manic depressive psychosis, specific phobias, social phobia, adjustment disorder with anxious features.

36. Use according to claim 35, wherein the anxiety disorders, delirium, dementia, amnestic and other cognitive disorders, disorders usually first diagnosed in infancy, childhood, or adolescence, dissociative disorders, eating disorders, mood disorders, schizophrenia and other psychotic disorders, sexual and gender identity disorders, sleep disorders, somatoform disorders, substance-related disorders are selected from the group comprising: acute stress disorder, agoraphobia without history of panic disorder, anxiety disorder due to general medical condition, generalized anxiety disorder, obsessive-compulsive disorder, panic disorder with agoraphobia, panic disorder without agoraphobia, posttraumatic stress disorder, specific phobia, social phobia, substance-induced anxiety disorder, delirium due to a general medical condition, substance intoxication delirium, substance withdrawal delirium, delirium due to multiple etiologies, Alzheimer's, Creutzfeldt-Jakob disease, head trauma, Huntington's disease, HIV disease, Parkinson's disease, Pick's disease, substance-induced persisting, vascular, dementia due to other general medical conditions, dementia due to multiple etiologies, amnestic disorder due to a general medical condition, substance-induced persisting amnestic disorder, mental retardation, learning disorders, mathematics disorder, reading disorder, disorder of written expression, learning disorder, motor skills disorders, developmental coordination disorder, communication disorders, phonological disorder, pervasive developmental disorders, Asperger's disorder, autistic disorder, childhood disintegrative disorder, Rett's disorder, pervasive developmental disorder, attention-deficit/hyperactivity disorder (ADHD), conduct disorder, oppositional defiant disorder, feeding disorder of infancy or early childhood, pica, rumination disorder, tic disorders, chronic motor or vocal tic disorder, Tourette's disorder, elimination disorders, encopresis, enuresis, selective mutism, separation anxiety disorder, reactive attachment disorder of infancy or early childhood, stereotypic movement disorder, dissociative amnesia, depersonalization disorder, dissociative fugue, dissociative identity disorder, anorexia nervosa, bulimia nervosa, mood episodes, major depressive episode, hypomanic episode, manic episode, mixed episode, depressive disorders, dysthymic disorder, major depressive disorder, single episode, recurrent, bipolar disorders, bipolar I disorder, bipolar II disorder, cyclothymic disorder, mood disorder due to a general medical condition, substance-induced mood disorder, schizophreniform disorder, schizoaffective disorder, delusional disorder, brief psychotic disorder, shared psychotic disorder, psychotic disorder due to a general medical condition, delusions, hallucinations, substance-induced psychotic disorder, female sexual arousal disorder, orgasmic disorders, premature ejaculation, sexual pain disorders, dyspareunia, vaginismus, sexual dysfunction due to a general medical condition, female dyspareunia, female hypoactive sexual desire disorder, male erectile disorder, male hypoactive sexual desire disorder, male dyspareunia, other female sexual dysfunction, other male sexual dysfunction, substance-induced sexual dysfunction, sexual dysfunction, paraphilias, dyssomnias, breathing-related sleep disorder, circadian rhythm sleep disorder, hypersomnia, hypersomnia related to another mental disorder, insomnia, insomnia related to another mental disorder, narcolepsy, dyssomnia, parasomnias, nightmare disorder, sleep terror disorder, sleepwalking disorder, parasomnia, body dysmorphic disorder, conversion disorder, hypochondriasis, pain disorder, somatization disorder, undifferentiated somatoform disorder, alcohol related disorders, amphetamine related disorders, caffeine related disorders, cannabis related disorders, cocaine related disorders, hallucinogen related disorders, inhalant related disorders, nicotine related disorders, opioid related disorders, psychotic disorder, psychotic disorder, phencyclidine-related disorder, abuse, persisting amnestic disorder, anxiety disorder, persisting dementia, dependence, intoxication, intoxication delirium, mood disorder, psychotic disorder, withdrawal, withdrawal delirium, sexual dysfunction, sleep disorder.

37. Use according to claim 30, wherein the cardiovascular diseases are selected from the group consisting of:
adult congenital heart disease, aneurysm, stable angina, unstable angina, angina pectoris, angioneurotic edema, aortic valve stenosis, aortic aneurysm, arrhythmia, arrhythmogenic right ventricular dysplasia, arteriosclerosis, arteriovenous malformations, atrial fibrillation, Behcet syndrome, bradycardia, cardiac tamponade, cardiomegaly, congestive cardiomyopathy, hypertrophic cardiomyopathy, restrictive cardiomyopathy, cardiovascular disease prevention, carotid stenosis, cerebral hemorrhage, Churg-Strauss syndrome, diabetes, Ebstein's Anomaly, Eisenmenger complex, cholesterol embolism, bacterial endocarditis, fibromuscular dysplasia, congenital heart defects, heart diseases, congestive heart failure, heart valve diseases, heart attack, epidural hematoma, hematoma, subdural, Hippel-Lindau disease, hyperemia, hypertension, pulmonary hypertension, hypertrophic growth, left ventricular hypertrophy, right ventricular hypertrophy, hypoplastic left heart syndrome, hypotension, intermittent claudication, ischemic heart disease, Klippel-Trenaunay-Weber syndrome, lateral medullary syndrome, long QT syndrome mitral valve prolapse, moyamoya disease, mucocutaneous lymph node syndrome, myocardial infarction, myocardial ischemia, myocarditis, pericarditis, peripheral vascular diseases, phlebitis, polyarteritis nodosa, pulmonary atresia, Raynaud disease, restenosis, Sneddon syndrome, stenosis, superior vena cava syndrome, syndrome X, tachycardia, Takayasu's arteritis, hereditary hemorrhagic telangiectasia, telangiectasis, temporal arteritis, tetralogy of fallot, thromboangiitis obliterans, thrombosis, thromboembolism, tricuspid atresia, varicose veins, vascular diseases, vasculitis, vasospasm, ventricular fibrillation, Williams syndrome, peripheral vascular disease, varicose veins and leg ulcers, deep vein thrombosis, Wolff-Parkinson-White syndrome.

38. Use according to claim 30, wherein the proliferative disease is selected from the group comprising:
adenocarcinoma, choroidal melanoma, acute leukemia, acoustic neurinoma, ampullary carcinoma, anal carcinoma, astrocytoma, basal cell carcinoma, pancreatic cancer, desmoid tumor, bladder cancer, bronchial carcinoma, breast cancer, Burkitt's lymphoma, corpus cancer, CUP-syndrome (carcinoma of unknown primary), colorectal cancer, small intestine cancer, small intestinal tumors, ovarian cancer, endometrial carcinoma, ependymoma, epithelial cancer types, Ewing's tumors, gastrointestinal tumors, gastric cancer, gallbladder cancer, gall bladder carcinomas, uterine cancer, cervical cancer, cervix, glioblastomas, gynecologic tumors, ear, nose and throat tumors, hematologic neoplasias, hairy cell leukemia, urethral cancer, skin cancer, skin testis cancer, brain tumors (gliomas), brain metastases, testicle cancer, hypophysis tumor, carcinoids, Kaposi's sarcoma, laryngeal cancer, germ cell tumor, bone cancer, colorectal carcinoma, head and neck tumors (tumors of the ear, nose and throat area), colon carcinoma, craniopharyngiomas, oral cancer (cancer in the mouth area and on lips), cancer of the central nervous system, liver cancer, liver metastases, leukemia, eyelid tumor, lung cancer, lymph node cancer (Hodgkin's/Non-Hodgkin's), lymphomas, stomach cancer, malignant melanoma, malignant neoplasia, malignant tumors gastrointestinal tract, breast carcinoma, rectal cancer, medulloblastomas, melanoma, meningiomas, Hodgkin's disease, mycosis fungoides, nasal cancer, neurinoma, neuroblastoma, kidney cancer, renal cell carcinomas, non-Hodgkin's lymphomas, oligodendroglioma, esophageal carcinoma, osteolytic carcinomas and osteoplastic carcinomas, osteosarcomas, ovarial carcinoma, pancreatic carcinoma, penile cancer, plasmocytoma, prostate cancer, pharyngeal cancer, rectal carcinoma, retinoblastoma, vaginal cancer, thyroid carcinoma, Schneeberger disease, esophageal cancer, spinalioms, T-cell lymphoma (mycosis fungoides), thymoma, tube carcinoma, eye tumors, urethral cancer, urologic tumors, urothelial carcinoma, vulva cancer, wart appearance, soft tissue tumors, soft tissue sarcoma, Wilm's tumor, cervical carcinoma and tongue cancer.

39. Use according to claim 30, wherein said diabetes is selected from Type I diabetes or Type II diabetes.

40. Use according to claim 30, wherein said inflammation is mediated by the cytokines TNF-α, IL-1β, GM-CSF, IL-6 and/or IL-8.

41. Use according to claim 30 or 40 wherein the inflammatory disease is caused, induced, initiated and/or enhanced by bacteria, viruses, prions, parasites, fungi, and/or caused by irritative, traumatic, metabolic, allergic, autoimmune, or idiopathic reasons.

42. Use according to claim 41 wherein the viruses and bacteria are selected from the group comprising human immunodeficiency virus-I, herpes viruses, herpes simplex virus, herpes zoster virus, cytomegalovirus, mycoplasma pulmonis, ureaplasma urealyticum, mycoplasma pneumoniae, chlamydia pneumoniae, C. pneumoniae, Helicobacter pylori, and propriono-bacterium.

43. Use according to any one of the claims 30, 40-42, wherein the inflammatory disease is selected from the group comprising inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, inflammatory diseases of the larynx.

44. Use according to claim 43 wherein the inflammatory diseases of the central nervous system (CNS), inflammatory rheumatic diseases, inflammatory diseases of blood vessels, inflammatory diseases of the middle ear, inflammatory bowel diseases, inflammatory diseases of the skin, inflammatory disease uveitis, inflammatory diseases of the larynx are selected from the group comprising:
abscessation, acanthameba, acanthamebiasis, acne vulgaris, actinomycosis, acute inflammatory dermatoses, acute laryngeal infections of adults, acute multifocal placoid pigmentary epitheliopathy, acute (thermal) injury, acute retinal necrosis, acute suppurative otitis media, algal disorders, allergic contact dermatitis, amyloidosis angioedema, ankylosing spondylitis, aspergillosis, atopic dermatitis, Aujeszky's disease, autoantibodies in vasculitis, babesiosis, bacterial disorders, bacterial laryngitis, bacterial meningitis, Behcet's disease, birdshot choroidopathy, blastomycosis, borna disease, brucellosis, bullous myringitis, bursitis, candidiasis, canine distemper encephalomyelitis, canine distemper encephalomyelitis in immature animals, canine ehrlichiosis, cholesteatoma, chronic (granulomatous) diseases, chronic inflammatory dermatoses, chronic relapsing encephalomyelitis, chronic suppurative otitis media, cicatricial pemphigoid, coccidiomycosis, coccidioidomycosis, common upper respiratory infection, contact ulcer and granuloma, Crohn's disease, cryptococcosis, cysticercosis, dermatomyositis, diphtheria, discoid lupus erythematosus, drug-induced vasculitis, drug or hypersensitivity reaction, encephalitozoonosis, eosinophilic meningoencephalitis, erythemal multiforme (EM minor), feline leukemia virus, feline immunodeficiency virus, feline infectious peritonitis, feline polioencephalomyelitis, feline spongiform encephalopathy, fibromyositis, Fuch's heterochromic cyclitis, gastroesophageal (laryngopharyngeal) reflux disease, giant cell arteritis, glanders, glaucomatocyclitic crisis, gonorrhea granular myringitis, granulomatous meningoencephalomyelitis, herpes simplex, histoplasmosis, idiopathic diseases, idiopathic inflammatory disorders, immune and idiopathic disorders, infections of the immunocompromised host, infectious canine hepatitis, inhalation laryngitis, interstitial nephritis, irritant contact dermatitis, juvenile rheumatoid arthritis, Kawasaki's disease, La Crosse virus encephalitis, laryngeal abscess, laryngotracheitis (croup), leishmaniasis, lens-induced uveitis, leprosy, leptospirosis, leukemia, lichen planus, lupus, lyme disease, lymphoma, meningitis, meningoencephalitis in greyhounds, miscellaneous meningitis/meningoencephalitis, microscopic polyangiitis, multifocal choroiditis, multiple sclerosis, muscle tension dysphonias, mycotic (fungal) diseases, mycotic diseases of the CNS, necrotizing encephalitis, neosporosis, old dog encephalitis, onchocerciasis, parasitic encephalomyelitis, parasitic infections, pars planitis, parvovirus encephalitis, pediatric laryngitis, pollution and inhalant allergy, polymyositis, post-vaccinal canine distemper encephalitis, post-vaccinal rabies, prion protein induced diseases, protothecosis, protozoal encephalitis-encephalomyelitis, psoriasis, psoriatic arthritis, pyogranulomatous meningoencephalomyelitis, rabies, radiation injury, radiation laryngitis, radionecrosis, relapsing polychondritis, Reiters's syndrome, retinitis pigmentosa, retinoblastoma, rheumatoid arthritis, rickettsial disorders, rocky mountain spotted fever, salmon poisoning, sarcocystosis, sarcoidosis, schistosomiasis, scleroderma, scleroma, serpiginous choroiditis, shaker dog disease, Sjogren's syndrome, spasmodic croup, spirochetal (syphilis) diseases, spongiotic dermatitis, sporotrichosis, steroid responsive meningitis-arteritis, Stevens-Johnson syndrome (SJS, EM major), supraglottitis (epiglottitis), sympathetic ophthalmia, syngamus laryngeus, syphilis, systemic lupus erythematosus, systemic vasculitis in sarcoidosis, Takayasu's arteritis, tendinitis (tendonitis), thromboangiitis obliterans (Buerger's Disease), toxic epidermal necrolysis (TEN), toxocariasis, toxoplasmosis, trauma, traumatic laryngitis, trichinosis, trypanosomiasis, tuberculosis, tularemia, ulcerative colitis, urticaria (hives), vasculitis, vasculitis and malignancy, vasculitis and rheumatoid arthritis, vasculitis in systemic lupus erythematosus, vasculitis in the idiopathic inflammatory myopathies, vasculitis of the central nervous system, vasculitis secondary to bacterial, fungal, and parasitic infection, viral disorders, viral laryngitis, vitiligo, vocal abuse, vocal-cord hemorrhage, Vogt Koyanagi Harada syndrome, Wegener's granulomatosis, and Whipple's disease.

45. Use according to claim 30, wherein the transplant rejection is selected from the group comprising heart transplant rejection, heart-lung transplant rejection, lung transplant rejection, liver transplant rejection, kidney transplant rejection, pancreas transplant rejection, spleen transplant rejection, skin transplant rejection, tissue transplant rejection, bone marrow transplant rejection, spinal marrow transplant rejection, hormone producing glands transplant rejection, gonads and gonadal gland transplant rejection, graft-versus-host-diseases and host-versus-graft-diseases.

46. Use according to claim 30, wherein the neurodegenerative diseases are selected from the group comprising:
Alzheimer disease, Parkinson disease, Huntington disease, amyotrophic lateral sclerosis, AIDS-related dementia, retinitis pigmentosa, spinal muscular atrophy and cerebrellar degeneration, fragile X-associated tremor/ataxia syndrome (FXTAS), progressive supranuclear palsy (PSP), and striatonigral degeneration (SND), which is included with olivopontocerebellear degeneration (OPCD), and Shy Drager syndrome (SDS) in a syndrome known as multiple system atrophy (MSA).

47. Use according to claim 31, wherein the herpes virus is selected from the group comprising:
Herpes simplex viruses, varicello viruses, cytomegalo viruses, muromegalo viruses, roseolo viruses, lymphcrypto viruses and rhadino viruses.

48. Use according to claim 47, wherein the herpes virus is cytomegalovirus, preferably human cytomegalovirus (HCMV).

49. Use according to any one of claims 30-32, wherein said infectious disease is herpes.

50. Use according to claim 29, wherein the protein kinase is UL 97.

51. Use according to any one of claims 28-50, wherein at least one compound according to any one of claims 1-28 is used in combination with further therapeutic compounds, preferably selected from the group consisting of ganciclovir, foscarnet, cidofovir, valganciclovir, ganciclovir implants, fomivirsen, penciclovir and valaciclovir.

52. Pharmaceutical composition comprising at least one compound according to any one of claims 1 to 28 as an active ingredient, together with at least one pharmaceutically acceptable carrier, excipient and/or diluent.

53. Pharmaceutical composition according to claim 52 further comprising an additional therapeutic compound, preferably selected from the group consisting of ganciclovir, foscarnet, cidofovir, valganciclovir, ganciclovir implants, fomivirsen, penciclovir and valaciclovir.
